(19) United States
(12) Patent Application Publication
(10) Pub. No.: US 2013/0217949 A1
(43) Pub. Date: Aug. 22, 2013

(54) COMBINATORIAL METHODS AND COMPOSITIONS FOR TREATMENT OF MELANOMA

(71) Applicant: THE PENN STATE RESEARCH FOUNDATION, (US)

(72) Inventors: Gavin P. Robertson, Hummelstown, PA (US); Lakshman Sandirasegarane, Hershey, PA (US); Mark Kester, Harrisburg, PA (US); Arati K. Sharma, Hummelstown, PA (US)

(73) Assignee: THE PENN STATE RESEARCH FOUNDATION, University Park, PA (US)

(21) Appl. No.: 13/799,856

(22) Filed: Mar. 13, 2013

Related U.S. Application Data
(63) Continuation of application No. 13/750,836, filed on Jan. 25, 2013.
Application No. 13/750,836 is a continuation of application No. 11/083,583, filed on Mar. 18, 2005, now abandoned.
(60) Provisional application No. 60/554,509, filed on Mar. 19, 2004.

(51) Int. Cl. A61K 31/713; A61K 31/44; A61K 31/4164; A61K 38/02; A61K 45/06

(52) U.S. Cl. CPC A61K 31/713; A61K 38/02; A61K 45/06; A61K 31/4164; A61K 31/44
USPC 600/1; 514/44 A; 424/450; 514/18.9; 604/501

(57) ABSTRACT

The invention relates to combining targeted therapies with selected chemotherapeutics for the treatment of melanoma. The invention provides a method for inducing apoptosis in a melanoma tumor cell by reducing Akt3 activity, a method for inducing apoptosis in a melanoma tumor cell comprising contacting a melanoma tumor cell with an agent that reduces Akt3 activity to restore normal apoptotic sensitivity to a melanoma tumor cell, allowing a lower concentration of chemotherapeutic agents resulting in decreased toxicity to a patient. Also disclosed is a method for treating a melanoma comprising administering an agent that reduces Akt3 activity and an agent that reduces V599E B-Raf activity, thereby treating a melanoma tumor.

### REFERENCE TO RELATED APPLICATIONS

This application is a continuation of U.S. patent application Ser. No. 13/750,836, filed Jan. 25, 2013, which is a continuation of U.S. patent application Ser. No. 11/083,583, filed Mar. 18, 2005, now abandoned, which claims priority of U.S. Provisional Patent Application Ser. No. 60/554,509 filed Mar. 19, 2004. The entire content of each application is incorporated herein by reference.

### GRANT REFERENCE

This invention was made with government support under Grant No. CA127892, awarded by the National Institutes of Health. The Government has certain rights in the invention.

### BACKGROUND OF THE INVENTION

Of the three major forms of skin cancer, malignant melanoma carries the highest risk of mortality from metastasis (Schalick et al., Blackwell Science, Inc., Malden, Mass. 180-348 (1998); Jemal et al., J. Nat. Cancer Inst. 93:678-683 (2001); and Jemal et al., Ca: a Cancer Journal for Clinicians 52:23-47 (2002)). The prognosis for patients in the late stages of this disease remains very poor with average survival from six to ten months. (Jemal et al., Ca: A Cancer Journal for Clinicians 52:23-47 (2002); and Soengas et al., Oncogen 22:3138-3151 (2003)). Currently, there is no effective long-term treatment for patients suffering from the advanced stages of this cancer despite many clinical trials testing the efficacy of a wide variety of therapeutics ranging from surgery to immuno-, radio- and chemotherapy (Soengas et al., Oncogen 22:3138-3151 (2003); Serrone et al., Melanoma Res 9:51-58 (1999); Grossman et al., Cancer Metastasis Rev 20:3-11 (2001); Helmbach et al., Int J Cancer 93:617-622 (2001); Ballo et al., Surgical Clinics North Am 83:323-342 (2003); and Hersey, P., Int Med J 33:33-43 (2003)). The lack of effective therapeutic regimes is due, in part, to a lack of information about the predominant genes altered during melanoma development, and therapies specifically targeted to correct these defects (Serrone et al., J Exp Clin Cancer Res 19:21-34 (2000); and Atkins et al., Nature Rev. Drug Dis 1:491-492 (2002)).

Patients with metastatic (Stage 1V) malignant melanoma have a median survival of approximately one year (Balch et al., 1993; Koh, 1991). Current standard treatment consists of combination chemotherapy with agents such as cisplatin, DTIC, and BCNU, with or without cytokines such as interleukin-2 (IL-2) or interferon-.alpha. (IFN-.alpha.) (Balch et al., 1993; Koh, 1991; Legha and Buzaid, 1993). Response rates to chemotherapy have been reported to be as high as 60%, yet only approximately 5% of patients experience long-term survival, regardless of the therapeutic regimen employed. Conventional chemotherapy aims to control the growth of cancer by targeting rapidly growing cells. However, this function is not specific, as many normal cells, such as those of the bone marrow and the intestinal epithelium, also have a basal level of proliferation. Therefore, many normal cells of the body also are susceptible to the toxic effects of chemotherapy, and conventional chemotherapy can impart a substantial degree of morbidity to the patient. Clearly, new approaches to the treatment of metastatic melanoma are needed.

The Akt protein kinase family consists of three members, Akt1/PKBα, Akt2/PKBβ and Akt3/PKBγ, which share a high degree of structural similarity (Brazil et al., Cell 111:293-303 (2002); and Nicholson et al., Cell Signal 14:381-395 (2002)). Family members share extensive structural similarity with one another, exhibiting greater than 80% homology at the amino acid level (Nicholson K M, Anderson N G. Cell Signal. 14(5):381-95 (2002), Datta S R et al. Genes Dev. 13(22):2905-27 (1999).). All Akt isoforms share major structural features, having three distinct functional domains (Testa J R, Bellacosa. A. Proc Natl Acad Sci USA. 98(20):10983-5 (2001), Nicholson K M, Anderson N G. Cell Signal. 14(5):381-95 (2002), Scheid M P, Woodgett J R. Nat Rev Mol Cell Biol. 2(10):760-8 (2001), Scheid M P, Woodgett J R. FEBS Lett. 546(1):108-12 (2003), Bellacosa A et al. Cancer Biol Ther. 3(3):268-75. Epub 2004 (2004), Brazil D P et al. Trends Biochem Sci. 29(5):233-42 (2004), Brazil, D. P. et al. Cell 111:293-303 (2002), Brazil D P, Hemmings B A. Trends Biochem Sci. 26(11):657-64 (2001), Datta S R et al. Genes Dev. 13(22):2905-27 (1999).). One is an amino-terminal pleckstrin homology domain (PH) domain that mediates protein-protein and protein-lipid interactions. This domain consists of approximately one hundred amino acids, resembles the three phosphoinsitides binding domains in other signaling molecules (Lietzke S E et al. Mol. Cell. 6(2):385-94 (2000), Ferguson K M et al. Mol. Cell. 6(2):373-84 (2000).). The second domain is a carboxy-terminal kinase catalytic region that mediates phosphorylation of substrate proteins. It shows a high degree of similarity to those in protein kinase A (PKA) and protein kinase C (PKC) (Jones P F et al. Cell Regul. 2(12):1001-9 (1991). Andjelkovic M, Jones P F, Grossniklaus U, Cron P, Schier A F, Dick M, Bilbe G, Hemmings B A. Developmental regulation of expression and activity of multiple forms of the Drosophila RAC protein kinase. J Biol. Chem. 270(8):4066-75 (1995).). The third domain is a tail region with an important regulatory role. This region is sometimes referred to as the tail or regulatory domain. Within the latter two regions are serine and threonine residues whose phosphorylation is required for Akt activation. The sites vary slightly dependent of the particular Akt isoform. The first site on all three isoforms is a threonine at amino acid position 308/309/305 and on Akt1/2/3 respectively. The second site is a serine occurring within the hydrophobic C-terminal tail at amino acid positions 473/474/472 on Akt 1/2/3 respectively. Phosphorylation on both sites occurring in response to growth factors or other extracellular stimuli is essential for maximum Akt activation (Alessi D R, Andjelkovic M, Caudwell B, Cron P, Morrice N, Cohen P, Hemmings B A. Mechanism of activation of protein kinase B by insulin and IGF-1. EMBO J. 15(23):6541-51 (1996).). Akt may also be phosphorylated on other residues; however, the functional significance of this phosphorylation is an area of continuing investigation (Alessi D R, Andjelkovic M, Caudwell B, Cron P, Morrice N, Cohen P, Hemmings B A. Mechanism of activation of protein kinase B by insulin and IGF-1. EMBO J. 15(23):6541-51 (1996).). Also, although splice variants of Akt3 lacking the serine 472 phosphorylation site have been identified, the cellular role of this variant remains uncertain (Brodbeck D, Hill M M, Hemmings B A. J Biol. Chem. 276(31):29550-8. Epub 2001 (2001).). It is also unknown whether this variant is present or performs any role in the melanoma cells.

While all isoforms may be expressed in a particular cell type, only certain isoforms may be active. It also appears that each isoform can perform unique as well as common functions in cells (Brazil et al., Cell 111:293-303 (2002); and Nicholson et al., Cell Signal 14:381-395 (2002); Chen et al., Genes Dev 15:2203-2208 (2001); and Cho et al., Science 292:1728-1731 (2001)). Knockout mice lacking Akt1 are growth retarded and have increased rates of spontaneous apoptosis in the testis and thymus (Chen et al., Genes Dev 15:2203-2208 (2001); Cho et al., J Biol Chem 276:38349-38352 (2001); Peng et al., Genes Dev 17:1352-1365 (2003)). In contrast, Akt2 knockout mice have impaired insulin regulation and consequently a defective capability of lowering blood glucose levels due to defects in the action of insulin on liver and skeletal muscle (Cho et al., Science 292:1728-1731 (2001); Peng et al., Genes Dev 17:1352-1365 (2003)). Currently, there is no published report describing the phenotype associated with an Akt3 knockout mouse; thus, there is very little known about the specific functions of Akt3 or its role in human cancer.

Genetic amplification that increase the expression of Akt1 or Akt2 have been reported in cancers of the stomach, ovary, pancreas and breast (Staal, S. P., Proc Nat Acad Sciences ISA 84:5034-5037 (1987); Cheng et al., Proc Nat Acad Sciences USA 89:9267-9271 (1992); Cheng et al., Proc Nat Acad Sciences USA 93:3636-3641 (1996); Lu et al., Chung-Hua I Hsuch Tsa Chih [Chinese Medical Journal] 75:679-682 (1995); Bellacosa et al., Int J Cancer 64:280-285 (1995); and van Dekken et al., Cancer Res 59:749-752 (1999)). While no activating mutations of Akt have been identified in melanomas (Waldmann et al., Arch Dermatol Res 293:368-372 (2001); Waldmann et al., Melanoma Res 12:45-50 (2002)), blocking total Akt function by targeting P13K (with the P13K inhibitors Wortmannin or LY-294002) inhibits cell proliferation and reduces the sensitivity of melanoma cells to UV radiation (Krasilnikov et al., Mol Carcinogenesis 24:64-69 (1999)). Total Akt activity has also been measured in melanomas using immunohistochemistry to demonstrate increased levels of total phosphorylated Akt in severely dysplastic nevi and metastatic melanomas compared to normal or mildly dysplastic nevi (Dhawan et al., Cancer Res 62:7335-7342 (2002)). However, the role played by individual Akt isoforms and mechanisms leading to deregulation of particular Akt isoforms in melanoma is unknown. Recently, the phosphoinositide 3-kinase (PI3K)/Akt signaling pathway was found to play a critical role in melanoma tumorigenesis (Stahl et al., Cancer Res 63:2891-2897 (2003)). Deregulated Akt activity through loss of the PTEN phosphatase, a negative regulator of P13K/Akt signaling, was found to decrease the apoptotic capacity of melanoma cells and thereby regulate melanoma tumorigenesis (Stahl et al., Cancer Res 63:2891-2897 (2003)).

The Raf protein serine/threonine kinase family consists of three members, A-Raf, B-Raf, and C-Raf. (Mercer et al., Biochim Biophys Acta 1653:25-40 (2003)). Raf family members are intermediate molecules in the MAPK (Ras/Raf/MAPK kinase (MEK)/extracellular signal-regulated kinase (ERK) pathway, which is a signal transduction pathway that relays extracellular signals from cell membrane to nucleus via an ordered series of consecutive phosphorylation events (Mercer et al., Biochim Biophys Acta 1653:25-40 (2003), Smalley. Int J Cancer 104: 527-32 (2003)). Typically, an extracellular ligand binds to its tyrosine kinase receptor, leading to Ras activation and initiation of a cascade of phosphorylation events (Mercer et al., Biochim Biophys Acta 1653:25-40 (2003), Smalley. Int J Cancer 104: 527-32 (2003)). Activated Ras causes phosphorylation and activation of Raf, which in turn phosphorylates and activaters MEK1 MEK2. MEK kinases in turn phosphorylate and activate ERK1 and ERK2 (Chong et al, Cell Signal 15:163-69 (2003)), which phosphorylates several cytoplasmic and nuclear targets that ultimately lead to expression of proteins playing important roles in cell growth and survival (Chang et al., Int J Oncol 22:469-80 (2003)).

Mutations that lead to activation of B-Raf have been found in the majority of sporadic melanomas, mainly B-RAF the most mutated gene in melanomas with a mutation rate ranging from 60 to 90% (Davies et al., Nature 417:949-54 (2002); Pollock et al., Nat Genet. 33:19-20 (2003); Brose et al., Cancer Res 62:6997-7000 (2002); and Yazdi et al., J Invest Dermatol 121:1160-62 (2003)). The majority of B-RAF mutations occur as a result of a single base missense substitution that converts T to A at nucleotide 1796 which substitutes a Valine for a Glutamic Acid at codon 599 (V599E) in exon 15 (Davies et al., Nature 417:949-54 (2002)). This mutation increases basal kinase activity of B-Raf, resulting in hyperactivity of the MAPK pathway evidenced by constitutively elevated levels of downstream kinases MEK and ERK (Davies et al., Nature 417:949-54 (2002)). B-RAF mutations are acquired, somatic, post-zygotic events that have not been identified in familial melanomas (Lang et al. Hum Mutat 21:327-30 (2003); Laud et al, Cancer Res 63:3061-65 (2003); and Meyer et al, Int J Cancer 106:78-80 (2003)).

RNA interference (RNAi) is a polynucleotide sequence-specific, post-transcriptional gene silencing mechanism effected by double-stranded RNA that results in degradation of a specific messenger RNA (mRNA), thereby reducing the expression of a desired target polypeptide encoded by the mRNA (see, e.g., WO 99/32619; WO 01/75164; U.S. Pat. No. 6,506,559; Fire et al., Nature 391:806-11 (1998); Sharp, Genes Dev. 13:139-41 (1999); Elbashir et al. Nature 411:494-98 (2001); Harborth et al., J. Cell Sci. 114:4557-65 (2001)). RNAi is mediated by double-stranded polynucleotides as also described herein below, for example, double-stranded RNA (dsRNA), having sequences that correspond to exonic sequences encoding portions of the polypeptides for which expression is compromised. RNAi reportedly is not effected by double-stranded RNA polynucleotides that share sequence identity with intronic or promoter sequences (Elbashir et al., 2001). RNAi pathways have been best characterized in Drosophila and Caenorhabditis elegans, but “small interfering RNA” (siRNA) polynucleotides that interfere with expression of specific polypeptides in higher eukaryotes such as mammals (including humans) have also been considered (e.g., Tuschl, 2001 Chembiochem. 2:239-245; Sharp, 2001 Genes Dev. 15:485; Bernstein et al., 2001 RNA 7:1509; Zamore, 2002 Science 296:1265; Plasterk, 2002 Science 296:1263; Zamore 2001 Nat. Struct. Biol. 8:746; Matzke et al., 2001 Science 293:1080; Scadden et al., 2001 EMBO Rep. 2:1107).

According to a current non-limiting model, the RNAi pathway is initiated by ATP-dependent, processive cleavage of long dsRNA into double-stranded fragments of about 18-27 (e.g., 19, 20, 21, 22, 23, 24, 25, 26, etc.) nucleotide base pairs in length, called small interfering RNAs (siRNAs) (see review by Hutvagner et al., Curr. Opin. Gen. Dev. 12:225-32 (2002); Elbashir et al., 2001; Nyknen et al., Cell 107:309-21 (2001); Bass, Cell 101:235-38 (2000)); Zamore et al., Cell 101:25-33 (2000)). In Drosophila, an enzyme known as “Dicer” cleaves the longer double-stranded RNA into siRNAs; Dicer belongs to the RNase III family of dsRNA-specific endonucleases (WO 01/68836; Bernstein et al., Nature 409:363-66 (2001)). Further according to this non-limiting model, the siRNA duplexes are incorporated into a protein complex, followed by ATP-dependent unwinding of the siRNA, which then generates an active RNA-induced silencing complex (RISC) (WO 01/68836). The complex recognizes and cleaves a target RNA that is complementary to the guide strand of the siRNA, thus interfering with expression of a specific protein (Hutvagner et al., supra).

In C. elegans and Drosophila, RNAi may be mediated by long double-stranded RNA polynucleotides (WO 99/32619; WO 01/75164; Fire et al., 1998; Clemens et al., Proc. Natl. Acad. Sci. USA 97:6499-6503 (2000); Kisielow et al., Biochem. J. 363:1-5 (2002); see also WO 01/92513 (RNAi-mediated silencing in yeast)). In mammalian cells, however, transfection with long dsRNA polynucleotides (i.e., greater than 30 base pairs) leads to activation of a non-specific sequence response that globally blocks the initiation of protein synthesis and causes mRNA degradation (Bass, Nature 411:428-29 (2001)). Transfection of human and other mammalian cells with double-stranded RNAs of about 18-27 nucleotide base pairs in length interferes in a sequence-specific manner with expression of particular polypeptides encoded by messenger RNAs (mRNA) containing corresponding nucleotide sequences (WO 01/75164; Elbashir et al., 2001; Elbashir et al., Genes Dev. 15:188-200 (2001)); Harborth et al., J. Cell Sci. 114:4557-65 (2001); Carthew et al., Curr. Opin. Cell Biol. 13:244-48 (2001); Mailand et al., Nature Cell Biol. Advance Online Publication (Mar. 18, 2002); Mailand et al. 2002 Nature Cell Biol. 4:317).

siRNA polynucleotides may offer certain advantages over other polynucleotides known to the art for use in sequence-specific alteration or modulation of gene expression to yield altered levels of an encoded polypeptide product. These advantages include lower effective siRNA polynucleotide concentrations, enhanced siRNA polynucleotide stability, and shorter siRNA polynucleotide oligonucleotide lengths relative to such other polynucleotides (e.g., antisense, ribozyme or triplex polynucleotides). By way of a brief background, “antisense” polynucleotides bind in a sequence-specific manner to target nucleic acids, such as mRNA or DNA, to prevent transcription of DNA or translation of the mRNA (see, e.g., U.S. Pat. No. 5,168,053; U.S. Pat. No. 5,190,931; U.S. Pat. No. 5,135,917; U.S. Pat. No. 5,087,617; see also, e.g., Clusel et al., 1993 Nucl. Acids Res. 21:3405-11, describing “dumbbell” antisense oligonucleotides). “Ribozyme” polynucleotides can be targeted to any RNA transcript and are capable of catalytically cleaving such transcripts, thus impairing translation of mRNA (see, e.g., U.S. Pat. No. 5,272,262; U.S. Pat. No. 5,144,019; and U.S. Pat. Nos. 5,168,053, 5,180,818, 5,116,742 and 5,093,246; U.S. 2002/193579). “Triplex” DNA molecules refer to single DNA strands that bind duplex DNA to form a collinear triplex molecule, thereby preventing transcription (see, e.g., U.S. Pat. No. 5,176,996, describing methods for making synthetic oligonucleotides that bind to target sites on duplex DNA). Such triple-stranded structures are unstable and form only transiently under physiological conditions. Because single-stranded polynucleotides do not readily diffuse into cells and are therefore susceptible to nuclease digestion, development of single-stranded DNA for antisense or triplex technologies often requires chemically modified nucleotides to improve stability and absorption by cells. siRNAs, by contrast, are readily taken up by intact cells, are effective at interfering with the expression of specific polypeptides at concentrations that are several orders of magnitude lower than those required for either antisense or ribozyme polynucleotides, and do not require the use of chemically modified nucleotides.

Malignant melanoma is the skin cancer with the most significant impact on man carrying the highest risk of death from metastasis. Both incidence and mortality rates continue to rise each year, with no effective long-term treatment on the horizon. In part, this reflects lack of identification of critical genes involved and specific therapies targeted to correct these defects. Accordingly, a need exist in the art for identification of critical genes involved and specific therapies targeted to correct these defects, and targeted reduction of gene(s) identified as key in the PI3K/Akt and MEK/ERK signaling pathways. Identifying a gene as a selective target provides new therapeutic opportunities for melanoma patients.

Therefore, it is a primary object, feature, or advantage of the present invention to improve upon the state of the art.

It is a further object, feature, or advantage of the present invention to provide a method for reducing Akt3 activity in a cancer cell, thereby restoring normal apoptotic sensitivity to a cancer cell.

It is a further object, feature, or advantage of the present invention to provide a method for inducing apoptosis in a cancer cell with an agent that reduces Akt3 activity.

It is a further object, feature, or advantage of the present invention to provide a combinatorial approach of treating melanomas by restoring normal apoptotic sensitivity to a melanoma tumor cell, decreasing cell proliferation and growth of the melanoma tumor cell, and inhibiting vascularization of the melanoma tumor cell.

It is a further object, feature, or advantage of the present invention to provide a method of treating melanomas that reduces tumor size more efficiently than conventional methods.

It is a further object, feature, or advantage of the present invention to provide a method of treating melanomas that requires a lower concentration of chemotherapy to be used, thereby decreasing toxicity to the patient.

These and other objects, features, or advantages will become apparent from the following description of the invention.

### SUMMARY OF THE INVENTION

The present invention provides a rational basis for combining targeted therapies together with selected chemotherapeutics, which does not currently exist for the treatment of melanoma. The present invention is based on the present inventors' discovery that Akt3 regulates apoptosis and V599E B-Raf regulates growth and vascular development in melanoma. Inventors are the first to recognize an effective combined targeted therapeutic for treating melanoma. In one embodiment, the invention provides a method for inducing apoptosis in a melanoma tumor cell by reducing Akt3 activity. In yet another embodiment, the invention provides a method for inducing apoptosis in a melanoma tumor cell comprising contacting a melanoma tumor cell with an agent that reduces Akt3 activity. Consequently, the method provided restores normal apoptotic sensitivity to a melanoma tumor cell, thereby allowing the administration of a lower concentration of chemotherapeutic agents resulting in decreased toxicity to a patient.

The present inventors' contemplate a method for treating a melanoma tumor in a mammal comprising: administering to a melanoma tumor an effective amount of an agent to induce apoptosis; and administering to a melanoma tumor an effective amount of an agent to reduce angiogenesis and cell proliferation.

Also disclosed herein is a method for treating a melanoma in a mammal comprising: administering to a melanoma tumor in a mammal an effective amount of an agent that reduces Akt3 activity; administering to a melanoma tumor in a mammal an effective amount of an agent that reduces V599E B-Raf activity, thereby treating a melanoma tumor.

In another aspect, the invention provides a pharmaceutical composition for treating a melanoma tumor comprising: an agent that reduces Akt3 activity; and a carrier.

These and other embodiments of the invention will become apparent upon reference to the following Detailed Description. All references disclosed herein are hereby incorporated by reference in their entireties as if each was incorporated individually.

### BRIEF DESCRIPTION OF THE DRAWINGS

FIG. 1 shows identification of Akt3 involvement in malignant melanoma. A. Akt activity in the melanoma cell line UACC 903 is regulated by PTEN. Western blot analysis showing expression of phosphorylated-Akt, total Akt, PTEN and α-enolase (loading control). The 36A, 29A and 37A cell lines are genetically related cell lines created from the UACC 903 parental cell line that expresses PTEN. Tumorigenic revertant cell lines derived from the 36A cell line are considered isogenic, differing only in PTEN expression. Melanocytes serve as a control for normal cells. The graph represents densitometric scans from 3 separate Western blots to quantitatively demonstrate the level of phosphorylated to total Akt in each cell line; bars, ±SEM; statistics, One-Way ANOVA followed by Dunnet's Multiple Comparisons versus the melanocyte control, *P<0.5. B. SiRNA for each of the Akt isoforms demonstrates specificity of knockdown of each ectopically expressed Akt isoform in the UACC 903 cell line. Constructs expressing tagged HA-Akt1, HA-Akt2 or HA-Akt3 were co-nucleofected together with siRNA specific to Akt1, Akt2 or Akt3 into UACC 903 cells. Controls were non-nucleofected or vector only nucleofected cells. Western blots were probed with antibodies to HA to detect the ectopically expressed protein as well as for α-enolase, which served as a loading control. C. SiRNA mediated knockdown of Akt3, but not Akt2 or Akt2, alters level of phosphorylated Akt (activity) in the melanoma cell lines UACC 903, WM1 15 and SK-MEL-24. Western blot analysis showing expression of phosphorylated-Akt, Akt3, Akt2 and α-enolase following nucleofection with 50 (left) or 100 pmoles (right) for each respective siRNA. Controls were non-nucleofected or cells nucleofected with scrambled siRNA. Data are representative of a minimum 2 separate experiments. The loading control for these experiments was α-enolase. D. Phosphorylated Akt3 is reduced when PTEN protein is present in the UACC 903 (PTEN) tumorigenic model. Akt3 and Akt2 were immunoprecipitated from cell lines in the UACC 903 (PTEN) tumorigenic model and analyzed by Western blotting with an antibody recognizing phosphorylated Akt. The PTEN expressing 36A, 29A and 37A cell lines were derived from the UACC 903 parental cell line that lacks PTEN protein. The two tumorigenic revertant cell lines were derived from the 36A cell line and no longer express PTEN. A negative antigen control is shown together with positive and negative controls for Akt3 and Akt2. Controls for Akt3 and Akt2 were HEK 298T or LNCaP cells respectively, untreated (positive) or treated with LY-294002 (negative), an inhibitor of P 13K. E. Akt3 activity is reduced in the presence of PTEN in the UACC 903 (PTEN) tumorigenic model. Immunoprecipitated Akt3 was used in an in vitro kinase assay in which Crosstide was phosphorylated by Akt3 to estimate activity. Plot shows activity after subtraction of the no antigen control; bars, ±SEM; statistics, One-Way ANOVA followed by Dunnet's Multiple Comparisons versus the melanocyte control, *P<0.05.

FIG. 2 shows increased Akt3 expression and activity occur during melanoma tumor progression. A. An increase in the level of phosphorylated (active) Akt occurs during the radial growth phase in the melanoma tumor progression model. Western blot comparing amount of phosphorylated Akt in melanocytes to low passage melanoma cell lines established from primary tumors at the radial (WM35 and WM3211) and vertical (WM115, WM98.1 and WM278) stages of growth. Total Akt is shown as a control. B. Comparison of Akt3 versus Akt2 expression in the melanoma tumor progression model. Western blots showing the levels of expression of Akt3 and Akt2 are shown together with α-enolase as a loading control. C. Akt3 is preferentially activated in cell lines of the melanoma tumor progression model compared to Akt2. Akt3 or Akt2 was immunoprecipitated from each cell line and subject to Western blot analysis to measure the amount of phosphorylated Akt in the immunoprecipitate. D. Akt3 is preferentially overexpressed in metastatic melanomas from human patients compared to melanocytes. Akt3 and Akt2 expression were measured from metastatic melanomas derived from 31 tumors. Akt3 and Akt2 expression was normalized to α-enolase expression. The graph quantitatively compares the level of Akt3 or Akt2 expression in each tumor versus melanocytes. Bars represent average values from densitometric scans of 3 separate Western blots; bars, ±SEM. Value above represents the fold increase in expression over that occurring in melanocytes; only differences of ≧2-fold were scored as significant. E. Expression and activity of Akt3, but not Akt2, increases in tumors from melanoma patients compared to melanocytes. Activity was determined by immunoprecipitation of Akt3 and Akt2 followed by Western blot analysis with an antibody recognizing phosphorylated Akt to determine the percentage of tumors in which phosphorylated (active) Akt3 or Akt2 could be detected; statistics, t-test, *P<0.05.

FIG. 3 shows the mechanism underlying deregulated Akt3 activity in malignant melanomas. A. Decreased PTEN expression (activity) specifically increases Akt3 activity in melanocytes and: B. radial growth phase WM35 (radial growth phase) cells. SiRNA mediated reduction of PTEN is shown alone (control) or in combination with scrambled siRNA or with siRNA against Akt1, Akt2 or Akt3. Western blot analysis shows expression of phosphorylated Akt, Akt3, Akt2 and PTEN. α-enolase served as a loading control. C. Over expression of Akt3 in human melanocytes increases the levels of phosphorylated Akt. Wild type Akt3, dead Akt3 (inactive) or myristoylated Akt3 (active) were nucleofected into melanocytes. Similar constructs for Akt2 served as controls (data not shown). Akt phosphorylation (activity) was measured by Western blot analysis to measure levels of phosphorylated Akt. Arrowhead shows location endogenously active Akt3 while arrow indicates ectopically expressed active HA-tagged Akt3.

FIG. 4 shows increased Akt3 activity promotes melanoma tumor development by reducing apoptosis rates. A. PTEN-mediated reduction of Akt3 activity inhibits melanoma tumor development. Size of tumors formed by parental UACC 903 melanoma cells, the isogenic 36A (retaining PTEN) and revertant cell line (lacking PTEN) were measured 10 days after injection into nude mice. Values are means of a minimum of six injection sites in three mice per cell line, bars, ±SEM; statistics, One-Way ANOVA followed by Dunnet's Multiple Comparisons versus UACC 903, *P<0.05. B. SiRNA mediated down-regulation of Akt3 reduces the tumorigenic potential of UACC 903 melanoma cells. SiRNA against Akt3, Akt2 and Akt1 were nucleofected into UACC 903 cells and after 48 hours, cells were injected into nude mice. Size of tumors was measured 10 days later. Controls are UACC 903 cells nucleofected with buffer only or a scrambled siRNA. Values are means of a minimum of six injection sites in three mice per cell line; bars, ±SEM; statistics, One-Way ANOVA followed by Dunnet's Multiple Comparisons versus UACC 903, *P<0.05. C.D.E.F. PTEN or siRNA-mediated reduction of Akt3 increases apoptosis in tumors growing in nude mice. Quantification (C, D) and photographs (E, F) of TUNEL positive cells in tumor masses derived from UACC 903 cells expressing PTEN (36A) or nucleofected with siRNA to siAkt3 and siAkt2; bars, ±SEM; statistics, Kruskal-Wallis followed by Dunnet's Multiple Comparisons versus UACC 903, *P<0.05. Tumors were analyzed 4 days after injection of cells into nude mice; magnification, 200×. The controls were UACC 903 cells or UACC 903 cells nucleofected with buffer only. White nuclei represent cells undergoing apoptosis.

FIG. 5 depicts a demonstration that liposomes alone are non-toxic to melanoma cells. Addition of liposomes at various concentrations did not reduce the number of viable cells. In fact, they increased cell viability at all concentrations by 48 hours. Methods: Toxicity of liposomes was evaluated in 1205 Lu using the MIS assays as 24, 48, and 72 hours after addition of liposomes at concentrations of 6.25, 12.5, 25, and 50 uM.

FIG. 6 depicts a demonstration that melanoma cells readily take up liposomes. Methods: Labeled liposomes (green) were added to AUCC 903 melanoma cells growing in culture. Images show cell nuclei on left (counterstained with DAPI) and cells that have taken up labeled liposomes on the right; magnification, 40×. Approximately, 99% of cells take-up liposomes.

FIG. 7 depicts a quantitation of liposome uptake by melanoma cells. Methods: Labeled liposomes or liposomes containing labeled siRNA were added to cells growing in culture at a concentration of 20 nM. One hour later cells were fixed with 5% paraformaldehyde, counterstained with DAPI and % of cells that had taken-up labeled product were scored.

FIG. 8 depicts a demonstration of size uniformity and size distribution of liposomes. Methods: Left: Scanning Electron Micrograph showing uniform size of liposomes. Right: Size distribution of liposomes determined by light scattering analysis. Graph shows size range of liposomes, with the average size occurring between 70-80 nm.

FIG. 9 depicts a demonstration of liposomes delivering pools of siRNA to melanoma cells. Methods: Red and green labeled siRNA were added to melanoma cells growing in culture. One hour after uptake the cells were fixed in 4% paraformaldehyde and counterstained with DAPI. The left shows the cell nuclei stained blue, followed by red siRNA and green siRNA. The last column is the merged image, magnification 40×.

FIG. 10 depicts a demonstration of duration of Stealth siRNA knockdown of protein expression in 1205 Lu melanoma cells. Methods: The duration of protein knockdown by Stealth siRNA from Invitrogen was determined to be beyond 8 days. SiRNA was transferred into the 1205 Lu melanoma cell line by nucleofection. Western blot analysis of B-Raf protein levels was measured at 2-day intervals up to day 8. SiRNA against C-Raf served and a control. In addition to decreased B-Raf expression, activity of the pErk 1′/2 downstream in the signaling pathway was also decreased for 8 days. Erk-2 served as a protein loading control.

FIG. 11 depicts a demonstration of knockdown of protein expression following liposome mediated delivery of siRNA into melanoma cells. SiRNA liposome complexes targeted to mutant B-Raf can knockdown 50% of protein expression at 200 nM. This indicates a base line; higher concentrations will increase knockdown. Methods: siRNA liposome complexes were added to cells at a concentration of 100 or 200 nM. Lysates were collected after 72 hours and analyzed by Western blot.

FIG. 12 shows siRNA-mediated reduction of mutant ^{V599E}B-Raf reduces the downstream activity of MEK and ERK in melanoma. SiRNA-mediated knockdown of B-Raf and C-Raf reduces levels of each respective protein 24 and 48 hours after nucleofection in melanoma cell lines UACC 903 (A), 1205 Lu (B), and C8161 (C). Scrambled siRNA was used as a control, whereas lamin A/C siRNA was used as an additional control for UACC 903 cells. Only siRNA to B-Raf reduced the levels of active (phosphorylated) MEK and ERK downstream of B-Raf in UACC 903 and 1205 LU cells containing mutant ^{V599E}B-Raf. ERK2 is used as a loading control.

FIG. 13 shows melanoma tumor development was inhibited with ^{V599E}B-Raf but not siRNA to C-RAF or scrambled siRNA. siRNA-mediated knockdown of B-Raf protein persists for 6 to 8 days after nucleofection into UACC 903 (A) and 1205 Lu (B) cell lines growing in culture. A corresponding decrease was observed in phosphorylated ERK1/ERK2 levels (B). ERK2 served as a loading control. siRNA-mediated reduction of B-Raf led to decreased tumorigenic potential of UACC 903 (C) and 1206 Lu (D) cells. siRNA against B-Raf, C-Raf and scrambled siRNA were introduced into UAC 903 or 1205 Lu cells (white arrow) and 36 hours later cells were injected into nude mice (black arrow). Size of tumors was measured at 2-day intervals. siRNA-mediated down-regulation of B-Raf reduced the tumorigenic potential of UACC 903 and 1205 Lu melanoma cells. Controls cells were nucleofected with buffer only, a scrambled siRNA or siRNA against C-Raf. Values are means of minimum of 12 injection sites in six mice with two separate experiments. Bars ±SE.

FIG. 14 shows pharmacologic inhibition of B-Raf activity using BAY 43-9005 inhibits melanoma tumor development. A, BAY 43-9006 inhibits both wild-type and mutant ^{V599E}B-Raf activity. HA-tagged wild-type or mutant ^{V599E}B-Raf were expressed in HEK 293T cells exposed to 5 μmol/L BAY 43-9006 or DMSO vehicle. HA indicates ectopically expressed B-Raf protein. Activation or inhibition of the MAPK pathway was determined by comprising levels of pMEK and pERK, ERK2 served as a loading control.; B, BAY 43-9006 decreases pMEK and PERK (activity) levels in UACC 903 melanoma cells containing mutant ^{V599E} B-Raf in a dose responsive manner. Western blot analysis of reduced pMEK and PERK levels in UACC 903 cells with increasing concentrations of BAY 43-9006. The loading control was ERK2. C, pretreatment of mice with BAY 43-9006 inhibits development of melanoma tumors. Four days before injection of 5×10⁸ UACC 903 cells, mice were pretreated twice i.p. with 50 mg/kg BAY 43-9006 or DMSO vehicle, which continued every 2 days (arrowheads). Tumor size is shown at 2-day intervals up to day 22. Bars, ±SE D, decreased tumor cell proliferation accompanies siRNA-mediated inhibition of melanoma tumor development. Five- to 8-fold decrease in bromodeoxyuridine-positive cells occurs following siRNA-mediated inhibition of B-Raf but no C-Raf or scrambled siRNA. P<0.05. Columns, means from six different tumors with four to six fields counted per tumor; bars, ±SE.

FIG. 15 shows inhibition of B-Raf activity using BAY 43-9006 inhibits melanoma tumor development. The effects of BAY 43-9006 treatment are shown on UACC 903(A) and 1205 Lu (B) tumor development. UACC 903 and 1205 Lu cells were injected into nude mice and tumor development allowed to occur to day 6 at which point mice were injected i.p. every 2 days with BAY 43-9006 dissolved in DMSO (arrowheads). Control conditions were DMSO treatment only. The Raf kinase inhibitor BAY 43-9006 reduces the tumorigenic potential of melanoma cells containing mutant ^{V599E}B-Raf protein at concentrations ≧50 mg/kg. C, decreased amounts of phosphorylated (active) ERK were observed for those cells following treatment with BAY 43-9006 but not with vehicle treatment. Immunohistochemical comparison of the number of pERK-positive cells in UACC 903 tumor sections treated with 50 mg/kg BAY 43-9006 (in DMSO) or in DMSO vehicle alone. A 3-fold difference was detected between control vehicle and BAY 43-9006 treated cells (D). *. P<0.05. Columns, means from six different tumors with four to six fields counted per tumor; bars, ±SE.

FIG. 16 shows mechanism underlying inhibition of melanoma tumor development following pharmacologic or siRNA-mediated inhibition of mutant ^{V599E}B-Raf in melanoma tumors. Comparison of the vascular development (A), apoptosis (B), and proliferation rates (C) in temporally and spatially matched tumors exposed to BAY 43-9006 or vehicle (DMSO). Size- and time-matched tumors developing in parallel were compared to identify the effects of B-Raf inhibition on tumor development. A difference in vascular development was the first statistically significant different (*, P<0.05) observed following treatment of UACC 903 tumors with BAY 43-9006, which was followed by increased apoptosis (*, P<0.05) and reduced cell proliferation (*; P<0.05). Columns, means from two separate experiments with four to six fields analyzed from each of six tumors per experiment; bars, ±SE.

FIG. 17 shows siRNA and pharmacologic inhibition of ^{V599E}B-Raf reduces VEGF secretion from melanoma cells. VEGF secretion was measured from UACC 903 or 1205 Lu cells growing in culture by ELISA assay following nucleofection with either B-Raf or VEGF siRNA (A) or after treatment with increasing concentrations of BAY 43-9006 (B). C-Raf and scrambled siRNA served as controls. Bars, ±SD. The effects of reduced VEGF expression are shown on UACC 903 (C) and 1205 Lu (D) tumor development. Tumor size is shown at 2-day intervals up to day 17.5. Reduction of VEGF expression inhibits melanoma tumor development in a manner with that occurring following reduction of ^{V599E}B-Raf expression. Points, means from six different tumors; bars, ±SE.

FIG. 18: shows the region of Akt3 that causes preferential activation in melanoma. Activation is measured as the levels of phosphorylation; darker bands indicating higher activity. The lower bands indicate endogenous Akt activity. The domains of Akt3 were switched with those of Akt2 and constructs containing the chimeric constructs were nucleofected into the melanoma cell line WM35. Myristoylated Akt3 and Akt2 served as positive controls. Dead Akt3 (T305A/S472A) and Akt2 (T309A/S474A) served as negative controls. Transfer of wild type Akt3 led to increased activity in contrast to wild type Akt2 that did not. Constructs in which the pleckstrin homology (PH) domain from Akt3 (amino acids 1-110) was switched with those from Akt2 were used to identify the region of Akt3 leading to activation in melanoma cells. Note, only constructs containing the catalytic and regulatory (C/R) domains of Akt3 (from amino acids 111-497) led to activation. This maps the region from amino acids 111-497 as critical for activation of Akt3 in human melanomas. This is one site critical for therapeutic targeting that would specifically prevent Akt3 activation in melanomas. METHODS: HA-tagged wild type constructs, and chimeric constructs PH-Akt3-C/R-Akt2 and PH-Akt2-C/R-Akt3 were prepared by switching the pleckstrin homology (PH) domains (from amino acids 1-110) and catalytic domain (from 111-479 of Akt3 or 481 in Akt2). Constructs were nucleofected into the WM35 melanoma cell line using the Amaxa NHEM-NEO nucleofector reagent and 48 hours later analyzed by Western blot analysis by probing with an antibody to ser-473 of Akt.

### DETAILED DESCRIPTION OF THE PREFERRED EMBODIMENT

The present invention is directed in part to the discovery that Akt3 is an important serine/threonine protein kinase, and plays a role in melanoma survival so that melanoma tumor cells are resistant to apoptosis. A melanoma model that reflects the importance of Akt in melanoma tumorigenesis was used to identify Akt3 as the predominant isoform deregulated during melanoma tumorigenesis. As demonstrated herein the selective knockdown of Akt3, but not Akt1 or Akt2, decreases the level of total phosphorylated Akt and lowers the tumorigenic potential of melanoma cells. Consequently, Akt3 provides a therapeutic target for melanoma cancer.

The present invention is also directed in part to the discovery that V599E B-Raf plays a role in melanoma growth and proliferation. It has now been found that inhibition or reduction of B-Raf expression decreases tumor cells' proliferation and formation of new blood vessels (angiogenesis). It should be noted that due to errant sequence data the valine (V) to glutamic acid (E) substitution in B-Raf actually corresponds to codon 600 and the nucleotide 1799 (not 1796) in the correct version as shown in NCBI gene bank Accession Number. NT_{—}007914. Kumar et al., Clinical Cancer Research, 9: 3362-3368 (2003). However, in this application, we have used the uncorrected nucleotide and codon numbers throughout for historical and familiarity reasons.

The present inventors contemplate a combination therapy to treat tumor cells that involves the induction of apoptosis and reduction of cell proliferation and angiogenesis. In one embodiment, apoptosis is induced by reducing Akt3 activity and cell proliferation and angiogenesis is decreased by reducing V599E B-Raf activity. The present inventors also contemplate that reducing Akt3 activity in a tumor cell decreases the apoptotic threshold in tumor cells, especially in melanoma cells, allowing much lower doses of chemotherapy to be employed than based on conventional treatments. Thus, patients would receive a more effective treatment and experience less side effects from toxic chemotherapy drugs.

To aid in the understanding of the specification and claims, the following definitions are provided.

### DEFINITIONS

As used herein, the term “siRNA” means either: (i) a double stranded RNA oligonucleotide, or polynucleotide, that is 18 base pairs, 19 base pairs, 20 base pairs, 21 base pairs, 22 base pairs, 23 base pairs, 24 base pairs, 25 base pairs, 26 base pairs, 27 base pairs, 28 base pairs, 29 base pairs or 30 base pairs in length and that is capable of interfering with expression and activity of a Akt3 polypeptide, or a variant of the Akt3 polypeptide, wherein a single strand of the siRNA comprises a portion of a RNA polynucleotide sequence that encodes the Akt3 polypeptide, its variant, or a complementary sequence thereto; (ii) a single stranded oligonucleotide, or polynucleotide of 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides or 30 nucleotides in length and that is either capable of interfering with expression and/or activity of a target Akt3 polypeptide, or a variant of the Akt3 polypeptide, or that anneals to a complementary sequence to result in a dsRNA that is capable of interfering with target polypeptide expression, wherein such single stranded oligonucleotide comprises a portion of a RNA polynucleotide sequence that encodes the PTP-1B polypeptide, its variant, or a complementary sequence thereto; or (iii) an oligonucleotide, or polynucleotide, of either (i) or (ii) above wherein such oligonucleotide, or polynucleotide, has one, two, three or four nucleic acid alterations or substitutions therein.

“Nucleic acid or “polynucleotide” as used herein refers to purine- and pyrimidine-containing polymers of any length, either polyribonucleotides or polydeoxyribonucleotide or mixed polyribo-polydeoxyribonucleotides. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases.

A “gene” refers to an assembly of nucleotides that encode a polypeptide, and includes cDNA and genomic DNA nucleic acids.

A “vector” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. Viral vectors include retrovirus, adeno-associated virus, pox, baculovirus, vaccinia, herpes simplex, Epstein-Barr and adenovirus vectors. Non-viral vectors include, but are not limited to plasmids, liposomes, electrically charged lipids (cytofectins), DNA-protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. The transforming DNA can be integrated (covalently linked) into chromosomal DNA making up the genome of the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

The present invention contemplates isolation from melanoma of a gene encoding a human Akt3 protein or polypeptide of the invention, including a full length, or naturally occurring form of Akt3, and any human Akt3-specific antigenic fragments thereof. As used herein, “Akt3” refers to Akt3 polypeptide, and “akt3” refers to a gene encoding Akt3 polypeptide.

The term “Akt3” refers to Akt3 nucleic acid (DNA and RNA), protein (or polypeptide), their polymorphic variants, alleles, mutants, and interspecies homologs that have (i) substantial nucleotide sequence homology with the nucleotide sequence of the Accession Number AJ245709 (Homo sapiens mRNA for serine/threonine kinase Akt-3 (Akt3 gene)gi|5804885|emb|AJ245709.1|HSA245709[5804885]); Accession Number AF135794 (Homo sapiens AKT3 protein kinase mRNA, complete cds gi|4574743|gb|AF135794.1|AF135794[4574743]); Accession Number NM_{—}005465 (Homo sapiens v-akt murine thymoma viral oncogene homolog 3 (protein kinase B, gamma) (AKT3), transcript variant 1, mRNA gi|32307164|ref|NM_{—}005465.3|[32307164]); Accession Number NM_{—}181690 (Homo sapiens v-akt murine thymoma viral oncogene homolog 3 (protein kinase B, gamma) (AKT3), transcript variant 2, mRNA gi|32307162|ref|NM_{—}181690.1|[32307162]); Accession Number AY005799 (Homo sapiens protein kinase B gamma I (AKT3) mRNA, complete cds, alternatively spliced gi|15072339|gb|AY005799.1|[15072339]); Accession Number AF124141 (Homo sapiens protein kinase B gamma mRNA, complete cds gi|4757578|gb|AF124141.1|AF124141[4757578]); or (ii) substantial sequence homology with the encoding amino acid sequence Accession Number CAB53537 (Akt-3 protein [Homo sapiens] gi|5804886|emb|CAB53537.1|[5804886]); Accession Number AAD24196 (AKT3 protein kinase [Homo sapiens] gi|4574744|gb|AAD24196.1|AF135794_{—}1[4574744]); Accession Number AAF91073 (protein kinase B gamma 1 [Homo sapiens] gi|15072340|gb|AAF91073.1|[15072340]); Accession Number AAD29089 (protein kinase B gamma [Homo sapiens] gi|4757579|gb|AAD29089.1|AF124141_{—}1[4757579]); Accession Number NP_{—}005456 (v-akt murine thymoma viral oncogene homolog 3 isoform 1; protein kinase B gamma; RAC-gamma serine/threonine protein kinase; serine threonine protein kinase, Akt-3 [Homo sapiens] gi|4885549|ref|NP_{—}005456.1|[4885549]); Accession Number NP 859029 (v-akt murine thymoma viral oncogene homolog 3 isoform 2; protein kinase B gamma; RAC-gamma serine/threonine protein kinase; serine threonine protein kinase, Akt-3 [Homo sapiens] gi|32307163|ref|NP_{—}859029.1|[32307163]).

The term “B-Raf” refers to B-Raf nucleic acid (DNA and RNA), protein (or polypeptide), their polymorphic variants, alleles, mutants, and interspecies homologs that have (i) substantial nucleotide sequence homology with the nucleotide sequence of B-Raf found in Genbank (NM_{—}004333) a Homo sapiens v-raf murine sarcoma viral oncogene homolog B1 (BRAF), mRNA, gi|33188458|ref|NM_{—}004333.2|[33188458]. The cognate protein sequence for B-Raf is GenBank Accession Number P15056.

One B-Raf protein found in Genbank is M95712 Homo sapiens B-raf protein (BRAF) mRNA, complete cds gi|41387219|gb|M95712.2|HUMBRAF[41387219].

A “control sample” refers to a sample of biological material representative of healthy, cancer-free animals. The level of Akt3 or B-Raf in a control sample, or the encoding corresponding gene copy number, is desirably typical of the general population of normal, cancer-free subject of the same species. This sample either can be collected from an animal for the purpose of being used in the methods described in the present invention or it can be any biological material representative of normal, cancer-free animals obtained for other reasons but nonetheless suitable for use in the methods of this invention. A control sample can also be obtained from normal tissue from the animal that has cancer or is suspected of having cancer. A control sample also can refer to a given level of Akt3, representative of the cancer-free population, that has been previously established based on measurements from normal, cancer-free subjects. Alternatively, a biological control sample can refer to a sample that is obtained from a different individual or be a normalized value based on baseline data obtained from a population. Further, a control sample can be defined by a specific age, sex, ethnicity or other demographic parameters. In some situations, the control is implicit in the particular measurement. An example of an implicit control is where a detection method can only detect Akt3, or the corresponding gene copy number, when a level higher than that typical of a normal, cancer-free subject is present. A typical control level for a gene is two copies per cell. Another example is in the context of an immunohistochemical assay where the control level for the assay is known. Other instances of such controls are within the knowledge of the skilled person.

A level of Akt3 or B-Raf polypeptide or polynucleotide that is “expected” in a control sample refers to a level that represents a typical, cancer-free sample, and from which an elevated, or diagnostic, presence of Akt3 polypeptide or polynucleotide can be distinguished. Preferably, an “expected” level will be controlled for such factors as the age, sex, medical history, etc. of the mammal, as well as for the particular biological subject being tested.

The term “tumor cell” is meant a cell that is a component of a tumor in a subject, or a cell that is determined to be destined to become a component of a tumor, i.e., a cell that is a component of a precancerous lesion in a subject.

“cDNA” refers to complementary or copy DNA produced from an RNA template by the action of RNA-dependent DNA polymerase (reverse transcriptase). Thus, a “cDNA clone” means a duplex DNA sequence complementary to an RNA molecule of interest, carried in a cloning vector or PCR amplified. This term includes genes from which the intervening sequences have been removed.

“Cloning vector” refers to a plasmid or phage DNA or other DNA sequence that is able to replicate in a host cell. The cloning vector is characterized by one or more endonuclease recognition sites at which such DNA sequences may be cut in a determinable fashion without loss of an essential biological function of the DNA, which may contain a marker suitable for use in the identification of transformed cells.

“Expression vector” refers to a vehicle or vector similar to a cloning vector but which is capable of expressing a nucleic acid sequence that has been cloned into it, after transformation into a host. A nucleic acid sequence is “expressed” when it is transcribed to yield an mRNA sequence. In most cases, this transcript will be translated to yield amino acid sequence. The cloned gene is usually placed under the control of (i.e., operably linked to) an expression control sequence.

“Expression control sequence” or “regulatory sequence” refers to a nucleotide sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Expression control sequences will vary depending on whether the vector is designed to express the operably linked gene in a prokaryotic or eukaryotic host, and may contain transcriptional elements such as enhancer elements, termination sequences, tissue-specificity elements or translational initiation and termination sites.

“Operably linked” means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

“Host” means eukaryotes. The term includes an organism or cell that is the recipient of a replicable expression vector.

The introduction of the nucleic acids into the host cell by any method known in the art, including those described herein, will be referred to herein as “transformation.” The cells into which have been introduced nucleic acids described above are meant to also include the progeny of such cells.

Nucleic acids referred to herein as “isolated” are nucleic acids separated away from the nucleic acids of the genomic DNA or cellular RNA of their source of origin (e.g., as it exists in cells or in a mixture of nucleic acids such as a library), and may have undergone further processing. “Isolated”, as used herein, refers to nucleic or amino acid sequences that are at least 60% free, preferably 75% free, and most preferably 90% free from other components with which they are naturally associated. “Isolated” nucleic acids (polynucleotides) include nucleic acids obtained by methods described herein, similar methods or other suitable methods, including essentially pure nucleic acids, nucleic acids produced by chemical synthesis, by combinations of biological and chemical methods, and recombinant nucleic acids which are isolated. Nucleic acids referred to herein as “recombinant” are nucleic acids which have been produced by recombinant DNA methodology, including those nucleic acids that are generated by procedures which rely upon a method of artificial replication, such as the polymerase chain reaction (PCR) or cloning into a vector using restriction enzymes. “Recombinant” nucleic acids are also those that result from recombination events that occur through the natural mechanisms of cells, but are selected for after the introduction to the cells of nucleic acids designed to allow or make probable a desired recombination event. Portions of the isolated nucleic acids which code for polypeptides having a certain function can be identified and isolated by, for example, the method of Jasin, M., et al., U.S. Pat. No. 4,952,501.

As used herein, the terms “protein” and “polypeptide” are synonymous. “Peptides” are defined as fragments or portions of polypeptides, preferably fragments or portions having at least one functional activity (e.g., proteolysis, adhesion, fusion, antigenic, or intracellular activity) as the complete polypeptide sequence.

The terms “patient” or “subject” are used interchangeably and refer to mammals such as human patients and non-human primates, as well as experimental animals such as rabbits, rats, and mice, and other animals.

“Biological sample” as used herein is a sample of biological tissue or fluid that contains Akt3 and/or B-Raf nucleic acids or polypeptides, e.g., of a melanoma cancer protein, polynucleotide or transcript. Such samples include, but are not limited to, tissue isolated from humans. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, preferably eukaryotes such as fungi, plants, insects, protozoa, birds, fish, reptiles, and preferably a mammal such as rat, mice, cow, dog, guinea pig, or rabbit, and most preferably a primate such as chimpanzees or humans.

Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” is understood as a cell having specific structural properties, lacking differentiation and being capable of invasion and metastasis. Examples of cancers are skin, kidney, colon, breast, prostate and liver cancer. (see DeVita, V. et al. (eds.), 2001, Cancer Principles and Practice of Oncology, 6th. Ed., Lippincott Williams & Wilkins, Philadelphia, Pa.; this reference is herein incorporated by reference in its entirety for all purposes).

The term “apoptosis” and “programmed cell death” (PCD) are used as synonymous terms and describe the molecular and morphological processes leading to controlled cellular self-destruction (see, e.g., Kerr J. F. R. et al., 1972, Br J. Cancer. 26:239-257). Apoptotic cell death can be induced by a variety of stimuli, such as ligation of cell surface receptors, starvation, growth factor/survival factor deprivation, heat shock, hypoxia, DNA damage, viral infection, and cytotoxic/chemotherapeutical agents. The apoptotic process is involved in embryogenesis, differentiation, proliferation/homoeostasis, removal of defect and therefore harmful cells, and especially in the regulation and function of the immune system. Thus, dysfunction or disregulation of the apoptotic program is implicated in a variety of pathological conditions, such as immunodeficiency, autoimmune diseases, neurodegenerative diseases, and cancer. Apoptotic cells can be recognized by stereotypical morphological changes: the cell shrinks, shows deformation and looses contact to its neighboring cells. Its chromatin condenses, and finally the cell is fragmented into compact membrane-enclosed structures, called “apoptotic bodies” which contain cytosol, the condensed chromatin, and organelles. The apoptotic bodies are engulfed by macrophages and thus are removed from the tissue without causing an inflammatory response. This is in contrast to the necrotic mode of cell death in which case the cells suffer a major insult, resulting in loss of membrane integrity, swelling and disrupture of the cells. During necrosis, the cell contents are released uncontrolled into the cell's environment what results in damage of surrounding cells and a strong inflammatory response in the corresponding tissue. See, e.g., Tomei L. D. and Cope F. O., eds., 1991, Apoptosis: The Molecular Basis of Cell Death, Plainville, N.Y.: Cold Spring Harbor Laboratory Press; Isaacs J. T., 1993, Environ Health Perspect. 101(suppl 5):27-33; each of which is herein incorporated by reference in its entirety for all purposes. A variety of apoptosis assays are well known to one of skill in the art (e.g., DNA fragmentation assays, radioactive proliferation assays, DNA laddering assays for treated cells, Fluorescence microscopy of 4′-6-Diamidino-2-phenylindole (DAPI) stained cells assays, and the like).

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. See, for example, Davis et al., Basic Methods in Molecular Biology” Appleton & Lange, Norwalk, Conn. (1994). Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, 1984, Proteins).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 86%, 87%, 88%, 89%, 90%, 91%, 92%, 93%, 94%, 95%, 96%, 97%, 98%, 99%, or higher identity over a specified region (e.g., the sequence of the melanoma-associated Akt3 gene), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to the compliment of a test sequence. The definition also includes sequences that have deletions and/or additions, as well as those that have substitutions. As described below, the preferred algorithms can account for gaps and the like. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1991, Adv. Appi. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement).

Another example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, 1993, “Overview of principles of hybridization and the strategy of nucleic acid assays” in Techniques in Biochemistry and Molecular Biology—Hybridization with Nucleic Probes. Generally, stringent conditions are selected to be about 5-10° C. lower than the thermal melting point (T_{M}) for the specific sequence at a defined ionic strength pH. The T_{M} is the temperature (under defined ionic strength, pH, and nucleic concentration) at which 50% of the probes complementary to the target hybridize to the target sequence at equilibrium (as the target sequences are present in excess, at T_{M}, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Such washes can be performed for 5, 15, 30, 60, 120, or more minutes. For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and II; M. J. Gait (ed), 1984, Oligonucleotide Synthesis; B. D. Hames and S. J. Higgins (eds), 1984, Nucleic Acid Hybridization; Wu and Grossman (eds), Methods in Enzymology (Academic Press, Inc.), Vol. 154 and Vol. 155; Quirke and Taylor (eds), 1991, PCR-A Practical Approach; Hames and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning; J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology: Tools for Genomic and Molecular Research, American Society for Microbiology, Washington, D.C.

The term “reduces Akt3 activity” is used herein to refer to about a 25% to about a 100% decrease in Akt3 activity. The invention contemplates the inhibition Akt3 via any (a) agent that reduces the level of Akt3 mRNA or the level of Akt3 protein produced by the cell when the agent is administered to the cell or (b) any agent that affects the level of Akt3 mRNA or protein via the PI3K/Akt signal transduction pathway resulting a reduction in the level of Akt3 mRNA or the level of Akt3 protein produced by the cell when the agent is administered to the cell, or (c) any agent that decreases the activity of Akt3, such as through phosphorylation or dephosphorylation. Agents that decrease activity of downstream pathways that remove products of Akt3 activity and decreasing activity of upstream pathways providing reactants for Akt3 are also within the scope of this term. A decrease or change in Akt3 activity can be measured by any known method including, but not limited to, kinase assays, phosphorylation status in western blots, or levels of protein expression.

The term “reduces V599E B-Raf activity” is used herein to refer to about a 25% to about a 100% decrease in B-Raf activity. The invention contemplates the inhibition B-Raf via any (a) agent that reduces the level of V599E B-Raf mRNA or the level of V599E protein produced by the cell when the agent is administered to the cell or (b) any agent that affects the level of B-Raf mRNA or protein via the MAPK or ERK signal transduction pathway resulting a reduction in the level of V599E B-Raf mRNA or the level of V599E protein produced by the cell when the agent is administered to the cell, or (c) any agent that decreases the activity of B-Raf, such as through phosphorylation or dephosphorylation. Agents that decrease activity of downstream pathways that remove products of V599E B-Raf activity and decreasing activity of upstream pathways providing reactants for V599E B-Raf are also within the scope of this term. A decrease or change in B-Raf activity can be measured by any known method including, but not limited to, kinase assays, phosphorylation status in western blots, or levels of protein expression.

The term “treating a melanoma” refers to prohibiting, alleviating, ameliorating, halting, restraining, slowing or reversing the progression, or reducing tumor development in mammals and increasing apoptosis rates or inducing apoptosis in a tumor cell.

As used herein, the term “angiogenesis” when used in reference to reducing vascularization when, means that the amount of new blood vessel formation that occurs in the presence of an agent is decreased below the amount of blood vessel formation that occurs in the absence of an exogenously added agent. Methods for determining an amount of blood vessel formation in a tissue, including the immunohistochemical methods are well known in the art by quantifying the number of vessels staining positive for the CD-31 antigen or area in the tumor occupied by CD-31 positive vessels.

Detection of Akt3 and/or B-Raf Nucleic Acids

In some embodiments of the present invention, nucleic acids encoding an Akt3 or B-Raf polypeptide, including a full-length Akt3 or B-Raf protein, or any derivative, variant, homolog, or fragment thereof derived from a melanoma cell, will be used. Such nucleic acids are useful for any of a number of applications, including for the production of Akt3 or B-Raf protein, for diagnostic assays, for therapeutic applications, for Akt3-specific or B-Raf-specific probes, for assays for Akt3 or B-Raf binding and/or modulating compounds, to identify and/or isolate Akt3 or B-Raf homologs from other species or from mice, and other applications.

A. General Recombinant DNA Methods

Numerous applications of the present invention involve the cloning, synthesis, maintenance, mutagenesis, and other manipulations of nucleic acid sequences that can be performed using routine techniques in the field of recombinant genetics. Basic texts disclosing the general methods of use in this invention include Sambrook et al., Molecular Cloning, a Laboratory Manual (2n Ed. 1989); Kriegler, 1990, Gene Transfer and Expression: a Laboratory Manual; and Current Protocols in Molecular Biology, 1995, (Ausubel et al., eds.).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, 1981, Tetrahedron Letts. 22:1859-1862, using an automated synthesizer, as described in Van Devanter et al., 1984, Nucleic Acids Res. 12:6159-6168. Purification of oligonucleotides is by either native acrylamide gel electrophoresis or by anion-exchange HPLC as described in Pearson & Reanier, 1983, J. Chrom. 255:137-149.

The sequence of the cloned genes and synthetic oligonucleotides can be verified after cloning using, e.g., the chain termination method for sequencing double-stranded templates of Wallace et al., 1981, Gene 16:21-26.

B. Isolating and Detecting Akt3 and/or B-Raf Nucleotide Sequences

In some embodiments of the present invention, Akt3 and/or B-Raf nucleic acids will be isolated and cloned using recombinant methods. Such embodiments are used, e.g., to isolate Akt3 and/or B-Raf polynucleotides for protein expression or during the generation of variants, derivatives, expression cassettes, or other sequences derived from Akt3 and/or B-Raf, to monitor Akt3 and/or B-Raf gene expression, for the determination of Akt3 and/or B-Raf sequences in various species, for diagnostic purposes in a patient, i.e., to detect mutations in Akt3 and/or B-Raf, or for genotyping and/or forensic applications.

Polymorphic variants, alleles, and interspecies homologs and nucleic acids that are substantially identical to the Akt3 or B-Raf gene can be isolated using Akt3 or B-Raf nucleic acid probes, and oligonucleotides by screening libraries under stringent hybridization conditions. Alternatively, expression libraries can be used to clone Akt3 or B-Raf proteins, polymorphic variants, alleles, and interspecies homologs, by detecting expressed homologs immunologically with antisera or purified antibodies made against an Akt3 or B-Raf polypeptide, which also recognize and selectively bind to the Akt3 or B-Raf homolog.

To make a Akt3 cDNA library, one should choose a source that is rich in Akt3 RNA. To make a B-Raf cDNA library, one should choose a source that is rich in B-Raf RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, 1983, Gene 25:263-269; Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, 1977, Science 196:180-182. Colony hybridization is carried out as generally described in Grunstein et al., 1975, Proc. Natl. Acad. Sci. USA., 72:3961-3965.

More distantly related Akt3 or B-Raf homologs can be identified using any of a number of well known techniques, including by hybridizing an Akt3 probe or a B-Raf probe with a genomic or cDNA library using moderately stringent conditions, or under low stringency conditions using probes from regions which are selective for Akt3 or B-Raf, e.g., specific probes generated to the C-terminal domain. Also, a distant homolog can be amplified from a nucleic acid library using degenerate primer sets, i.e., primers that incorporate all possible codons encoding a given amino acid sequence, in particular based on a highly conserved amino acid stretch. Such primers are well known to those of skill, and numerous programs are available, e.g., on the internet, for degenerate primer design.

In certain embodiments, Akt3 or B-Raf polynucleotides will be detected using hybridization-based methods to determine, e.g., Akt3 or B-Raf RNA levels or to detect particular DNA sequences, e.g., for diagnostic purposes. For example, gene expression of Akt3 and/or B-Raf can be analyzed by techniques known in the art, e.g., Northern blotting, reverse transcription and PCR amplification of mRNA, including quantitative PCR analysis of mRNA levels with real-time PCR procedures (e.g., reverse transcriptase-TAQMAN™ amplification), dot blotting, in situ hybridization, RNase protection, probing DNA microchip arrays, and the like.

In another embodiment, high density oligonucleotide analysis technology (e.g., GeneChip™) may be used to identify orthologs, alleles, conservatively modified variants, and polymorphic variants of Akt3 and/or B-Raf, or to monitor levels of Akt3 and/or B-Raf mRNA. In the case where a homologs is linked to a known disease, e.g., melanoma, they can be used with GeneChip™ as a diagnostic tool in detecting melanoma in a biological sample, see, e.g., Gunthand et al., 1998, AIDS Res. Hum. Retroviruses 14:869-876; Kozal et al., 1996, Nat. Med. 2:753-759; Matson et al., 1995, Anal. Biochem. 224:110-106; Lockhart et al., 1996, Nat. Biotechnol. 14:1675-1680; Gingeras et al., 1998, Genome Res. 8:435-448; Hacia et al., 1998, Nucleic Acids Res. 26:3865-3866.

Detection of Akt3 and/or B-Raf polynucleotides and polypeptides can involve quantitative or qualitative detection of the polypeptide or polynucleotide, and can involve an actual comparison with a control value or, alternatively, can be performed so that the detection itself inherently indicates an increased level of Akt3 and/or B-Raf.

In certain embodiments, for example, diagnosis of melanoma cancer, the level of Akt3 and/or B-Raf polynucleotide, polypeptide, or protein activity will be quantified. In such embodiments, the difference between an elevated level of Akt3 and/or B-Raf and a normal, control level will preferably be statistically significant. Typically, a diagnostic presence, i.e., overexpression or an increase of Akt3 and/or B-Raf polypeptide or nucleic acid, represents at least about a 1.5, 2, 3, 5, 10, or greater fold increase in the level of Akt3 and/or B-Raf polypeptide or polynucleotide in the biological sample compared to a level expected in a noncancerous sample. Detection of Akt3 and/or B-Raf can be performed in vitro, i.e., in cells within a biological sample taken from the patient, or in vivo. In one embodiment an increased level of Akt3 and/or B-Raf is used as a diagnostic marker of Akt3 and/or B-Raf respectively. As used herein, a “diagnostic presence” indicates any level of Akt3 or B-Raf that is greater than that expected in a noncancerous sample. In a one embodiment, assays for an Akt3 or B-Raf polypeptide or polynucleotide in a biological sample are conducted under conditions wherein a normal level of Akt3 or B-Raf polypeptide or polynucleotide, i.e., a level typical of a noncancerous sample, i.e., cancer-free, would not be detected. In such assays, therefore, the detection of any Akt3 and/or B-Raf polypeptide or nucleic acid in the biological sample indicates a diagnostic presence, or increased level.

As described below, any of a number of methods to detect Akt3 and/or B-Raf can be used. An Akt3 and/or B-Raf polynucleotide level can be detected by detecting any cognate Akt3 or B-Raf DNA or RNA, including Akt3 genomic DNA, mRNA, and cDNA. An Akt3 or B-Raf polypeptide can be detected by detecting an Akt3 and/or B-Raf polypeptide itself, or by detecting Akt3 and/or B-Raf protein activity. Detection can involve quantification of the level of Akt3 and/or B-Raf (e.g., genomic DNA, cDNA, mRNA, or protein level, or protein activity) or, alternatively, can be a qualitative assessment of the level, or of the presence or absence, of Akt3 and/or B-Raf, in particular in comparison with a control level. Any of a number of methods to detect any of the above can be used, as described infra. Such methods include, for example, hybridization, amplification, and other assays.

In certain embodiments, the ability to detect an increased level, or diagnostic presence, in a cell is used as a marker for cancer cells, i.e., to monitor the number or localization of cancer cells in a patient, as detected in vivo or in vitro.

Typically, the Akt3 polynucleotides or polypeptides detected herein will be at least about 70% identical, and preferably 75%, 80%, 85%, 86%, 87%, 88%, 89%, 90%, 91%, 92%, 93%, 94%, 95%, 96%, 97%, 98%, 99% or more identical, over a region of at least about 50, 100, 200, or more nucleotides, or 20, 50, 100, or more amino acids, to the naturally occurring Akt3 gene. Such polynucleotides or polypeptides can represent functional or nonfunctional forms of Akt3, or any variant, derivative, or fragment thereof.

1. Detection of Copy Number

In one embodiment, e.g., for the diagnosis or presence of cancer, the copy number, i.e., the number of Akt3 genes in a cell, is evaluated. Generally, for a given autosomal gene, an animal has two copies of each gene. The copy number can be increased, however, by gene amplification or duplication, e.g., in cancer cells, or reduced by deletion. Methods of evaluating the copy number of a particular gene are well known to those of skill in the art, and include, inter alia, hybridization- and amplification-based assays.

a) Hybridization-Based Assays

Any of a number of hybridization-based assays can be used to detect the Akt3 gene or the copy number of Akt3 genes in the cells of a biological sample. One such method is by Southern blot. In a Southern blot, genomic DNA is typically fragmented, separated electrophoretically, transferred to a membrane, and subsequently hybridized to an Akt3-specific probe. For copy number determination, comparison of the intensity of the hybridization signal from the probe for the target region with a signal from a control probe for a region of normal genomic DNA (e.g., a nonamplified portion of the same or related cell, tissue, organ, and the like) provides an estimate of the relative Akt3 copy number. Southern blot methodology is well known in the art and is described, e.g., in Ausubel et al., or Sambrook et al., supra.

An alternative means for determining the copy number of Akt3 genes in a sample is by in situ hybridization, e.g., fluorescence in situ hybridization, or FISH. In situ hybridization assays are well known (e.g., Angerer, 1987, Meth. Enzymol 152:649). Generally, in situ hybridization comprises the following major steps:(1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments.

The probes used in such applications are typically labeled, e.g., with radioisotopes or fluorescent reporters. Preferred probes are sufficiently long, e.g., from about 50, 100, or 200 nucleotides to about 1000 or more nucleotides, so as to specifically hybridize with the target nucleic acid(s) under stringent conditions.

The present invention contemplates “comparative probe” methods, such as comparative genomic hybridization (CGH), are used to detect Akt3 gene amplification. In comparative genomic hybridization methods, a “test” collection of nucleic acids is labeled with a first label, while a second collection (e.g., from a healthy cell or tissue) is labeled with a second label. The ratio of hybridization of the nucleic acids is determined by the ratio of the first and second labels binding to each fiber in an array. Differences in the ratio of the signals from the two labels, e.g., due to gene amplification in the test collection, is detected and the ratio provides a measure of the Akt3 gene copy number.

Hybridization protocols suitable for use with the methods of the invention are described, e.g., in Albertson, 1984, EMBO J. 3:1227-1234; Pinkel, 1988, Proc. Natl. Acad. Sci. USA 85:9138-9142; EPO Pub. No. 430,402; Methods in Molecular Biology, Vol. 33: In Situ Hybridization Protocols, Choo, Ed., 1994, Humana Press, Totowa, N.J., and the like.

b) Amplification-Based Assays

In another embodiment, amplification-based assays are used to detect Akt3 or to measure the copy number of Akt3 genes. In such assays, the Akt3 nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the copy number of the Akt3 gene. Methods of quantitative amplification are well known to those of skill in the art. Detailed protocols for quantitative PCR are provided, e.g., in Innis et al., 1990, PCR Protocols: A Guide to Methods and Applications, Academic Press, Inc. N.Y.). The nucleic acid sequence for Akt3 is sufficient to enable one of skill to routinely select primers to amplify any portion of the gene.

In some embodiments, a TaqMan based assay is used to quantify Akt3 polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer.

Other suitable amplification methods which are contemplated by the invention include, but are not limited to, ligase chain reaction (LCR) (see, Wu and Wallace, 1989, Genomics 4:560, Landegren et al., 1988, Science 241:1077, and Barringer et al., 1990, Gene 89:117), transcription amplification (Kwok et al., 1989, Proc. Natl. Acad. Sci. USA 86:1173), self-sustained sequence replication (Guatelli et al., 1990, Proc. Nat. Acad. Sci. USA 87:1874), dot PCR, and linker adapter PCR, etc.

2. Detection of Akt3 and/or B-Raf Expression

a) Direct Hybridization-Based Assays

Methods of detecting and/or quantifying the level of Akt3 and/or B-Raf gene transcripts (mRNA or cDNA made there from) using nucleic acid hybridization techniques are known to those of skill in the art (see, Sambrook et al., 1989, Molecular Cloning: A Laboratory Manual, 2D Ed., Vols 1-3, Cold Spring Harbor Press, New York).

For example, one method for evaluating the presence, absence, or quantity of Akt3 cDNA involves a Northern blot. In brief, in a typical embodiment, mRNA is isolated from a given biological sample, electrophoresed to separate the mRNA species, and transferred from the gel to a nitrocellulose membrane. Labeled Akt3 probes are then hybridized to the membrane to identify and/or quantify the mRNA.

b) Amplification-Based Assays

In another embodiment, an Akt3 and/or B-Raf transcript (e.g., Akt3 mRNA) is detected using amplification-based methods (e.g., RT-PCR). RT-PCR methods are well known to those of skill (see, e.g., Ausubel et al., supra). Preferably, quantitative RT-PCR is used, thereby allowing the comparison of the level of mRNA in a sample with a control sample or value.

3. Detection of Akt3 and/or B-Raf Polypeptide Expression

In addition to the detection of Akt3 and/or B-Raf genes and gene expression using nucleic acid hybridization technology, Akt3 and/or B-Raf levels can also be detected and/or quantified by detecting or quantifying the polypeptide. Akt3 or B-Raf polypeptides are detected and quantified by any of a number of means well known to those of skill in the art. These include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. Akt3 polypeptide detection is discussed infra.

C. Expression in Prokaryotes and Eukaryotes

In some embodiments, it is desirable to produce Akt3 and/or B-Raf polypeptides using recombinant technology. To obtain high level expression of a cloned gene or nucleic acid, such as a cDNA encoding an Akt3 or B-Raf polypeptide, an Akt3 or B-Raf sequence is typically subcloned into an expression vector that contains a strong promoter to direct transcription, a transcription/translation terminator, and if for a nucleic acid encoding a protein, a ribosome binding site for translational initiation. Suitable bacterial promoters are well known in the art and are described, e.g., in Sambrook et al. and Ausubel et al. Bacterial expression systems for expressing the Akt3 protein are available in, e.g., E. coli, Bacillus sp., and Salmonella (Palva et al., 1983, Gene 22:229-235; Mosbach et al., 1983, Nature 302:543-545. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

For therapeutic applications, Akt3 and/or B-Raf nucleic acids are introduced into a cell, in vitro, in vivo, or ex vivo, using any of a large number of methods including, but not limited to, infection with viral vectors, liposome-based methods, biolistic particle acceleration (the gene gun), and naked DNA injection. Such therapeutically useful nucleic acids include, but are not limited to, coding sequences for full-length Akt3 or B-Raf, coding sequences for a Akt3 or B-Raf fragment, domain, derivative, or variant, Akt3 or B-Raf antisense sequences, Akt3 or B-Raf siRNA sequences, and Akt3 or B-Raf ribozymes. Typically, such sequences will be operably linked to a promoter, but in numerous applications a nucleic acid will be administered to a cell that is itself directly therapeutically effective, e.g., certain antisense, siRNA, or ribozyme molecules.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the Akt3-encoding or B-Raf-encoding nucleic acid in host cells. A typical expression cassette thus contains a promoter operably linked to the nucleic acid sequence encoding an Akt3 or B-Raf polypeptide, and signals required for efficient polyadenylation of the transcript, ribosome binding sites, and translation termination. The nucleic acid sequence encoding an Akt3 or B-Raf polypeptide can be linked to a cleavable signal peptide sequence to promote secretion of the encoded protein by the transfected cell. Additional elements of the cassette can include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region can be obtained from the same gene as the promoter sequence or can be obtained from different genes.

The particular expression vector used to transport the genetic information into the cell is not particularly critical. Any of the conventional vectors used for expression in eukaryotic or prokaryotic cells can be used. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., 1988, Gene 67:31-40), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. For example, mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaL SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaL SbaI, EcoRI, and BelI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive Rous Sarcoma Virus Long Terminal Repeat (RSV-LTR) promoter, hygromycin selectable marker; Invitrogen), pCEP4 (BamH1, SfiI, XhoI, NotI, NheI, HindIII, NheI, PvuII, and KpnI cloning site, constitutive human cytomegalovirus (hCMV) immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include, but are limited to, pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) contemplated by this invention include but are not limited to pSC11 (Sinal cloning site, TK- and (3-gal selection), pMJ601 (SalI, SmaI, AflI, NarI, BspMII, BamHI, ApaI, NheI, SacII, KpnI, and HindIII cloning site; TK- and beta ((3)-gal selection), and pTKgptF1S (EcoRI, PstI, SalI, AccI, HindIII, SbaI, BamHII, and Hpa cloning site, TK or XPRT selection).

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Once a particular recombinant DNA molecule is identified and isolated, several methods known in the art may be used to propagate it. Once a suitable host system and growth conditions are established, recombinant expression vectors can be propagated and prepared in quantity. The expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few and which are known to those of skill in the art.

In addition, a host cell strain may be chosen which modulates the expression of the inserted sequences, or modifies and processes the gene product in the specific fashion desired. Different host cells have characteristic and specific mechanisms for the translational and post-translational processing and modification of proteins. Appropriate cell lines or host systems can be chosen to ensure the desired modification and processing of the foreign protein expressed. Expression in yeast can produce a biologically active product. Expression in eukaryotic cells can increase the likelihood of “native” folding. Moreover, expression in mammalian cells can provide a tool for reconstituting, or constituting, Akt3 and/or B-Raf activity in melanoma. Furthermore, different vector/host expression systems may affect processing reactions, such as proteolytic cleavages, to a different extent.

Standard transfection methods are used to produce bacterial, mammalian, yeast or insect cell lines that express large quantities of a Akt3 or a B-Raf protein, which are then purified using standard techniques (see, e.g., Colley et al., 1989, J. Biol. Chem. 264:17619-17622; “Guide to Protein Purification,” in Methods in Enzymology, Vol. 182, 1990 (Deutscher, Ed.). Transformation of eukaryotic and prokaryotic cells are performed according to standard techniques (see, e.g., Morrison, 1977, J. Bact. 132:349-351; Clark-Curtiss & Curtiss, Methods in Enzymology 101:347-362, 1983 (Wu et al., eds.).

Any of the well known procedures for introducing foreign nucleotide sequences into host cells can be used. These include the use of reagents such as Superfect (Qiagen), liposomes, calcium phosphate transfection, polybrene, protoplast fusion, electroporation, microinjection, plasmid vectors, viral vectors, biolistic particle acceleration (the gene gun), or any of the other well known methods for introducing cloned genomic DNA, cDNA, synthetic DNA or other foreign genetic material into a host cell (see, e.g., Sambrook et al., supra).

After the expression vector is introduced into the cells, the transfected cells are cultured under conditions favoring expression of the Akt3 and/or B-Raf polypeptide, which is recovered from the culture using standard techniques identified below. Methods of culturing prokaryotic or eukaryotic cells are well known and are taught, e.g., in Ausubel et al., Sambrook et al., and in Freshney, 1993, Culture of Animal Cells, 3.sup.rd. Ed., A Wiley-Liss Publication.

Any of the well known procedures for introducing foreign nucleotide sequences into host cells can be used to introduce a vector, e.g., a targeting vector, into cells. Any of the well known procedures for introducing foreign nucleotide sequences into host cells can be used. As provided infra, nucleic acids of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., 62, 63). The recombinant retrovirus can then be used to infect and thereby deliver to the infected cells nucleic acids. The exact method of introducing the nucleic acid into mammalian cells is, of course, not limited to the use of retroviral vectors. Other techniques are widely available for this procedure including the use of adenoviral vectors, adeno-associated viral (AAV) vectors, lentiviral vectors, pseudotyped retroviral vectors. Physical transduction techniques can also be used, such as liposome delivery and receptor-mediated and other endocytosis mechanisms. This invention can be used in conjunction with any of these or other commonly used gene transfer methods.

### Inducing Apoptosis in a Cancer Cell by Reducing Akt3 Activity Levels in Cells

In one embodiment, this invention provides methods of inducing apoptosis in a melanoma tumor cell by contacting a cell with an agent that reduces Akt3 activity. In a preferred embodiment, the agent is a siRNA molecule.

A siRNA polynucleotide is a RNA nucleic acid molecule that mediates the effect of RNA interference, a post-transcriptional gene silencing mechanism. A siRNA polynucleotide preferably comprises a double-stranded RNA (dsRNA) but is not intended to be so limited and may comprise a single-stranded RNA (see, e.g., Martinez et al. Cell 110:563-74 (2002)). A siRNA polynucleotide may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of an embodiment of the subject invention siRNA polynucleotides are also intended to describe the corresponding RNA sequences and their complements, given the well established principles of complementary nucleotide base-pairing. A siRNA may be transcribed using as a template a DNA (genomic, cDNA, or synthetic) that contains a RNA polymerase promoter, for example, a U6 promoter or the H1 RNA polymerase III promoter, or the siRNA may be a synthetically derived RNA molecule. In certain embodiments the subject invention siRNA polynucleotide may have blunt ends, that is, each nucleotide in one strand of the duplex is perfectly complementary (e.g., by Watson-Crick base-pairing) with a nucleotide of the opposite strand. In certain other embodiments, at least one strand of the subject invention siRNA polynucleotide has at least one, and preferably two nucleotides that “overhang” (i.e., that do not base pair with a complementary base in the opposing strand) at the 3′ end of either strand, or preferably both strands, of the siRNA polynucleotide. In certain other embodiments of the invention, each strand of the siRNA polynucleotide duplex has a two-nucleotide overhang at the 3′ end. The two-nucleotide overhang is preferably a thymidine dinucleotide (TT) but may also comprise other bases, for example, a TC dinucleotide or a TG dinucleotide, or any other dinucleotide. For a discussion of 3′ ends of siRNA polynucleotides see, e.g., WO 01/75164.

Preferred siRNA polynucleotides comprise double-stranded oligomeric nucleotides of about 18-30 nucleotide base pairs, preferably about 18, 19, 20, 21, 22, 23, 24, 25, 26, or 27 base pairs, and in other preferred embodiments about 19, 20, 21, 22 or 23 base pairs, or about 27 base pairs, whereby the use of “about” indicates, as described above, that in certain embodiments and under certain conditions the processive cleavage steps that may give rise to functional siRNA polynucleotides that are capable of interfering with expression of a selected polypeptide may not be absolutely efficient. Hence, siRNA polynucleotides, for instance, of “about” 18, 19, 20, 21, 22, 23, 24, or 25 base pairs may include one or more siRNA polynucleotide molecules that may differ (e.g., by nucleotide insertion or deletion) in length by one, two, three or four base pairs, by way of non-limiting theory as a consequence of variability in processing, in biosynthesis, or in artificial synthesis. The contemplated siRNA polynucleotides of the present invention may also comprise a polynucleotide sequence that exhibits variability by differing (e.g., by nucleotide substitution, including transition or transversion) at one, two, three or four nucleotides from a particular sequence, the differences occurring at any of positions 1, 2, 3, 4, 5, 6, 7, 8, 9, 10, 11, 12, 13, 14, 15, 16, 17, 18, or 19 of a particular siRNA polynucleotide sequence, or at positions 20, 21, 22, 23, 24, 25, 26, or 27 of siRNA polynucleotides depending on the length of the molecule, whether situated in a sense or in an antisense strand of the double-stranded polynucleotide. The nucleotide substitution may be found only in one strand, by way of example in the antisense strand, of a double-stranded polynucleotide, and the complementary nucleotide with which the substitute nucleotide would typically form hydrogen bond base pairing may not necessarily be correspondingly substituted in the sense strand. In preferred embodiments, the siRNA polynucleotides are homogeneous with respect to a specific nucleotide sequence. As described herein, preferred siRNA polynucleotides interfere with expression of the Akt3 polypeptide of the invention. These polynucleotides may also find uses as probes or primers.

Polynucleotides that are siRNA polynucleotides of the present invention may in certain embodiments be derived from a single-stranded polynucleotide that comprises a single-stranded oligonucleotide fragment (e.g., of about 18-30 nucleotides, which should be understood to include any whole integer of nucleotides including and between 18 and 30) and its reverse complement, typically separated by a spacer sequence. According to certain such embodiments, cleavage of the spacer provides the single-stranded oligonucleotide fragment and its reverse complement, such that they may anneal to form (optionally with additional processing steps that may result in addition or removal of one, two, three or more nucleotides from the 3′ end and/or the 5′ end of either or both strands) the double-stranded siRNA polynucleotide of the present invention. In certain embodiments the spacer is of a length that permits the fragment and its reverse complement to anneal and form a double-stranded structure (e.g., like a hairpin polynucleotide) prior to cleavage of the spacer (and, optionally, subsequent processing steps that may result in addition or removal of one, two, three, four, or more nucleotides from the 3′ end and/or the 5′ end of either or both strands). A spacer sequence may therefore be any polynucleotide sequence as provided herein that is situated between two complementary polynucleotide sequence regions which, when annealed into a double-stranded nucleic acid, comprise a siRNA polynucleotide. Preferably a spacer sequence comprises at least 4 nucleotides, although in certain embodiments the spacer may comprise 5, 6, 7, 8, 9, 10, 11, 12, 13, 14, 15, 16, 17, 18, 19, 20, 21-25, 26-30, 31-40, 41-50, 51-70, 71-90, 91-110, 111-150, 151-200 or more nucleotides. Examples of siRNA polynucleotides derived from a single nucleotide strand comprising two complementary nucleotide sequences separated by a spacer have been described (e.g., Brummelkamp et al., 2002 Science 296:550; Paddison et al., 2002 Genes Develop. 16:948; Paul et al. Nat. Biotechnol. 20:505-508 (2002); Grabarek et al., BioTechniques 34:734-44 (2003)).

Polynucleotide variants may contain one or more substitutions, additions, deletions, and/or insertions such that the activity of the siRNA polynucleotide is not substantially diminished, as described above. The effect on the activity of the siRNA polynucleotide may generally be assessed as described herein or using conventional methods. Variants preferably exhibit at least about 75%, 78%, 80%, 85%, 87%, 88% or 89% identity and more preferably at least about 90%, 92%, 95%, 96%, 97%, 98%, or 99% identity to a portion of a polynucleotide sequence that encodes a native Akt3. The percent identity may be readily determined by comparing sequences of the polynucleotides to the corresponding portion of a full-length Akt3 polynucleotide such as those known to the art and cited herein, using any method including using computer algorithms well known to those having ordinary skill in the art, such as Align or the BLAST algorithm (Altschul, J. Mol. Biol. 219:555-565, 1991; Henikoff and Henikoff, Proc. Natl. Acad. Sci. USA 89:10915-10919, 1992), which is available at the NCBI website. Default parameters may be used.

Certain siRNA polynucleotide variants are substantially homologous to a portion of a native PTP1B gene. Single-stranded nucleic acids derived (e.g., by thermal denaturation) from such polynucleotide variants are capable of hybridizing under moderately stringent conditions to a naturally occurring DNA or RNA sequence encoding a native Akt3 polypeptide (or a complementary sequence). A polynucleotide that detectably hybridizes under moderately stringent conditions may have a nucleotide sequence that includes at least 10 consecutive nucleotides, more preferably 11, 12, 13, 14, 15, 16, 17, 18, 19, 20, 21, 22, 23, 24, 25, 26, 27, 28, 29 or 30 consecutive nucleotides complementary to a particular polynucleotide. In certain preferred embodiments such a sequence (or its complement) will be unique to an Akt polypeptide for which interference with expression is desired, and in certain other embodiments the sequence (or its complement) may be shared by Akt3 and one or more Akt isoforms for which interference with polypeptide expression is desired. In certain preferred embodiments such a sequence (or its complement) will be unique to a B-Raf polypeptide for which interference with expression is desired, and in certain other embodiments the sequence (or its complement) may be shared by B-Raf and one or more Raf isoforms for which interference with polypeptide expression is desired.

Suitable moderately stringent conditions include, for example, pre-washing in a solution of 5×SSC, 0.5% SDS, 1.0 mM EDTA (pH 8.0); hybridizing at 50° C.-70° C., 5×SSC for 1-16 hours (e.g., overnight); followed by washing once or twice at 22-65° C. for 20-40 minutes with one or more each of 2×, 0.5× and 0.2×SSC containing 0.05-0.1% SDS. For additional stringency, conditions may include a wash in 0.1×SSC and 0.1% SDS at 50-60° C. for 15-40 minutes. As known to those having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used, and of the blotted, proband nucleic acid sample. Accordingly, it will be appreciated that suitably stringent conditions can be readily selected without undue experimentation when a desired selectivity of the probe is identified, based on its ability to hybridize to one or more certain proband sequences while not hybridizing to certain other proband sequences.

Sequence specific siRNA polynucleotides of the present invention may be designed using one or more of several criteria. For example, to design a siRNA polynucleotide that has 19 consecutive nucleotides identical to a sequence encoding a polypeptide of interest (e.g., Akt3 and other polypeptides described herein), the open reading frame of the polynucleotide sequence may be scanned for 21-base sequences that have one or more of the following characteristics: (1) an A+T/G+C ratio of approximately 1:1 but no greater than 2:1 or 1:2; (2) an AA dinucleotide or a CA dinucleotide at the 5′ end; (3) an internal hairpin loop melting temperature less than 55° C.; (4) a homodimer melting temperature of less than 37° C. (melting temperature calculations as described in (3) and (4) can be determined using computer software known to those skilled in the art); (5) a sequence of at least 16 consecutive nucleotides not identified as being present in any other known polynucleotide sequence (such an evaluation can be readily determined using computer programs available to a skilled artisan such as BLAST to search publicly available databases). Alternatively, a siRNA polynculeotide sequence may be designed and chosen using a computer software available commercially from various vendors (e.g., OligoEngine™ (Seattle, Wash.); Dharmacon, Inc. (Lafayette, Colo.); Ambion Inc. (Austin, Tex.); and QIAGEN, Inc. (Valencia, Calif.)). (See also Elbashir et al., Genes & Development 15:188-200 (2000); Elbashir et al., Nature 411:494-98 (2001). The siRNA polynucleotides may then be tested for their ability to interfere with the expression of the target polypeptide according to methods known in the art and described herein. The determination of the effectiveness of an siRNA polynucleotide includes not only consideration of its ability to interfere with polypeptide expression but also includes consideration of whether the siRNA polynucleotide manifests undesirably toxic effects, for example, apoptosis of a cell for which cell death is not a desired effect of RNA interference (e.g., interference of Akt3 expression in a cell).

Persons having ordinary skill in the art will also readily appreciate that as a result of the degeneracy of the genetic code, many nucleotide sequences may encode a polypeptide as described herein. That is, an amino acid may be encoded by one of several different codons and a person skilled in the art can readily determine that while one particular nucleotide sequence may differ from another (which may be determined by alignment methods disclosed herein and known in the art), the sequences may encode polypeptides with identical amino acid sequences. By way of example, the amino acid leucine in a polypeptide may be encoded by one of six different codons (TTA, TTG, CTT, CTC, CTA, and CTG) as can serine (TCT, TCC, TCA, TCG, AGT, and AGC). Other amino acids, such as proline, alanine, and valine, for example, may be encoded by any one of four different codons (CCT, CCC, CCA, CCG for proline; GCT, GCC, GCA, GCG for alanine; and GTT, GTC, GTA, GTG for valine). Some of these polynucleotides bear minimal homology to the nucleotide sequence of any native gene. Nonetheless, polynucleotides that vary due to differences in codon usage are specifically contemplated by the present invention.

Polynucleotides, including target polynucleotides (e.g., polynucleotides capable of encoding a target polypeptide of interest), may be prepared using any of a variety of techniques, which will be useful for the preparation of specifically desired siRNA polynucleotides and for the identification and selection of desirable sequences to be used in siRNA polynucleotides. For example, a polynucleotide may be amplified from cDNA prepared from a suitable cell or tissue type. Such polynucleotides may be amplified via polymerase chain reaction (PCR). For this approach, sequence-specific primers may be designed based on the sequences provided herein and may be purchased or synthesized. An amplified portion may be used to isolate a full-length gene, or a desired portion thereof, from a suitable library (e.g., human melanoma cDNA) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences. Suitable sequences for a siRNA polynucleotide contemplated by the present invention may also be selected from a library of siRNA polynucleotide sequences.

For hybridization techniques, a partial sequence may be labeled (e.g., by nick-translation or end-labeling with ³²P) using well known techniques. A bacterial or bacteriophage library may then be screened by hybridizing filters containing denatured bacterial colonies (or lawns containing phage plaques) with the labeled probe (see, e.g., Sambrook et al., Molecular Cloning: A Laboratory Manual, Cold Spring Harbor Laboratories, Cold Spring Harbor, N.Y., 2001). Hybridizing colonies or plaques are selected and expanded, and the DNA is isolated for further analysis. Clones may be analyzed to determine the amount of additional sequence by, for example, PCR using a primer from the partial sequence and a primer from the vector. Restriction maps and partial sequences may be generated to identify one or more overlapping clones. A full-length cDNA molecule can be generated by ligating suitable fragments, using well known techniques.

Alternatively, numerous amplification techniques are known in the art for obtaining a full-length coding sequence from a partial cDNA sequence. Within such techniques, amplification is generally performed via PCR. One such technique is known as “rapid amplification of cDNA ends” or RACE. This technique involves the use of an internal primer and an external primer, which hybridizes to a polyA region or vector sequence, to identify sequences that are 5′ and 3′ of a known sequence. Any of a variety of commercially available kits may be used to perform the amplification step. Primers may be designed using, for example, software well known in the art. Primers (or oligonucleotides for other uses contemplated herein, including, for example, probes and antisense oligonucleotides) are preferably 15, 16, 17, 18, 19, 20, 21, 22, 23, 24, 25, 26, 27, 28, 29, 30, 31 or 32 nucleotides in length, have a GC content of at least 40% and anneal to the target sequence at temperatures of about 54° C. to 72° C. The amplified region may be sequenced as described above, and overlapping sequences assembled into a contiguous sequence. Certain oligonucleotides contemplated by the present invention may, for some preferred embodiments, have lengths of 18, 19, 20, 21, 22, 23, 24, 25, 26, 27, 28, 29, 30, 31, 32, 33-35, 35-40, 41-45, 46-50, 56-60, 61-70, 71-80, 81-90 or more nucleotides.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives, and cosmids. Vectors of particular interest include expression vectors, replication vectors, probe generation vectors, and sequencing vectors. In general, a suitable vector contains an origin of replication functional in at least one organism, convenient restriction endonuclease sites, and one or more selectable markers. (See, e.g., WO 01/96584; WO 01/29058; U.S. Pat. No. 6,326,193; U.S. 2002/0007051). Other elements will depend upon the desired use, and will be apparent to those having ordinary skill in the art. For example, the invention contemplates the use of siRNA polynucleotide sequences in the preparation of recombinant nucleic acid constructs including vectors for interfering with the expression of a desired target polypeptide such as a Akt3 or B-Raf polypeptide in vivo; the invention also contemplates the generation of siRNA transgenic or “knock-out” animals and cells (e.g., cells, cell clones, lines or lineages, or organisms in which expression of one or more desired polypeptides (e.g., a target polypeptide) is fully or partially compromised). An siRNA polynucleotide that is capable of interfering with expression of a desired polypeptide (e.g., a target polypeptide) as provided herein thus includes any siRNA polynucleotide that, when contacted with a subject or biological source as provided herein under conditions and for a time sufficient for target polypeptide expression to take place in the absence of the siRNA polynucleotide, results in a statistically significant decrease (alternatively referred to as “knockdown” of expression) in the level of target polypeptide expression that can be detected. Preferably the decrease is greater than 10%, more preferably greater than 20%, more preferably greater than 30%, more preferably greater than 40%, 50%, 60%, 70%, 75%, 80%, 85%, 90%, 95% or 98% relative to the expression level of the polypeptide detected in the absence of the siRNA, using conventional methods for determining polypeptide expression as known to the art and provided herein. Preferably, the presence of the siRNA polynucleotide in a cell does not result in or cause any undesired toxic effects, for example, apoptosis or death of a cell in which apoptosis is not a desired effect of RNA interference.

Exemplary 19mer sequences for the Akt3 siRNA as disclosed herein are to human Akt3 (NM_{—}005465): Akt3 duplex 2:CUAUCUACAUUCCGGAAAG (SEQ ID NO:1); Akt3 duplex 4:GAAUUUACAGCUCAGACUA (SEQ ID NO:2); and Akt3 duplex 5:CAGCUCAGACUAUUACAAU (SEQ ID NO:3).

Exemplary 25mer sequences for the Akt3 siRNA as disclosed herein are as follows:

| Primer | |
|---|---|
| Name | Sequence |
| Akt3#2 | CUUGGACUAUCUACAUUCCGGAAAG (SEQ ID NO: 4) |
| Sense | |
| Akt3#2 | CUUUCCGGAAUGUAGAUAGUCCAAG (SEQ ID NO: 5) |
| Antisense | |
| Akt3#4 | GAUGAAGAAUUUACAGCUCAGACUA (SEQ ID NO: 6) |
| Sense | |
| Akt3#4 | UAGUCUGAGCUGUAAAUUCUUCAUC (SEQ ID NO: 7) |
| Antisense | |
| Akt3#5 | AAUUUACAGCUCAGACUAUUACAAU (SEQ ID NO: 8) |
| Sense | |
| Akt3#5 | AUUGUAAUAGUCUGAGCUGUAAAUU (SEQ ID NO: 9) |
| Antisense | |

Exemplary 25mer sequences for the B-Raf siRNA as disclosed herein are to human Mutant B-Raf: 5′ GGUCUAGCUACAGAGAAAUCUCGAU 3′ (SEQ ID NO:10) and to human wild-type B-Raf 5′ 5′ GGACAAAGAAUUGGAUCUGGAUCAU 3′ (SEQ ID NO:11).

The present invention also relates to use of a viral-mediated strategy that result in silencing of a targeted gene, PTEN, via siRNA. Use of this strategy results in markedly diminished expression of PTEN, thereby leading to an increase in total phosphorylated Akt. This viral-mediated strategy is useful in identifying the mechanism underlying Akt3 deregulation in melanomas in order to model biological processes or to provide therapy for this cancer.

The present invention also relates to vectors and to constructs that include or encode siRNA polynucleotides of the present invention, and in particular to “recombinant nucleic acid constructs” that include any nucleic acid such as a DNA polynucleotide segment that may be transcribed to yield Akt3 polynucleotide-specific siRNA polynucleotides according to the invention as provided above; to host cells which are genetically engineered with vectors and/or constructs of the invention and to the production of siRNA polynucleotides, polypeptides, and/or fusion proteins of the invention, or fragments or variants thereof, by recombinant techniques. SiRNA sequences disclosed herein as RNA polynucleotides may be engineered to produce corresponding DNA sequences using well-established methodologies such as those described herein. Thus, for example, a DNA polynucleotide may be generated from any siRNA sequence described herein, such that the present siRNA sequences will be recognized as also providing corresponding DNA polynucleotides (and their complements). These DNA polynucleotides are therefore encompassed within the contemplated invention, for example, to be incorporated into the subject invention recombinant nucleic acid constructs from which siRNA may be transcribed.

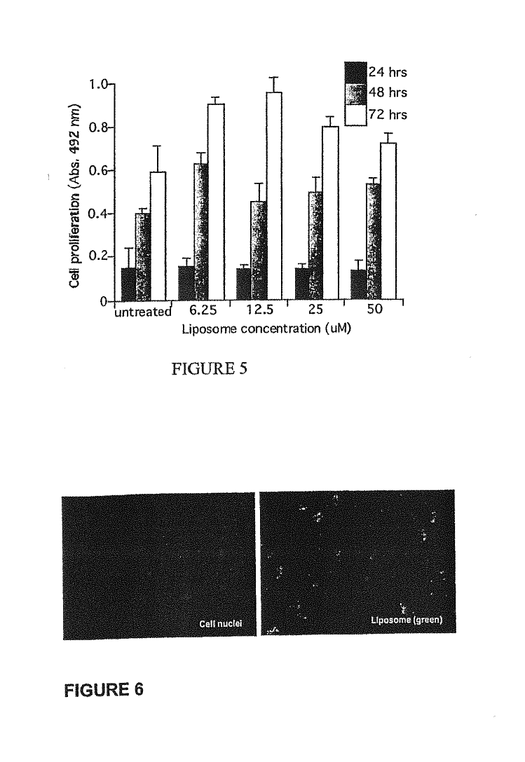

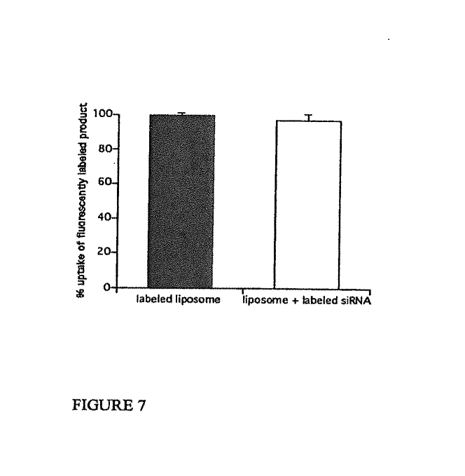

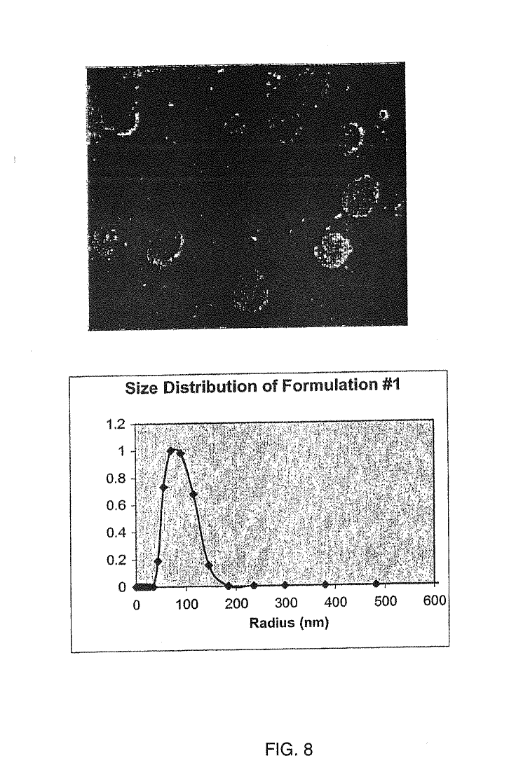

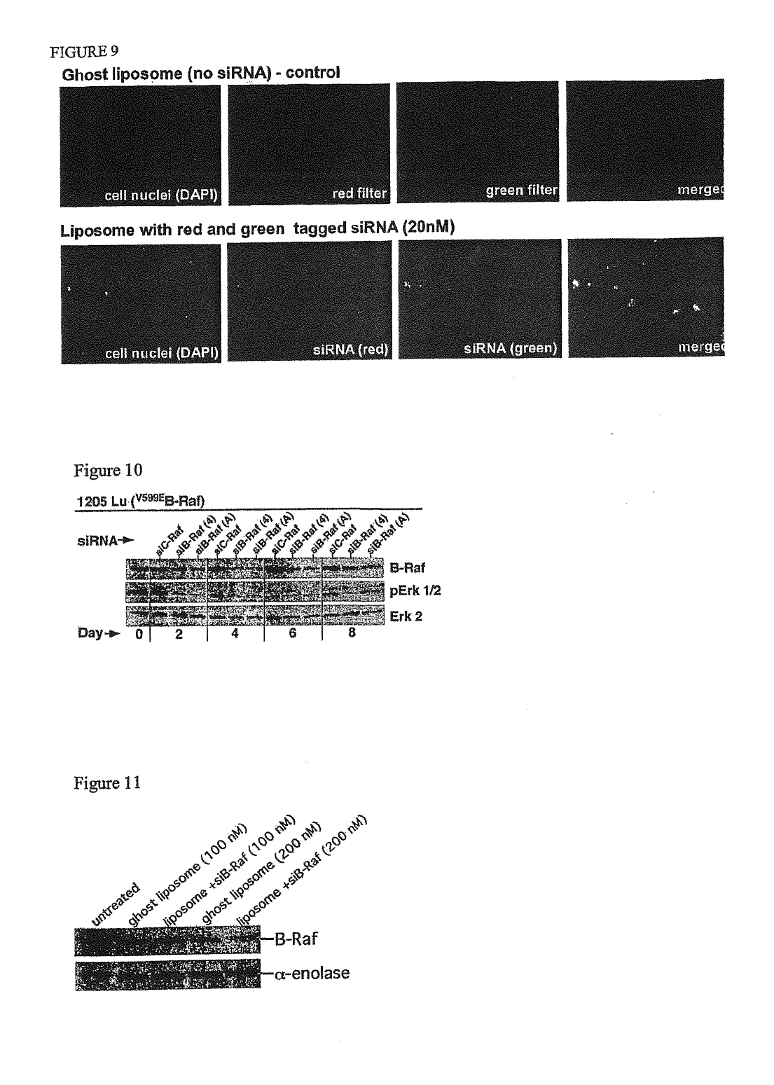

In an another embodiment, the agent is a siRNA molecule wherein the siRNA molecule comprises a polynucleotide having a sequence of 5′GGUCUAGCUACAGAGAAAUCUCGAU 3′ (SEQ ID NO:10) or the complement thereof. In yet another embodiment, the agent is a siRNA molecule wherein the siRNA molecule comprises a polynucleotide having a sequence of 5′ CUAUCUACAUUCCGGAAAG 3′(SEQ ID NO:1), or the complement thereof. In yet another embodiment, the agent is a siRNA molecule wherein the siRNA molecule comprises a polynucleotide having a sequence of 5′ GAAUUUACAGCUCAGACUA 3′ (SEQ ID NO:2), or the complement thereof. In still another embodiment, the agent is a siRNA molecule wherein the siRNA molecule comprises a polynucleotide having a sequence of 5′CAGCUCAGACUAUUACAAU 3′ (SEQ ID NO:3), or the complement thereof. In another embodiment, the agent is a siRNA molecule wherein the siRNA molecule comprises a polynucleotide having a sequence of 5′ CUUGGACUAUCUACAUUCCGGAAAG 3′ (SEQ ID NO:4), or the complement thereof. In still another embodiment, the agent is a siRNA molecule wherein the siRNA molecule comprises a polynucleotide having a sequence of 5′CUUUCCGGAAUGUAGAUAGUCCAAG 3′ (SEQ ID NO:5), or the complement thereof. In still another embodiment, the agent is a siRNA molecule wherein the siRNA molecule comprises a polynucleotide having a sequence of 5′ GAUGAAGAAUUUACAGCUCAGACUA 3′ (SEQ ID NO:6), or the complement thereof. In still another embodiment, the agent is a siRNA molecule wherein the siRNA molecule comprises a polynucleotide having a sequence of 5′ UAGUCUGAGCUGUAAAUUCUUCAUC 3′ (SEQ ID NO:7), or the complement thereof. In still another embodiment, the agent is a siRNA molecule wherein the siRNA molecule comprises a polynucleotide having a sequence of 5′ AAUUUACAGCUCAGACUAUUACAAU 3′ (SEQ ID NO:8), or the complement thereof. In still another embodiment, the agent is a siRNA molecule wherein the siRNA molecule comprises a polynucleotide having a sequence of 5′ AUUGUAAUAGUCUGAGCUGUAAAUU 3′ (SEQ ID NO:9), or the complement thereof. In a preferred embodiment, the agent contacts a cell using any of the well known procedures for introducing foreign nucleotide sequences into host cells. These include but are not limited to a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, a nanodendrimer, a virus, calcium phosphate nucleotide mediated nucleotide delivery, poly (D-arginine), electroporation, and microinjection. The use of nanoliposome, a nanoparticulate, a nanodendrimer for delivery of agents to a cell are demonstrated in FIGS. 5-11 and further described in application Ser. No. 10/835,520, filed on Apr. 26, 2004, herein incorporated by reference.

### Antisense Polynucleotides

In another embodiment, the agent is an antisense polynucleotide.

Specifically contemplated embodiments relate to the downregulation of Akt3 activity by the use of antisense polynucleotides, i.e., a nucleic acid complementary to, and which can preferably hybridize specifically to a coding mRNA nucleic acid sequence, e.g., Akt3 mRNA or a subsequence thereof. Binding of the antisense nucleotide to the Akt3 mRNA reduces the translation and/or stability of the Akt3 or B-Raf mRNA.

In the context of the invention, antisense polynucleotides can comprise naturally-occurring nucleotides, or synthetic species formed from naturally-occurring subunits or their close homologs. Antisense polynucleotides can also have altered sugar moieties or inter-sugar linkages. Exemplar), among these are the phosphorothioate and other sulfur containing species which are well known for use in the art. All such analogs are comprehended by this invention so long as they function effectively to hybridize Akt3 or B-Raf mRNA. For a general review see, e.g., Jack Cohen, Oligodeoxynucleotides, Antisense Inhibitors of Gene Expression, CRC Press, 1989; and Synthesis 1:1-5 (1988).

### Antagonists

The present also contemplates an embodiment where the agent that reduces Akt3 activity is an antisense polynucleotide. The invention also pertains to variants of the Akt3 proteins that function as Akt3 antagonists. Variants of the Akt3 protein can be generated by mutagenesis (e.g., discrete point mutation or truncation of the Akt3 protein). An antagonist of the Akt3 protein can inhibit one or more of the activities of the naturally occurring form of the Akt3 protein by, for example, competitively binding to a downstream or upstream member of a cellular signaling cascade which includes the Akt3 protein. Thus, specific biological effects can be elicited by treatment with a variant of limited function. The present invention contemplates treatment of a subject with a variant having a subset of the biological activities of the naturally occurring form of the protein has fewer side effects in a subject relative to treatment with the naturally occurring form of the Akt3 proteins.

Variants of the Akt3 protein that function as Akt3 antagonists can be identified by screening combinatorial libraries of mutants (e.g., truncation mutants) of the Akt3 proteins for Akt3 antagonist activity. The present invention contemplates a variegated library of Akt3 variants is generated by combinatorial mutagenesis at the nucleic acid level and is encoded by a variegated gene library. A variegated library of Akt3 variants can be produced by, for example, enzymatically ligating a mixture of synthetic oligonucleotides into gene sequences such that a degenerate set of potential Akt3 sequences is expressible as individual polypeptides, or alternatively, as a set of larger fusion proteins (e.g., for phage display) containing the set of Akt3 sequences therein. There are a variety of methods which can be used to produce libraries of potential Akt3 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential Akt3 sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

### Ribozymes

In yet another embodiment, the agent is a ribozyme. A ribozyme can be used to target and inhibit transcription of Akt3. A ribozyme is an RNA molecule that catalytically cleaves other RNA molecules. Different kinds of ribozymes have been described, including group I ribozymes, hammerhead ribozymes, hairpin ribozymes, RNAase P, and axhead ribozymes (see, e.g., Castanotto et al. 1994, Adv. In Pharmacology 25:289-317 for a general review of the properties of ribozymes).

The general features of hairpin ribozymes are described, e.g., in Hampel et al., 1990, Nucl. Acids Res., 18:299-304; Hampel et al., 1990, European Patent Publication No. 0 360 257; U.S. Pat. No. 5,254,678. Methods of preparing are well known to those of skill in the art (see, e.g., Wong-Staal et al., WO 94/26877; Ojwang et al., 1993, Proc. Natl. Acad. Sci. USA, 90:6340-6344; Yamada et al., 1994, Human Gene Therapy 1:39-45; Leavitt et al., 1995, Proc. Natl. Acad. Sci. USA, 92:699-703; Leavitt et al., 1994, Human Gene Therapy 5:1151-120; and Yamada et al., 1994, Virology 205:121-126).

### Inhibitors of Akt3 Polypeptide Activity

In yet another embodiment, Akt3 activity is decreased by agent that is an inhibitor of the Akt3 polypeptide. This can be accomplished in any of a number of ways, including by providing a dominant negative Akt3 polypeptide, e.g., a form of Akt3 that itself has no activity and which, when present in the same cell as a functional Akt3, reduces or eliminates the Akt3 activity of the functional Akt3. Design of dominant negative forms is well known to those of skill and is described, e.g., in Herskowitz, 1987, Nature, 329:219-22. Also, inactive polypeptide variants (muteins) can be used, e.g., by screening for the ability to inhibit Akt3 activity. Methods of making muteins are well known to those of skill (see, e.g., U.S. Pat. Nos. 5,486,463, 5,422,260, 5,116,943, 4,752,585, 4,518,504). In addition, any small molecule, e.g., any peptide, amino acid, nucleotide, lipid, carbohydrate, or any other organic or inorganic molecule can be screened for the ability to bind to or inhibit Akt3 activity.

### Peptides

In yet another embodiment, the agent, a peptide corresponding to the contiguous amino acid sequences of the pleckstrin homology domain, or the catalytic or the regulatory domain of Akt3, will decrease Akt 3 activity. With out wishing to be bound by this theory, the peptide is contemplated to act as a pseudosubstrate or a competitive inhibitor, thereby inhibiting Akt3 activity. In another embodiment, the peptide acts as a pseudosubstrate for the Akt3 catalytic or regulatory (tail) domain. In yet another embodiment, the peptide acts as a competitive inhibitor for the catalytic domain of Akt3. The inventors also contemplate that the peptide acts as a competitive inhibitor for the pleckstrin homology domain of Akt3. In yet another embodiment, peptide acts as a competitive inhibitor for the regulatory domain of Akt3. One of skill in the art can readily design and determine whether a peptide decreases the activity of Akt3. Obata T et al. J Biol. Chem. 275(46):36108-15 (2000), Niv M Y et al. J Biol. Chem. 279(2):1242-55. Epub 2003 (2004), Luo Y et al. Biochemistry. 43(5):1254-63 (2004).

For example, cells are incubated with the peptide under conditions suitable for assessing activity of Akt3. The activity of the Akt3 is assessed and compared with a suitable control, e.g., the activity of the same cells incubated under the same conditions in the absence of the peptide or a scrambled peptide, using Western blot analysis with an antibody recognizing threonine 305 or serine 472. Antibodies recognizing Akt3 are available from a number of sources, including Stratagene (La Jolla, Calif.) and IGeneX, Inc. (Palo Alto) to name a few. Alternatively, Akt3 activity could be assessed by mmunoprecipitating Akt3 and using the immunoprecipitate in an in vitro kinase assay in which Crosstide, a synthetic peptide substrate for Akt3 available from Discover Rx Corporation, Fremont, Calif., is phosphorylated by Akt3 to estimate activity. A greater or lesser activity of phosphorylation compared with the control indicates that the test peptide decreases the activity of said Akt3.

A peptide comprises about 5 to 30 amino acid residues in length, preferably between 10 and 20 amino acids in length. Peptide sequences of the present invention may be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these references are incorporated herein by reference.

### Small Molecules

The present also contemplates an embodiment where the agent that reduces Akt3 activity is a small molecule. Small molecules can also be used to regulate, for example, the function of the disclosed kinase, kinase receptors, molecules that interact with kinase receptors, and molecules in the signaling pathways of the kinase receptors. Those of skill in the art understand how to generate small molecules of this type, and exemplary libraries and methods for isolating small molecule regulators. The “small molecules”, as used herein preferably binds to Akt3 and/or B-Raf and inhibits at least one of its functions.

### Modulators and Binding Compounds

The compounds tested as modulators of an Akt3 and/or B-Raf protein can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

This invention contemplates high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, 1991, Int. J. Pept. Prot. Res. 37:487-493 and Houghton et al., 1991, Nature 354:84-88). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., 1993, Proc. Nat. Acad. Sci. USA 90:6909-6913), vinylogous polypeptides (Hagihara et al., 1992, J. Amer. Chem. Soc. 114:6568), nonpeptidal peptidomimetics with glucose scaffolding (Hirschmann et al., 1992, J. Amer. Chem. Soc. 114:9217-9218), analogous organic syntheses of small compound libraries (Chen et al., 1994, J. Amer. Chem. Soc. 116:2661), oligocarbamates (Cho et al., 1993, Science 261:1303), and/or peptidyl phosphonates (Campbell et al., 1994, J. Org. Chem. 59:658), nucleic acid libraries (see Ausubel, Berger and Sambrook, all supra), peptide nucleic acid libraries (see, e.g., U.S. Pat. No. 5,539,083), antibody libraries (see, e.g., Vaughn et al., 1996, Nature Biotechnology, 14:309-314 and PCT/US96/10287), carbohydrate libraries (see, e.g., Liang et al., 1996, Science, 274:1520-1522 and U.S. Pat. No. 5,593,853), small organic molecule libraries (see, e.g., benzodiazepines, Baum, 1993, C&EN, January 18, page 33; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### Chemotherapeutic Agents

In another embodiment, apoptosis is induced by decreasing Akt3 activity in conjunction with chemotherapeutic agents. As used herein, chemotherapy includes treatment with a single chemotherapeutic agent or with a combination of agents. Chemotherapeutic agents that may be used with the invention include, but are not limited to, alkylating agents, antimetabolites, antibiotics, natural or plant derived products, hormones and steroids (including synthetic analogs), and platinum drugs as described in Soengas M S, Lowe S W. Apoptosis and Melanoma Chemoresistance. Oncogene. 2003 May 19; 22(20):3138-51. Examples of agents within these classes are given below. Alkylating agents include, but are not limited to, for example nitrosoureas, nitrogen mustard, and triazenes. Nitrosoureas include, but are not limited to, for example carmustine, lomustine, and semustine. Nitrogen mustard, include but are not limited to, for example cyclophosphomide. Triazenes, include, but are not limited to, for example dacarbazine, and temozolomide. The FDA has approved dacarbazine for use in the treatment of melanoma. Antimetabolites include but are not limited to folic acid antagonists, pyrimidine analogs, purine analogs and adenosine deaminase inhibitors: methotrexate, 5-fluorouracil, floxuridine, cytarabine, 6-mercaptopurine, 6-thioguanine, fludarabine phosphate, pentostatine, and gemcitabine. Antibiotics that may be used with the present invention, include, but not limited to, for example anthracyclines. Examples of anthracyclines, include but are not limited to doxorubicin (adriamycin). Natural or plant derived products that may be used with the present invention, include, but are not limited to, for example vinca alkaloids, epipodophyllotoxins, taxanes. Examples of vinca alkaloids include but are not limited to for example, vincristine, and vinblastine. Examples of epipodophyllotoxins include but are not limited to for example etopside. Taxanes, include but are not limited to for example, taxol, paclitaxel, and docetaxel. Hormonal analogs and steroids that may be used with the present invention, include, but are not limited to, for example, antiestrogen, 17.alpha.-Ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, zoladex. Platinum drugs that may be used with the present invention, include, but are not limited to, for example, cisplatin, carboplatin, hydroxyurea, amsacrine, procarbazine, mitotane, mitoxantrone, levamisole, and hexamethylmelamine.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

### Irradiation

Irradiation can optionally be added to treatment regimens of the present invention. The term “irradiation” as used herein, has its conventional meaning and is only limited to the extent that the X-irradiation have sufficient energy to penetrate the body and be capable of inducing the release of tumor-specific antigens in vivo. The optimal radiation intensity for damaging a particular type of tumor is known to one of ordinary skill in the art.

### Apoptosis

One of skill in the art would know how to detect and/or measure apoptosis using a variety of methods, e.g., using the propidium iodide flow cytometry assay described in Dengler et al., (1995) Anticancer Drugs. 6:522-32, or by the in situ terminal deoxynucleotidyl transferase and nick translation assay (TUNEL analysis) described in Gorczyca, (1993) Cancer Res 53:1945-51.

### Treating a Melanoma Tumor

The present invention is based in part on the Inventors' observations showing that Akt3 regulates apoptosis and ^{V599E}B-Raf regulates growth and vascular development. This is a significant discovery since it identifies for the first time an effective combined targeted therapeutic for melanoma. As discussed infra, reducing Akt3 activity will increase the sensitivity of melanoma cells to apoptosis; therefore, agents that act through apoptosis such as conventional chemotherapeutics are more effective when Akt3 activity is reduced in melanoma cells. In one embodiment, the present invention provides a method for treating a melanoma tumor in a mammal comprising: administering to a tumor in a mammal an effective amount of an agent that reduces V599E B-Raf activity; and administering to a tumor in a mammal an effective amount of an agent that reduces Akt3 activity, thereby reducing the size of a tumor.

In a preferred embodiment, the agent for reducing Akt3 activity is a siRNA molecule. In an another embodiment, the agent is a siRNA molecule wherein the siRNA molecule that reduces Akt 3 activity comprises a polynucleotide having a sequence of 5′GGUCUAGCUACAGAGAAAUCUCGAU 3 (SEQ ID NO:10)′, 5′ CUAUCUACAUUCCGGAAAG 3′ (SEQ ID NO:1), 5′ GAAUUUACAGCUCAGACUA 3′ (SEQ ID NO:2), 5′ CAGCUCAGACUAUUACAAU 3′(SEQ ID NO:3), 5′CUUGGACUAUCUACAUUCCGGAAAG 3′ (SEQ ID NO:4), 5′CUUUCCGGAAUGUAGAUAGUCCAAG 3′ (SEQ ID NO:5), 5′GAUGAAGAAUUUACAGCUCAGACUA 3′ (SEQ ID NO:6), 5′UAGUCUGAGCUGUAAAUUCUUCAUC 3′ (SEQ ID NO:7), 5′AAUUUACAGCUCAGACUAUUACAAU 3′ (SEQ ID NO:8), 5′AUUGUAAUAGUCUGAGCUGUAAAUU 3′ (SEQ ID NO:9), or the complements thereof.

In one embodiment, the agent for reducing B-Raf activity is a siRNA molecule. In a preferred embodiment, the agent is a siRNA molecule wherein the siRNA molecule that reduces B-Raf activity comprises a polynucleotide having a sequence of 5′GGUCUAGCUACAGAGAAAUCUCGAU 3′ (SEQ ID NO:10), and/or 5′ GGACAAAGAAUUGGAUCUGGAUCAU 3′ (SEQ ID NO:11).

In a preferred embodiment, the agent that reduces Akt3 contacts a cell using any of the well known procedures for introducing foreign nucleotide sequences into host cells. These include a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, a nanodendrimer, a virus, calcium phosphate mediated nucleotide delivery, poly(D-arginine), electroporation, and microinjection. The use of a nanoliposome, a nanoparticulate, a nanodendrimer for delivery of agents to a cell are demonstrated in FIGS. 5-11 and further described in patent application Ser. No. 10/835,520, filed on Apr. 26, 2004, herein incorporated by reference.

In a preferred embodiment, the agent that reduces B-Raf activity contacts a cell using any of the well known procedures for introducing foreign nucleotide sequences into host cells. These include a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, a nanodendrimer, a virus, calcium phosphate mediated nucleotide delivery, poly(D-arginine), electroporation, and microinjection. The use of a nanoliposome, a nanoparticulate, a nanodendrimer for delivery of agents to a cell are demonstrated in FIGS. 5-11 and further described in patent application Ser. No. 10/835,520, filed on Apr. 26, 2004, herein incorporated by reference.

In a preferred embodiment, the present invention provides a method for the use of nano technology as the strategy to administer multiple agents to inhibit melanoma tumor development and increase or induce apoptosis. Combinations of Akt3 peptide; Akt 3 siRNA, V599E B-Raf siRNA, Paclitaxel, Carboplatin, Carmustine, Dacarbazine, or Vinblastine are simultaneously loaded into non-toxic liposomes. These liposomes effectively deliver this cargo into melanoma cells growing in culture. This is the first demonstration of simultaneous delivery of different therapeutics into cancer cells using a single delivery agent. Liposomes carrying combination therapeutic agents would travel in the bloodstream and enter the tumor vasculature to be taken up by exposed melanoma cells. This results in targeted killing of melanoma cells in tumors leading to regression of the tumor. The clinical utility of this approach is for delivering combination therapeutics into tumors. Covalently linking anti-CD63 antibody to the pegalation segment extending from the liposome will leads to preferential uptake by melanoma cells. Thus, stromal tissue takes up little or none of the liposome demonstrating targeted delivery of the liposome. The immuno liposome can enhance uptake into melanoma tumor cells versus control stromal tissue.

In another embodiment, the agent that reduces Akt 3 activity is an antisense polynucleotide. In one embodiment, the agent that reduces B-Raf activity is an antisense polynucleotide.

In yet another embodiment, the agent that reduces Akt 3 activity is a ribozyme. In still another embodiment, the agent that reduces B-Raf activity is a ribozyme. Ribozymes can be used to target and inhibit transcription of Akt3, B-Raf or both.

In yet another embodiment, Akt3 activity is decreased by agent that is an inhibitor of the Akt3 polypeptide. This can be accomplished in any of a number of ways, including by providing a dominant negative Akt3 polypeptide, e.g., a form of Akt3 that itself has no activity and which, when present in the same cell as a functional Akt3, reduces or eliminates the Akt3 activity of the functional Akt3. In yet another embodiment, B-Raf activity is decreased by agent that is an inhibitor of the B-Raf polypeptide. In a preferred embodiment, the B-Raf inhibitor is BAY 43-9006. Inhibitors of B-Raf include but are not limited to BAY 43-9006, commercially available from BAYER) or other commercially available B-Raf inhibitors. Inhibitors of B-Raf may additionally include competitive and noncompetitive B-Raf inhibitors. A competitive B-Raf inhibitor is a molecule that binds the B-Raf enzyme in a manner that is mutually exclusive of substrate binding. Typically, a competitive inhibitor of B-Raf will bind to the active site. A noncompetive B-Raf inhibitor can be one which inhibits the synthesis of B-Raf, but its binding to the enzyme is not mutually exclusive over substrate binding. B-Raf inhibitors contemplated by this invention are compounds that reduce the activity of B-Raf in animal cells without any significant effect on other cellular activities, at least at comparable concentrations. However, this inhibition can be accomplished in any of a number of ways, including by providing a dominant negative B-Raf polypeptide, e.g., a form of B-Raf that itself has no activity and which, when present in the same cell as a functional B-Raf, reduces or eliminates the B-Raf activity of the functional B-Raf.

In yet another embodiment, the agent that reduces Akt 3 activity is a peptide corresponding to the contiguous amino acid sequences of the pleckstrin homology domain, or the catalytic or the regulatory domain of Akt3.

The present also contemplates an embodiment where the agent that reduces Akt3 activity is a small molecule. In another embodiment, the present also contemplates embodiments where the agent that reduces B-Raf activity is a small molecule.

In another embodiment, the method for treating a melanoma tumor includes administering chemotherapeutic agents. As used herein, chemotherapy includes treatment with a single chemotherapeutic agent or with a combination of agents. Chemotherapeutic agents that may be used with the invention include, but are not limited to, alkylating agents, antimetabolites, antibiotics, natural or plant derived products, hormones and steroids (including synthetic analogs), and platinum drugs as described

In another embodiment, the method for treating a melanoma tumor in a mammal includes irradiation therapy.

In preferred embodiments, the methods of the present invention can be used to treat melanomas by having a significant effect on cell death (e.g. by apoptosis) as well as proliferation and angiogenesis. One of skill in the art would be familiar with methods measuring the size of a tumor to measure, for example, the regression or reduction in tumor size, angiogenesis, and apoptosis. Advantageously, chemotherapeutic agents can be administered in relatively low doses (and/or less frequently) to minimize potential toxic side effects against normal, untransformed cells.

Thus, the present invention also provides methods of inducing a significant level of cancer cell death (e.g., apoptosis) and inhibition of melanoma tumor development in a subject with a melanoma, comprising administering, concurrently or sequentially, effective amounts of an agent that reduces Akt3 activity and an agent that reduces B-Raf activity. As used herein, “concurrently” refers to simultaneously in time, or at different times during the course of a common treatment schedule; and “sequentially” administering one of the agents of the method for reducing Akt3 or B-Raf activity, and an additional agent for reducing B-Raf or Akt3 activity wherein the second agent can be administered substantially immediately after the first agent, or the second agent can be administered after an effective time period after the first agent; the effective time period is the amount of time given for realization of maximum benefit from the administration of the first agent.

Targeting Akt3 together with mutant ^{V599E}B-Raf and selected chemotherapeutics has a synergistic, more potent and prolonged effect than targeting either alone. This provides a rational basis for combining targeted therapies together with selected chemotherapeutics, which does not currently exist for melanoma.

Uses of Akt3 and/or B-Raf Protein and Akt3-Related and/or B-Raf-Related Proteins

The proteins of the invention have a number of different specific uses. Both Akt3 and B-Raf are key proteins contributing to melanoma development. Akt3 and/or B-Raf protein and Akt3 or B-Raf related protein are used in methods that assess the status of Akt3 and/or B-Raf gene products in normal versus cancerous tissues, thereby elucidating the malignant phenotype. Typically, polypeptides from specific regions of an Akt3 or B-Raf protein may be used to assess the presence of perturbations (such as deletions, insertions, point mutations etc.) in those regions (such as regions containing one or more motifs). A non-limiting example includes use of antibodies targeting Akt3 and/or B-Raf protein and Akt3-related and/or B-Raf-related protein comprising the amino acid residues of one or more of the biological motifs contained within an Akt3 and/or B-Raf polypeptide sequences respectively in order to evaluate the characteristics of this region in normal versus cancerous tissues or to elicit an immune response to the epitope. Alternatively, Akt3-related and/or B-Raf-related proteins that contain the amino acid residues of one or more of the biological motifs in an Akt3 and/or B-Raf protein respectively are used to screen for factors that interact with that region of Akt3 and/or B-Raf.

Both Akt3 and B-Raf protein fragments/subsequences are particularly useful in generating and characterizing domain-specific antibodies (e.g., antibodies recognizing an extracellular or intracellular epitope of an Akt3 or B-Raf protein), for identifying agents or cellular factors that bind to Akt3 or B-Raf or a particular structural domain thereof, and in various therapeutic and diagnostic contexts, including but not limited to diagnostic assays, cancer vaccines and methods of preparing such vaccines.

The protein encoded by the Akt3 and/or B-Raf gene, or by analogs, homologs or fragments thereof, has a variety of uses, including but not limited to generating antibodies and in methods for identifying ligands and other agents and cellular constituents that bind to an Akt3 and/or B-Raf gene product. Antibodies raised against an Akt3 or B-Raf protein or fragment thereof are useful in diagnostic and prognostic assays, and imaging methodologies in the management of human melanoma cancer characterized by expression of Akt3 or B-Raf protein.

Various immunological assays useful for the detection of Akt3 and/or B-Raf protein may be used, including but not limited to various types of radioimmunoassays, enzyme-linked immunosorbent assays (ELISA), enzyme-linked immunofluorescent assays (ELIFA), immunocytochemical methods, and the like. Antibodies can be labeled and used as immunological imaging reagents capable of detecting Akt3 or B-Raf-expressing cells.

Antibodies to Akt3 and/or B-Raf in Melanoma

According to the invention, the Akt3 and/or B-Raf polypeptide encoded by the Akt3 isoform or B-Raf isoform respectively found in melanomas includes fragments thereof, including fusion proteins, may be used as an antigen or immunogen to generate antibodies. Preferably, the antibodies specifically bind the human Akt3 isoform, but do not bind other forms of Akt. Preferably, the antibodies specifically bind the human B-Raf isoform, but do not bind other forms of B-Raf.

A molecule is “antigenic” when it is capable of specifically interacting with an antigen recognition molecule of the immune system, such as an immunoglobulin (antibody) or T cell antigen receptor. An antigenic polypeptide or peptide contains at least about 5, and preferably at least about 10, amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

Such antibodies include but are not limited to polyclonal, monoclonal, chimeric, single chain, Fab fragments, and a Fab expression library. The anti-Akt3 antibodies of the invention may be cross reactive, e.g., they may recognize Akt3 from different species. Similarly, the anti-B-Raf antibodies of the invention may be cross reactive, e.g., they may recognize B-Raf from different species. Polyclonal antibodies have greater likelihood of cross reactivity. Alternatively, an antibody of the invention may be specific for a single form of Akt3 or B-Raf. Preferably, such an antibody is specific for human melanoma Akt3 or B-Raf.

Various procedures known in the art may be used for the production of polyclonal antibodies. For the production of antibody, various host animals can be immunized by injection with the Akt3 or B-Raf polypeptide, or a derivative (e.g., fragment or fusion protein) thereof, including but not limited to rabbits, mice, rats, sheep, goats, etc. In one embodiment, the Akt3 or B-Raf polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the Akt3 or B-Raf polypeptide, or fragment, analog, or derivative thereof, any technique that provides for the production of antibody molecules by continuous cell lines in culture may be used. These include but are not limited to the hybridoma technique originally developed by Kohler and Milstein [Nature 256:495-497 (1975)], as well as the trioma technique, the human B-cell hybridoma technique [Kozbor et al., Immunology Today 4:72 1983); Cote et al., Proc. Natl. Acad. Sci. U.S.A. 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. In an additional embodiment of the invention, monoclonal antibodies can be produced in germ-free animals [International Patent Publication No. WO 89/12690, published Dec. 28, 1989]. In fact, according to the invention, techniques developed for the production of “chimeric antibodies” [Morrison et al., J. Bacteriol. 159:870 (1984); Neuberger et al., Nature 312:604-608 (1984); Takeda et al., Nature 314:452-454 (1985)] by splicing the genes from a mouse antibody molecule specific for an Akt3 polypeptide together with genes from a human antibody molecule of appropriate biological activity can be used; such antibodies are within the scope of this invention. Such human or humanized chimeric antibodies are preferred for use in therapy of human diseases (described infra), since the human or humanized antibodies are much less likely than xenogenic antibodies to induce an immune response, in particular an allergic response, themselves.

Techniques described for the production of single chain Fv (scFv) antibodies [U.S. Pat. Nos. 5,476,786 and 5,132,405 to Huston; U.S. Pat. No. 4,946,778] can be adapted to produce Akt3 polypeptide-specific single chain antibodies. An additional embodiment of the invention utilizes the techniques described for the construction of Fab expression libraries [Huse et al., Science 246:1275-1281 (1989)] to allow rapid and easy identification of monoclonal Fab fragments with the desired specificity for an Akt3 polypeptide, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of an Akt3 or B-Raf polypeptide or peptide, one may assay generated hybridomas for a product which binds to an Akt3 or B-Raf polypeptide fragment containing such epitope. For selection of an antibody specific to an Akt3 or B-Raf polypeptide or peptide from a particular species of animal, one can select on the basis of positive binding with Akt3 or B-Raf polypeptide or peptide expressed by or isolated from cells of that species of animal.

Identification of Molecules that Interact with Akt3 or B-Raf

The protein and nucleic acid sequences for Akt3 and B-Raf found in melanoma allow a skilled artisan to identify proteins, small molecules and other agents that interact with Akt3 or B-Raf, as well as pathways activated by Akt3 or B-Raf via any one of a variety, of art accepted protocols. For example, one can utilize one of the so-called interaction trap systems (also referred to as the “two-hybrid assay”). In such systems, molecules interact and reconstitute a transcription factor which directs expression of a reporter gene, whereupon the expression of the reporter gene is assayed. Other systems identify protein-protein interactions in vivo through reconstitution of a eukaryotic transcriptional activator, see, e.g., U.S. Pat. No. 5,955,280 issued Sep. 21, 1999, U.S. Pat. No. 5,925,523 issued Jul. 20, 1999, U.S. Pat. No. 5,846,722 issued Dec. 8, 1998 and U.S. Pat. No. 6,004,746 issued Dec. 21, 1999. Algorithms are also available in the art for genome-based predictions of protein function (see, e.g., Marcotte, et al., Nature 402: 4 Nov. 1999, 83-86).

Alternatively one can screen peptide libraries to identify molecules that interact with Akt3 or B-Raf protein sequences. In such methods, peptides that bind to Akt3 or B-Raf are identified by screening libraries that encode a random or controlled collection of amino acids. Peptides encoded by the libraries are expressed as fusion proteins of bacteriophage coat proteins, the bacteriophage particles are then screened against the Akt3 or B-Raf protein(s) respectively.

Accordingly, peptides having a wide variety of uses, such as therapeutic, prognostic or diagnostic reagents, are thus identified without any prior information on the structure of the expected ligand or receptor molecule.

### Pharmaceutical Composition

In one embodiment, a pharmaceutical composition for treating a melanoma tumor comprises an agent that reduces Akt3 activity; and

a carrier is provided. Carriers suitable for use with the present invention will be known to those of skill in the art. Such carriers include but are not limited to a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, and calcium phosphate nucleotide-mediated nucleotide delivery.

In another embodiment, the pharmaceutical composition comprises an agent that includes but is not limited to a siRNA molecule, an antisense molecule, an antagonist, a ribozyme, an inhibitor, a peptide, and a small molecule. In other embodiments, the small interfering RNA (siRNA) molecules includes the polynucleotides 5′ GGUCUAGCUACAGAGAAAUCUCGAU 3′ (SEQ ID NO:10), 5′ CUAUCUACAUUCCGGAAAG 3′ (SEQ ID NO:1), 5′ GAAUUUACAGCUCAGACUA 3′ (SEQ ID NO:2), 5′ CAGCUCAGACUAUUACAAU 3′ (SEQ ID NO:3), 5′CUUGGACUAUCUACAUUCCGGAAAG 3′ (SEQ ID NO:4), 5′CUUUCCGGAAUGUAGAUAGUCCAAG 3′ (SEQ ID NO:5), 5′GAUGAAGAAUUUACAGCUCAGACUA 3′ (SEQ ID NO:6), 5′UAGUCUGAGCUGUAAAUUCUUCAUC 3′ (SEQ ID NO:7), 5′AAUUUACAGCUCAGACUAUUACAAU 3′ (SEQ ID NO:8), 5′AUUGUAAUAGUCUGAGCUGUAAAUU 3′ (SEQ ID NO:9) or the complements thereof. In yet another embodiment, the pharmaceutical composition comprises an agent that is a peptide that acts as a pseudosubstrate for Akt3. In another embodiment, the peptide acts as a pseudosubstrate for a catalytic domain of Akt3.

In still another embodiment, the agent that reduces Akt3 activity is a peptide that acts as a competitive inhibitor for Akt3. The inventors contemplate that the peptide can act as a competitive inhibitor for a catalytic domain of Akt3, a pleckstrin homology domain of Akt3, and/or for a regulatory domain of Akt3. In yet another embodiment, the pharmaceutical composition includes an agent that reduces B-Raf activity. The inventors contemplate that the agent includes a siRNA molecule, an antisense molecule, an antagonist, a ribozyme, an inhibitor, a peptide, and a small molecule. In another embodiment, the agent that reduces B-Raf activity is a small interfering RNA (siRNA) molecule comprises: a polynucleotide 5′ GGUCUAGCUACAGAGAAAUCUCGAU 3′ (SEQ ID NO:10), or the complement thereof or a polynucleotide 5′ GGACAAAGAAUUGGAUCUGGAUCAU 3′ (SEQ ID NO:11), or the complement thereof.

The following Examples are offered by way of illustration and not by way of limitation.

### EXAMPLES FOR AKT3

### Materials and Methods

### Example 1

### SiRNA Mediated Downregulation of Akt Isoforms

To demonstrate the specificity of siRNA against Akt1, Akt2 and Akt3 (Dharmacon) in UACC 903 cells, HA-tagged Akt1, Akt2 or Akt3 constructs were co-nucleofected together with each respective siRNA. The Akt constructs used for these studies have been described previously (Sun et al., Am J Path 159:431-437 (2001); Mitsuuchi et al., J Cellular Biochem 70:433-441 (1998); and Brodbeck et al., J Biol Chem 274:9133-9136 (1999)). Each construct (5 μg), either alone or in combination with 100 pmol or 200 pmol of each respective siRNA, was introduced into 7×10⁵ UACC 903 cells via nucleofection using an Amaxa Nucleofector. The resultant transfection efficiency using constructs expressing GFP was >60%. Protein lysates were harvested 72 h later and Western blot analysis performed as described previously (Stahl et al., Cancer Res 63:2891-2897 (2003)). Nucleofection with siRNA was also used to knockdown endogenous expression of the Akt isoforms and/or PTEN (Dharmacon) in melanocytes and in the melanoma cell lines UACC 903, SK-MEL-24, WM115, and WM35. The Amaxa nucleofection reagents and protocol for melanocytes was also used with WM35 cells while other cell lines were nucleofected using Amaxa Solution R/program K-17. The growth conditions for these cell lines have been described previously (Stahl et al., Cancer Res 63:2891-2897 (2003); Hsu et al., In Human Cell Culture, J.R.W.M.a.B. Palsson, editor. Great Britain: Kluwer Academic Publishers. 259-274 (1999)).

### Example 2

### Western Blotting, Immunoprecipitation and Kinase Assays

The Western blot procedure and antibodies used, except for Akt2 (Santa Cruz) and Akt3 (Upstate Biotech), have been reported previously (Stahl et al., Cancer Res 63:2891-2897 (2003)). For immunoprecipitation, protein was collected following addition of protein lysis buffer (50 mM Tris-HCl pH 7.5, 0.1% Triton X-100, 1 mM EDTA, 1 mM EGTA, 50 mM NaCl, 10 mM sodium β-glycerol phosphate, 5 mM sodium pyrophosphate, 1 mM sodium orthovanadate, 0.1% 2-mercaptoethanol, and 0.5% protease inhibitor cocktail (Sigma)) to plates of cells followed by snap freezing in liquid nitrogen. Cellular debris was pelleted by centrifugation (≧10,000×g) of lysates and protein concentration quantitated using the BioRad BCA Protein Assay. Protein for immunoprecipitation (100 μg) was incubated with 2 μg of Akt2 or 5 μl of Akt3 antibody overnight at 4° C. with constant mixing. Next 15 μl of equilibrated GammaBind G Sepharose beads (Amersham Biosciences) were added to each tube and incubated 2 h (4° C.) with constant mixing. Pelleted beads were washed twice with lysis buffer to remove unbound antibody and protein. Samples were then resuspended and electrophoresed under reducing conditions according to the protocol provided by Invitrogen Life Technologies with the NuPage Gel System. Western blots were probed with phosphor-Akt and quantitated by densitometry as described previously (Stahl et al., Cancer Res 63:2891-2897 (2003)).

For Akt kinase assay, 15 μl of equilibrated GammaBind G Sepharose beads were washed with 200 μl of lysis buffer, then incubated with 2 μg of Akt2 or 5 μl of Akt3 antibody in a volume of 400 μl at 4° C. with constant mixing for ≧2 h. Microcystin (1 μM) from MP Biomedicals was added to the lysis buffer to ensure complete inactivation of cellular PP1 and PP2 phosphatases. The antibody/sepharose complex was washed twice with 750 μl of lysis buffer, then incubated with 100 μg of protein in a volume of 400 μl for ≧1.5 h at 4° C. with constant mixing. This complex was washed with 500 μl lysis buffer (3×) then once with 500 μl of Assay Dilution Buffer (20 mM MOPS, pH 7.2, 25 mM μ-glycerol phosphate, 1 mM sodium orthovanadate, and 1 mM DTT). PKA Inhibitor peptide (10 μM) from Santa Cruz, 37.5 μM ATP, 17 mM MgCl₂, 0.25 μCi/μl γ-³²P-ATP, and 90 μM Akt specific substrate Crosstide from UIpstate Biotechnology were added to the tubes in assay dilution buffer and incubated at 35° C. for 10 m with continuous mixing. Next, 20 μl of liquid was transferred to phosphocellulose paper, which was washed 3× for 5 m with 40 ml of 0.75% phosphoric acid. Following a 5 m acetone wash, the phosphocellulose was allowed to dry, transferred to a scintillation vial with 5 ml of Amersham Biosciences scintillation fluid and cpm measured in a Beckman Coulter LS 3801 Liquid Scintillation System.

### Example 3

### Tumor Studies and Apoptosis Measurements

Collection of melanoma tumors from human patients was performed according to protocols approved by the Penn State Human Subjects Protection Office, the Dana-Farber Cancer Institute Protocol Administration Office, and Cooperative Human. Tissue Network. Formalin-fixed paraffin embedded archival melanoma specimens were used for immunohistochemistry to measure phosphorylated Akt. Sixty-three formalin-fixed paraffin embedded archival specimens of melanocytic lesions were used for immunohistochemistry experiments with the phosphor-Akt (Ser473) monoclonal antibody (Cell Signaling Technology) at a 1:50 titer according to the manufacturer's recommended protocol. Specificity and intensity of staining was determined through qualitative comparison to internal blood vessel endothelium, squamous epithelium or smooth muscle controls present in each specimen.

Tumor protein for Western blotting or immunoprecipitation was collected by using a mortar and pestle chilled in liquid nitrogen to pulverize tumor material flash frozen in liquid nitrogen, which consisted of >60% tumor material. One ml of protein lysis buffer was added for every 200 mg of tissue powder and sonicated for 2 m (15 s intervals) in an ice-filled sonicator bath. The samples were centrifuged (˜12,000×g) at 4° C. for 10 minutes. The supernatant was transferred to a clean tube and quantitated using the BioRad BCA Protein Assay.

Animal experimentation was performed according to protocols approved by the Institutional Animal Care and Use Committee at the Pennsylvania State College of Medicine. Athymic female nude mice were purchased from Harlan Sprague Dawley and tumor kinetics measured by s.c. injection of 1×10⁶ cells in 0.2 ml of DMEM containing 10% FBS above the left and right rib cages of 4-6 week old nude mice. For animal experimentation involving siRNA, 1×10⁶ UACC 903 cells were nucleofected with siRNA to Akt isoforms and 48 h later, nucleofected cells in 0.2 ml of DMEM containing 10% FBS were s.c. injected above the left and right rib cages of nude mice. The dimensions of the developing tumors were measured on alternating days using calipers. When measuring apoptosis, 5×10⁶ cells were injected per site and 4-6 tumors were harvested 4 days later. Apoptosis measurements were performed on formalin-fixed, paraffin-embedded tumor sections using the Roche TUNEL TMR Red Apoptosis kit as described previously (Stahl et al., Cancer Res 63:2891-2897 (2003)). A minimum of 8-fields were counted from three or four different tumor sections, and the number of TUNEL positive cells was expressed as the percentage of apoptotic cells.

### Example 4

### Statistics

For statistical analyses, the Student's t-test was used for pair wise comparisons and the One-way ANOVA or Kruskal Wallis ANOVA on Ranks used for group wise comparisons, followed by the appropriate post hoc tests (Dunnett's or Dunn's). Results were considered significant at a P-value of <0.05.

TABLE 1
Relative intensity of p-Akt staining in common nevi, dysplastic
nevi, primary melanomas and metastases from melanoma patients.

p-Akt Staining Intensity (%)

| Category (# of samples) | Moderate to weak¹ | Strong² |
|---|---|---|
| Common nevi (14) | 100 | 0^{a} |
| Dysplastic nevi (25) | 88 | 12^{b} |
| Primary melanomas (15) | 47 | 53^{c} |
| Metastatic melanomas (9) | 33 | 67^{d} |

¹Tumor cells were stained to an intensity similar to that in pericytes adjacent to blood vessels in the tumor section.
²Tumor cells were stained to an intensity greater than pericytes adjacent to blood vessels in the tumor section.
Statistics, P < 0.5 for ^{a} versus ^{c,d} and ^{b} versus ^{d}.

### Experimental Results

### Example 5

### Isoform Specific siRNA Identify Akt3 Involvement in Melanoma

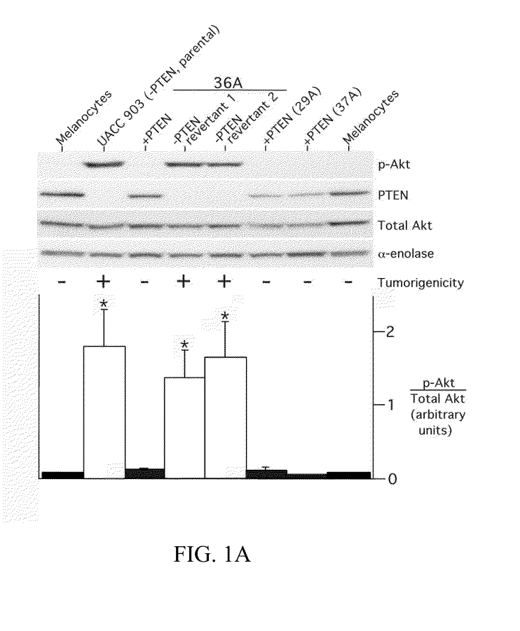

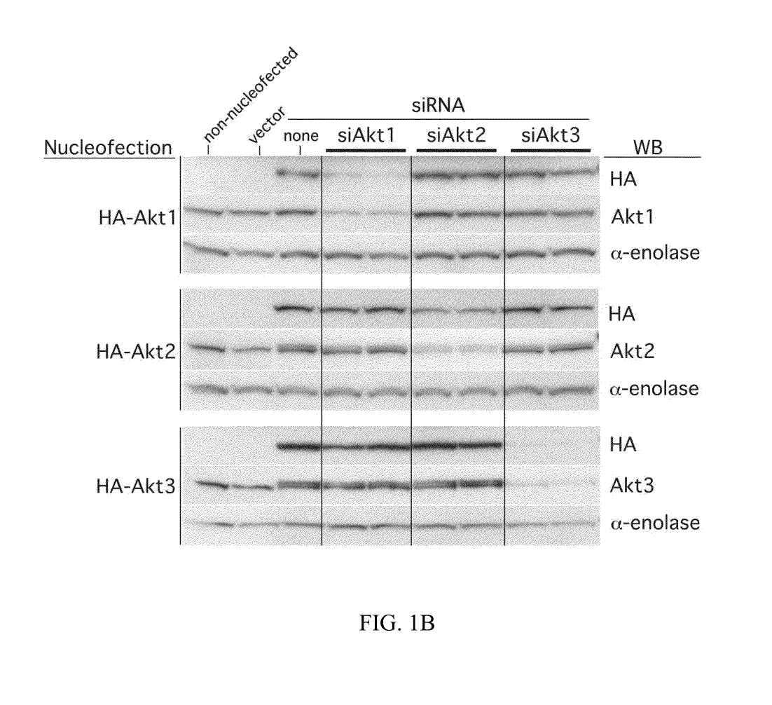

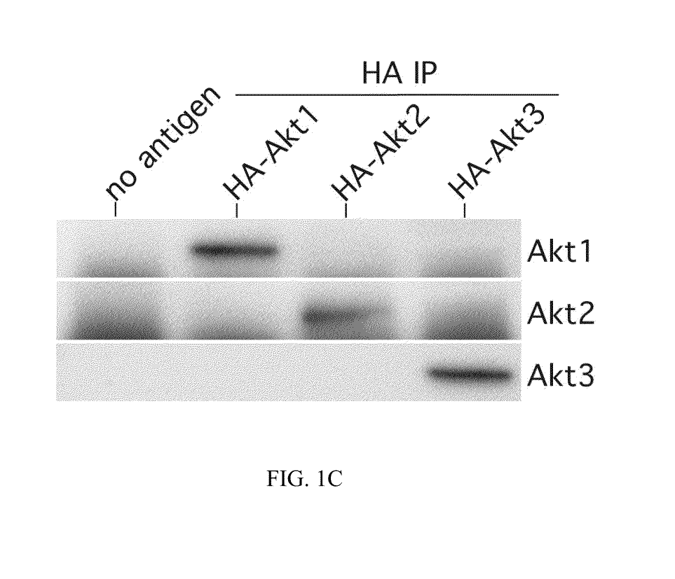

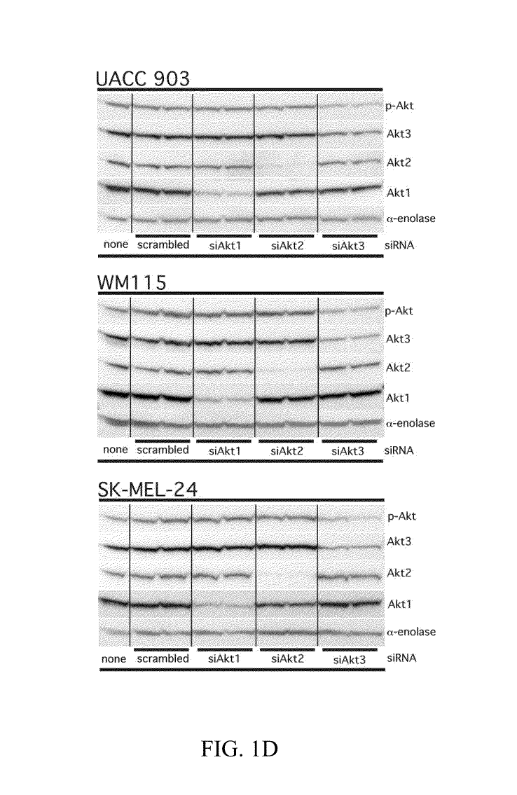

In a recently described experimental genetic melanoma model (Stahl et al., Cancer Res 63:2891-2897 (2003)), deregulated Akt activity (through PTEN loss) was demonstrated to play a critical role in melanoma tumorigenesis by decreasing the apoptotic capacity of melanoma cells (Stahl et al., Cancer Res 63:2891-2897 (2003)). We reasoned that since this model reflected the importance of Akt in melanoma tumorigenesis, it could be used to identify the specific Akt isoforms whose deregulated activity controls melanoma tumor development. Validating earlier results (Stahl et al., Cancer Res 63:2891-2897 (2003)), parental UACC 903 (-PTEN) cells had elevated total phosphorylated Akt expression (a measure of activity) (FIG. 1A). Expression of PTEN in UACC 903 cells resulted in diminished Akt activity in three independently derived cell lines (36A, 29A, and 37A), which was reversed in revertant cell lines that lost functional PTEN activity (36A revertants) during tumorigenesis.

To identify the predominant Akt isoform active in melanomas, we used siRNA specific to each Akt isoform to determine the extent to which each isoform reduced the amount of phosphorylated (active) Akt in the parental UACC 903 (-PTEN) cell line. The specificity of knocking down expression for each Akt isoform in UACC 903 cells was determined by co-nucleofecting constructs expressing tagged HA-Akt1, HA-Akt2 or HA-Akt3 together with siRNA specific for each isoform. Attesting to specificity, each Akt siRNA was found to only reduce expression of the Akt isoform against which it was made, shown in FIG. 1B. Next, siRNA to each Akt isoform was nucleofected into the UACC 903 cell line as well as two additional independently derived melanoma cells lines (WM115 and SK-MEL-24) to determine which siRNA lowered the level of phosphorylated (active) Akt in these cells (FIG. 1C). While siRNA to Akt1 or Akt2 had only a negligible, non-significant effect, siRNA to Akt3 significantly reduced the levels of phosphorylated total Akt, suggesting that Akt3 was the isoform regulating tumor development in the UACC 903 (PTEN) model (Stahl et al., Cancer Res 63:2891-2897 (2003)). Since all three independently derived melanoma cell lines indicated that Akt3 was the predominant active isoform in melanomas, subsequent experiments focused on examining Akt3 deregulation, and used Akt2 as a control for comparison since it has been reported to be amplified in multiple types of cancers (Chen et al., Proc Nat Acad Sciences USA 89:9267-9271 (1992); Cheng et al., Proc Nat Acad Sciences USA 93:3636-3641 (1996); Lu et al., Chung-0Hua I Hsuch Tsa Chili [Chinese Medical Journal] 75:679-682 (1995); Bellacosa et al., Int J Cancer 64:280-285 (1995); and van Dekken et al., Cancer Res 59:748-752 (1999)).

To further confirm that Akt3 was the predominant isoform whose activity was specifically reduced by PTEN in the UACC 903 (PTEN) tumorigenesis model, Akt3 activity was measured by immunoprecipitating total Akt3 or Akt2 from cell lysates followed by Western blotting to estimate the amount of phosphorylated (active) Akt in the immunoprecipitated (FIG. 1D). Phosphorylated Akt3, but not Akt2, levels were elevated in the parental UACC 903 cell line as well as the two tumorigenic revertant 36A cell lines that lack PTEN. In contrast, barely detectable levels of phosphorylated Akt3, were observed in the 36A, 29A or 37A cell lines, which had low levels of Akt activity, ostensibly due to PTEN expression (Stahl et al., Cancer Res 63:2891-2897 (2003)). To verify that the phosphorylated levels of Akt3 reflected active Akt, immunoprecipitated Akt3 and Akt2 were assayed in an in vitro kinase assay, and the results for Akt3 are shown in FIG. 1E. A statistically significant difference in Akt3 (FIG. 1E), but not Akt2 activity (data not shown), was identified in revertants (-PTEN) compared to PTEN expressing cells (P<0.05). Collectively, these results indicate that Akt3 activity was specifically regulated by PTEN in the UACC 903 (PTEN) tumorigenesis model.

### Example 6

### Increased Akt3 Activity Occurs Early During Melanoma Tumor Progression

Melanocytes are thought to be capable of transforming directly into a melanoma (Herlyn, M., Molecular and cellular biology of melanoma: Austin: R.G. Landes Co. (1993)). Alternatively, melanocytes can follow a model of tumor progression in which they evolve in a stepwise fashion from common nevi, to atypical nevi, to melanoma in situ (Radial and vertical growth phases), and finally to metastatic melanomas (Herlyn, M., Molecular and cellular biology of melanoma: Austin: R.G. Landes Co. (1993)). Regardless of the process, the evolution of more aggressive tumor cells requires the accumulation of alterations affecting tumor suppressor genes and oncogenes. These, in turn, result in sub-populations of cells that have ever-increasing selective growth or survival advantages that promote the tumorigenic process.

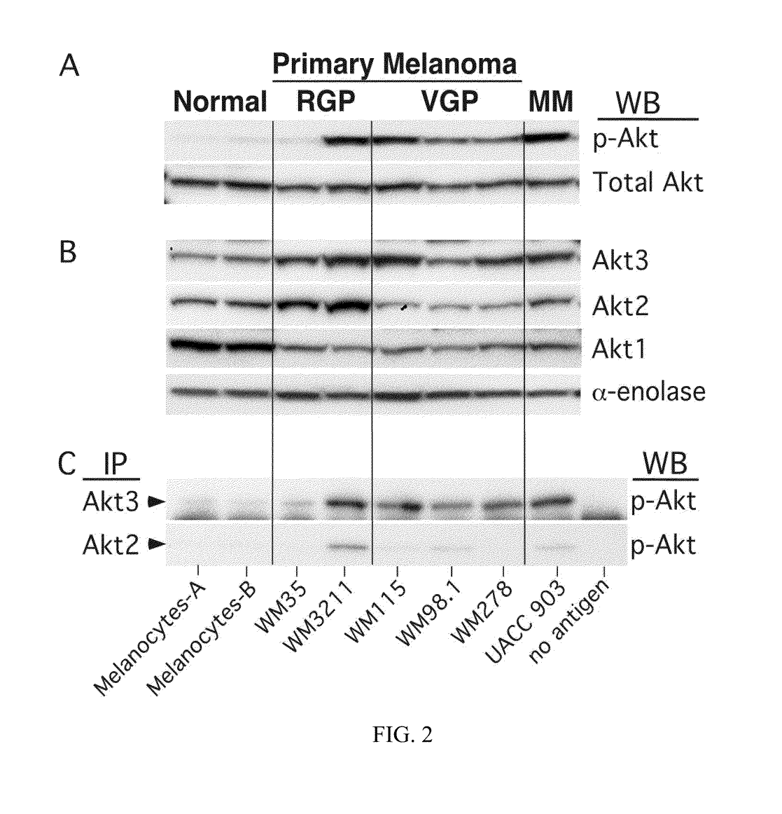

To provide evidence for the selective involvement of Akt3 during melanoma tumor progression, Akt3 and Akt2 expression and activity were measured in a melanoma tumor progression model (generously provided by Dr. Meenhard Herlyn) (Herlyn, M., Molecular and cellular biology of melanoma: Austin: R.G. Landes Co. (1993); Hsu et al., In Human Cell Culture J.R.W.M.a.B. Palsson, editor. Great Britain: Kluwer Academic Publishers. 259-274 (1999)). In this progression model, melanocytes are compared to low passage cell lines established from primary melanoma tumors at the radial (WM35 and WM3211) and vertical (WM115, WM98.1 and WM278) stages of growth. In comparison to melanocytes, FIG. 2A shows that one of the two radial growth phase and all three of the vertical growth phase cell lines had elevated phosphorylated Akt, which suggested that Akt activity increased early during primary melanoma development in the radial growth phase. Next, expression of the Akt3 isoform was examined by Western blotting and compared to Akt2 expression in these cell lines (FIG. 2B). Akt3 expression was found to be elevated in all except the WM98.1 radial growth phase cell line which compared to melanocytes. Since expression does not necessarily reflect activity, the amount of active Akt3 was examined by immunoprecipitation of Akt3 or Akt2 followed by Western blot analysis to measure the level of phosphorylated Akt in the immunoprecipitate. In comparison to melanocytes, Akt3 activity was elevated in all except the WM35 radial growth phase cell line (FIG. 2C). Note that even though Akt3 protein expression in the WM98.1 vertical growth phase cell line was similar to that observed in melanocytes, Akt3 activity was significantly higher. In contrast to the Akt3 results, Akt2 expression was elevated only in the radial growth phase cell lines in comparison to melanocytes (FIG. 2C). However, only the WM3211 radial growth phase cell line had a corresponding increase in Akt2 activity, but also had elevated Akt3 activity when compared to melanocytes. These data suggest that Atk3 was the predominantly involved Akt isoform active in the melanoma tumor progression model.

### Example 7

### Frequency of Akt3 Deregulation in Tumors from Melanoma Patients

Since the foregoing experiments identified Akt3 as the predominantly active Akt isoform in both the UACC 903 (PTEN) tumorigenesis and melanoma tumor progression models, subsequent in vivo studies focused on establishing the frequency of Akt3 deregulation in tumors from melanoma patients. The relative intensity of total phosphorylated Akt was initially assessed in melanocytic lesions by immunohistochemical analysis of common nevi, dysplastic nevi, primary melanomas and metastases from melanoma patients to determine the frequency of Akt activation (Table 1). While moderate levels of staining were detected in 100% of common nevi, strong staining was observed in 12% of dysplastic nevi, 53% of primary melanomas and 67% of metastatic melanomas. These results suggest that while Akt activity may serve some unidentified role in nevi development, deregulated Akt activity is indicative of a more important role in advanced stage melanomas.

Analysis of the genomic regions containing the Akt1, Akt2 and Akt3 genes, from a published report (Bastian et al., Cancer Res 58:2170-2175 (1998)), has not found amplification. However, the 1q43-44 region containing Akt3 does undergo copy number increases (Bastian et al., Cancer Res 58:2170-2175 (1998); Thompson et al., Cancer Genet Cytogenet 83:93-104 (1995); Mertens et al., Cancer Res 57:2765-2780 (1997)), which suggests overexpression as a mechanism contributing to increased Akt3 activity in melanomas. In contrast, the 14q32 region containing Akt1 and the 19q13 region containing Akt2 remain unchanged or tend to undergo loss (Bastian et al., Cancer Res 58:2170-2175 (1998); Thompson et al., Cancer Genet Cytogenet 83:93-104 (1995); Mertens et al., Cancer Res 57:2765-2780 (1997)). To establish whether increased Akt3 expression could be a selective mechanism leading to increased activity, protein lysates from melanoma patients' tumors were extracted to compare the level of expression and activity of Akt3 and Akt2. Protein was extracted from 31 metastatic melanomas and analyzed by Western blotting to determine the level of expression and activity of Akt3 and Akt2 in the tumor material.

Three independent Western blots were used to quantitate expression in each sample, which was then compared to expression in melanocytes (FIG. 2D). Overall, 61% (19/31) of the tumors had elevated Akt3 protein expression, ranging from a ˜2-9 fold increase over the expression observed in melanocytes compared to 10% (3/31) for Akt2. These results are consistent with type of copy number increases of the region of chromosome 1q43-44 containing the Akt3 gene reported in the literature to occur in melanoma tumors (Bastian et al. Cancer Res 58:2170-2175; Thompson et al., Cancer Genet Cytogenet 83:93-104 (1995); and Mertens et al., Cancer Res 57:2765-2780 (1997)). Approximately 55% (6/11) of primary site melanomas and 65% (13/20) of melanoma metastases had increased Akt3 expression. In contrast, only negligible fluctuations were observed when comparing expression of Akt2 in tumors versus melanocytes (FIG. 2D). Next, levels of activity were measured by quantifying phosphorylated Akt in either Akt3 or Akt2 immunoprecipitates. Strikingly, phosphorylated (active) Akt3 was detected in 62±0.02% (SE) of the samples (FIG. 2E). In contrast, no phosphorylated Akt2 (except for the positive control) was detected in these tumors. Furthermore, ˜35% of tumors had elevated Akt3 activity in comparison to melanocytes grown in culture. These data confirm the involvement of Akt3 deregulation in >60% of tumors from advanced-stage melanoma patients and suggest that increased expression is one of the mechanisms contributing to deregulated Akt3 activity in melanomas.

### Example 8

### Mechanisms Underlying Akt3 Deregulation in Melanomas

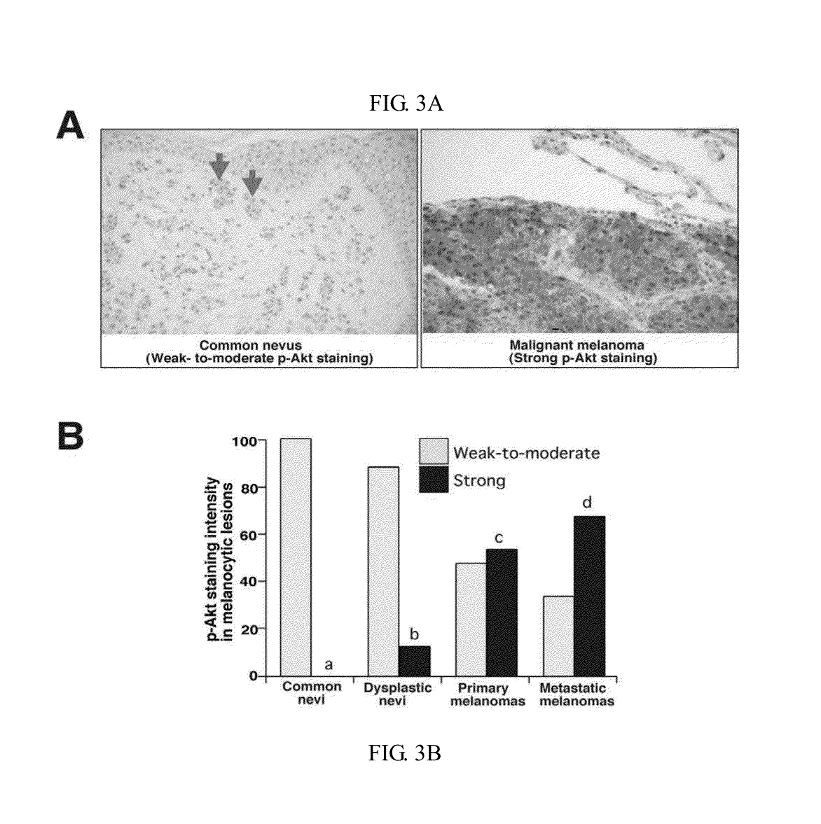

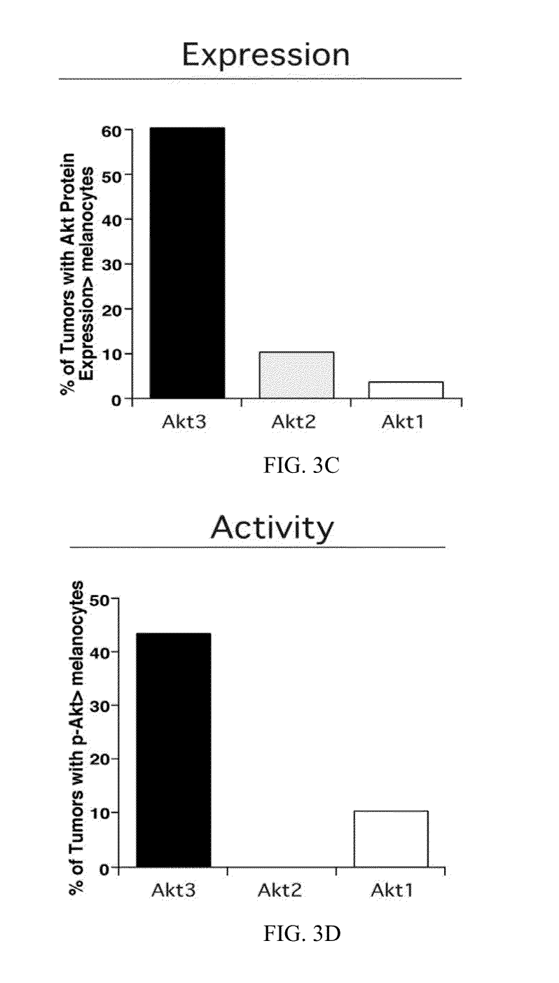

The foregoing experiments identified Akt3 as the predominantly active isoform in vitro in cell culture models and in vivo in patient tumors. Therefore, we next focused on determining the mechanisms leading to deregulated Akt3 activity in melanomas. Since the initial UACC 903 (PTEN) tumorigenesis model suggested that PTEN played a significant role regulating Akt activity in melanomas, we examined whether decreased expression of PTEN directly and specifically increased Akt3 activity. To accomplish this objective, PTEN expression (activity) was knocked down by siRNA in melanocytes and radial growth phase primary melanoma cells (WM35) to measure the effect on the level of phosphorylated Akt. The WM35 cell line was chosen since these cells have negligible basal Akt3 activity and express PTEN protein (see FIG. 1C). As predicted, FIG. 3A and FIG. 3B show that siRNA-mediated down regulation of PTEN led to an increase in total phosphorylated Akt (lanes 4 and 11), while a scrambled siRNA control exerted a negligible, non-significant, effect (lanes 2 and 9). The predominant Akt isoform activated following PTEN down regulation was determined by co-nucleofection of siRNA against PTEN together with either siRNA to Akt1 (lanes 5 and 12), Akt2 (lanes 6 and 13) or Akt3 (lanes 7 and 14). Only siRNA directed against Akt3 (lanes 7 and 14) lowered the level of phosphorylated Akt to that observed in non-nucleofected cells (lanes 1 and 8) or cells nucleofected with scrambled siRNA only (lanes 2 and 9). In contrast, reduction of Akt1 or Akt2 protein levels did not reduce the amount of phosphorylated Akt, again attesting to the selectivity of the Akt3 deregulation. Hence, selective regulation of Akt3 activity by PTEN is a significant mechanism for activating Akt3 in melanomas, since PTEN loss increases Akt3 activity without overexpression. Thus, a reduction in PTEN could, in turn, lead to an increase in the cellular PIP₃ (phosphatidylinositide 3,4,5-trisphosphate) concentration, which would be effective for specifically increasing Akt3 activity in melanomas. The mechanism underlying this specificity is currently unknown.

Studies involving tumor material from melanoma patients indicated that increased expression of Akt3 might also play a significant role augmenting Akt3 activity in melanomas. To investigate this possibility, Akt3 was overexpressed in melanocytes and WM35 cells (not shown) which express PTEN protein. HA-tagged wild type Akt3, a kinase dead version of Akt3 T305A/S472A (inactive) or a myristoylated Akt3 (active) was overexpressed in melanocytes (FIG. 3C). Cells were then starved of growth factors for 24 h, replenished with complete media and then lysates harvested 10 m later. Equivalent constructs for Akt2 were used as controls (data not shown). Overexpression of wild type Akt3 and myristoylated Akt3 led to increased levels of phosphorylated total Akt; in comparison to vector only or cells nucleofected with kinase dead Akt3. Furthermore, siRNA-mediated knockdown of PTEN together with Akt3 overexpression led to higher levels of phosphorylated Akt compared to wild type Akt3 expression alone (data now shown). Thus, overexpression of Akt3 alone, or in combination with PTEN loss, is an additional mechanism contributing to elevated Akt3 activity in melanomas.

### Example 9

### Increased Akt3 Activity Promotes Melanoma Tumorigenesis by Decreasing Apoptosis

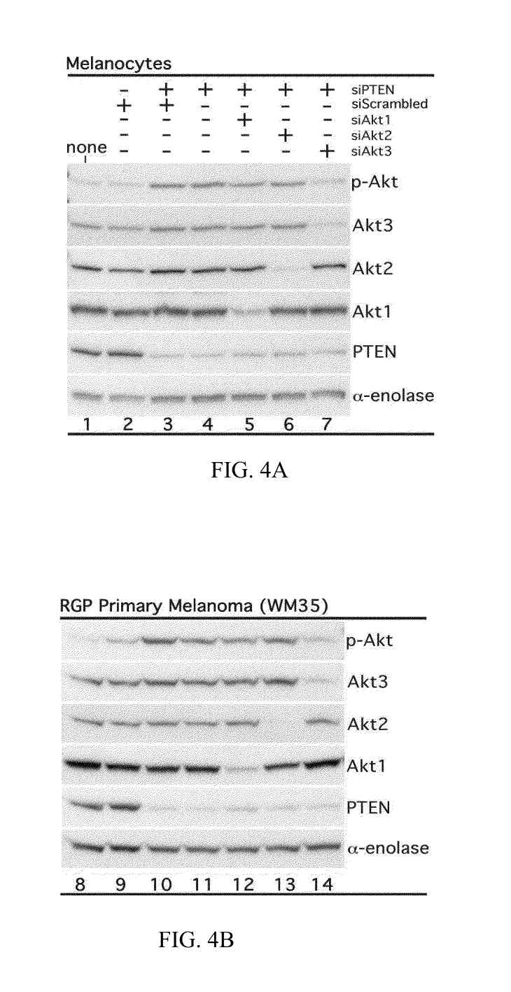

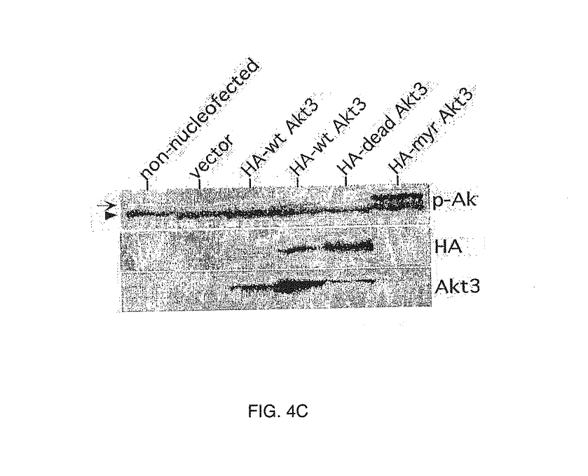

Since deregulated Akt3 activity was observed consistently in melanoma tumors, subsequent studies focused on determining the mechanisms by which increased Akt3 activity promoted tumorigenesis. Cell lines from UACC 903 (PTEN) tumorigenesis model were used to demonstrate that elevated Akt3 activity promoted melanoma tumorigenesis in a nude mouse model. One million cells from the parental. UACC 903 (-PTEN), 36A (+PTEN) or a 36A revertant (-PTEN) cell line were injected beneath the skin of 4- to 6-week old female nude mice and the size of the tumor formed was measured 10 days later. FIG. 4A shows that 36A cells having reduced Akt3 activity were non-tumorigenic in comparison with parental UACC 903 and revertant 36A cells having elevated Akt3 activity (P<0.5). While the tumorigenic potential of the 36A revertant cells increased significantly compared to 36A cells, tumor development remained delayed due to retention of a second melanoma suppressor gene on chromosome 10 that was used to create this model (Robertson et al., Cancer Res 59:3596-3601 (1999)). To confirm these observations and demonstrate the specificity of Akt3 involvement in melanoma tumorigenesis, we created a UACC 903 (Akt) model using siRNA. SiRNA-mediated reduction of Akt3 expression (activity) in UACC 903 cells, shown in FIG. 4B, significantly slowed tumor development in comparison to cells nucleofected with only buffer, scrambled siRNA or siRNA against Akt2 or Akt1 (P<0.05). Thus, either specifically reducing Akt3 activity using siRNA against Akt3 (FIG. 4B) or increasing PTEN expression (FIG. 4A) inhibited melanoma tumor development in nude mice.

To establish whether increased apoptosis was the predominant mechanism underlying tumor inhibition in vivo following decreases in Akt activity (Stahl et al., Cancer Res 63:2891-2897 (2003)), apoptosis was examined in both the UACC 903 (PTEN) (FIGS. 4C, 4E) and UACC 903 (Akt) models (FIGS. 4D, 4F) differing in Akt3 activity. Non-tumorigenic 36A and tumorigenic UACC 903 and 36A revertant cell lines were injected subcutaneously into nude mice and temporally and spatially matched tumor masses developing in parallel from each cell type were then harvested 4 days later to compare the magnitude of apoptosis, assessed by TUNEL (Stahl et al., Cancer Res 63:2891-2897 (2003)). A significantly greater number of apoptotic cells were observed in 36A (+PTEN) tumor masses having low Akt3 activity than in tumors formed from the parental UACC 903 (-PTEN) or 36A revertant (-PTEN) cell lines, which have high Akt3 activity (FIGS. 4C, 4E) (P<0.05). Similar results were observed in UACC 903 cells in which siRNA against Akt3 was used to lower Akt3 expression (activity). Cells nucleofected with buffer only or siRNA against Akt2 had approximately 5 to 7-fold fewer apoptotic cells than UACC 903 cells treated with siRNA against Akt3 (FIGS. 4D, 4F) (P<0.05). Thus, these results demonstrate that Akt3 activity preferentially regulates the extent of apoptosis, thereby aiding melanoma cell survival and promoting tumorigenesis.

### Experimental Discussion for Akt3

In the present invention, the Inventors demonstrate that Akt3 is an important survival kinase, in part, responsible for melanoma development. The UACC 903 (PTEN) melanoma model that reflected the importance of Akt in melanoma tumorigenesis was used to identify Akt3 as the predominant isoform deregulated during melanoma tumorigenesis. The use of siRNA demonstrated that selective knockdown of Akt3, but not Akt1 or Akt2, decreased the level of total phosphorylated Akt and lowered the tumorigenic potential of melanoma cells. Similar results were found in two independently derived melanoma cell lines (WM115 and SK-MEL-24), further supporting the significance of this discovery. The clinical relevance of this observation was validated by demonstrating that selective inhibition of Akt3 expression (by siRNA knockdown) or activity (by PTEN expression) significantly reduced melanoma tumor development.

Two distinct mechanisms leading to Akt3 activation in melanomas were identified in this study. The first mechanism is dependent upon overexpression of the structurally normal Akt3 protein. Analysis of advanced stage melanomas from human patients showed increased expression in >60% of the cases. Overexpression of Akt3 in melanocytes and WM35 cells lead to increased activity confirming the human tumor results. Overexpression of Akt is not unique to melanomas but has been documented in several human cancers with a number of studies reporting amplifications of the Akt isoforms. Amplification of Akt1 has been reported in stomach cancer (Staal, S. O., Proc Nat Acad Sciences USA 84:5034-5037 (1987)) while Akt2 gene amplification has been found in cancers of the ovary, pancreas, stomach and breast (Cheng et al., Proc Nat Acad Sciences USA 89:9267-9271 (1992); Chent et al., Proc Nat Acad Sciences USA 93:3636-3641 (1996); Lu et al., Chung-Hua I Hsuch Tsa Chih [Chinese Medical Journal] 75:679-682 (1995); Bellacosa et al., Int J Cancer 64:280-285 (1995); van Dekken et al., Cancer Res 59:748-752 (1999)). While no amplifications of the genomic regions containing the Akt genes have been reported in melanomas, several reports describe copy number increases of the long arm of chromosome 1 containing the Akt3 gene (Bastian et al., Cancer Res 58:2170-2175 (1998); Thompson et al., Cancer Genet Cytogenet 83:93-104 (1995); Mertens et al., Cancer Res 57:2765-2780 (1997)). In contrast, the long arms of chromosome 14 and chromosome 19 containing the Akt1 and Akt2 genes, respectively, which tend to be unchanged or undergo loss (Bastian et al., Cancer Res 58:2170-2175 (1998); Thompson et al., Cancer Genet Cytogenet 83:93-104 (1995); Mertens et al., Cancer Res 57:2765-2780 (1997)). Thus, copy number increases of the Akt3 gene is one of the mechanisms contributing to increased expression and activity of Akt3 in melanoma development.

The second mechanism identified that selective Akt3 activation in the UACC 903 (PTEN) model was due, in part, to decreased PTEN activity. A related observation in melanocytes and primary melanoma cells that retain PTEN expression (WM35) showed that siRNA-mediated reduction of PTEN specifically increased Akt3 phosphorylation (activity), further reinforcing the significance of Akt3 involvement in melanoma development. Published studies that characterize the genetic changes occurring in tumor material obtained from melanoma patients provide additional support for decreased PTEN expression playing a significant role in early melanoma development (Bastian et al., Cancer Res 58:2170-2175 (1998); Thompson et al., Cancer Genet Cytogenet 83:93-104 (1995); Mertens et al., Cancer Res 57:2765-2780 (1997); Parmiter et al., Cancer Genet Cytogenet 30:313-317 (1988)). Specifically, loss of one allele of PTEN, or PTEN haploinsufficiency, occurs commonly in early melanomas through loss of entire copy of chromosome 10 (Bastian et al., Cancer Res 58:2170-2175 (1998); Thompson et al., Cancer Genet Cytogenet 83:93-104 (1995); Mertens et al., Cancer Res 57:2765-2780 (1997); Parmiter et al., Cancer Genet Cytogenet 30:313-317 (1988)). Under this condition, it is predicted that loss of chromosome 10 reduces PTEN expression in a sub-population of evolving melanoma cells leading to increased Akt3 activation, providing these cells with a selective growth and survival advantage. Therefore, decreased expression due to haploinsufficiency or loss of activity of PTEN in melanoma plays an important role in melanoma tumor progression by specifically increasing Akt3 activity.

The underlying molecular basis for selective Akt3 activation, over Akt1 and Akt2, following decreased PTEN expression in melanomas is unknown. However, we speculate that the mechanism leading to this specificity involves preferential interaction of PIP₃ or other proteins with the pleckstrin homology (PH) domain. The amino-terminal PH domain mediates protein-protein and PIP₃ lipid-protein interactions. The PH domain of human Akt3 is ˜104 amino acids long (NCB1 accession number: NP_{—}005456) and 84% and 78% identical to Akt1 and Akt2, respectively (Brazil et al., Cell 111:293-303 (2002); and Nicholson et al., Cell Signal 14:381-395 (2002)). Furthermore, within the PH domain are phosphorylation sites that differ between the Akt isoforms and have as yet uncharacterized functions. For example, a ceramide-induced, PKC zeta-dependent, phosphorylation site at threonine 34 (within the PH domain), leads to inactivation of Akt1 by preventing binding to PIP₃ (Powell et al., Mol Cell Biol 23:7794-7808 (2003)). On the other hand, Akt2 and Akt3 have a serine at this position, which may be phosphorylated and regulated differently. Our analysis of other potential phosphorylation sites within the PH domain of the three Akt isoforms identified three potential unique Akt3 sites. Residue 21 of Akt3 is an asparagines while the equivalent sites on Akt1 and Akt2 are threonines. Furthermore, threonine 31 and tyrosine 49 of Akt3 were also found to differ from the other two Akt isoforms (Asn31 and Ser31 of Akt1 and Akt2, respectively; Ala50 and Pro50 of Akt1 and Akt2, respectively). Thus, differential regulation of putative phosphorylation sites within the PIP₃ lipid binding PH domain may offer a basis for the specificity of Akt3 activation in melanomas. It is also possible that unsuspected interactions between known oncogenes might be selectively regulating Akt isoform activation in melanomas. For example, TCL1 has been shown to selectively bind the Akt3 PH domain and promote hetero-oligomerization of Akt1 with Akt3 leading to transphosphorylation of the Akt molecules in leukaemogenesis (Laine et al., J Biol Chem 277:3743-3751 (2002)). TCL1 or other uncharacterized factors in melanoma cells may promote selective Akt3 activation in a similar manner.

Increased Akt3 activation also plays a significant role in the progression to more advanced aggressive tumors. Examination of Akt3 expression and activity in metastatic melanomas indicated that deregulated expression or activity occurs in >60% advanced stage metastatic melanomas. However, it is currently unknown whether the presence of elevated Akt3 activity can predict disease prognosis or the outcome of therapeutic regimes. Measurement of Akt3 activation in melanomas offers hope as a novel, more accurate prognostic indicator of disease outcome than the histopathologic measurements such as Breslow depth (i.e., the distance measured in millimeters from the granular cell layer to the deepest tumor cell) and ulceration (i.e., loss of the epidermis overlying the melanoma) that are currently used. A molecular based test assessing the activation state of Akt3 in melanocytic lesions may be more sensitive and less subjective than histological evaluation. This approach might also be useful for selecting appropriate patients for clinical trials utilizing drugs that are designed to target activated Akt3 or other members of this signaling pathway.

This study has shown that use of siRNA or expression of PTEN to lower Akt3 activity can effectively reduce the tumorigenic potential of melanoma cells by altering apoptotic sensitivity. Thus, melanoma cells having high levels of Akt3 activity are better suited for surviving in the in vivo tumor environment and inhibition of Akt3 activity, either directly or by interfering with its upstream regulators, is likely to represent an effective anticancer strategy for melanoma patients (Soengas et al., Oncogen 22:3138-3151 (2003); Johnstone et al., Cell 108:153-164 (2002)). Indeed, as the vast majority of chemotherapeutic agents work by inducing apoptosis, one would predict that inhibition of Akt3 could lower the threshold doses of drugs or radiation required for effective chemo- or radio-therapy, providing a mechanism to selectively target melanoma cells (Soengas et al., Oncogen 22:3138-3151 (2003)). Therefore, therapeutically targeting Akt3 activity alone or in combination with chemotherapeutic agents could be a potentially important therapy for melanoma patients (Soengas et al., Oncogen 22:3138-3151 (2003)). In summary, we have identified Akt3 as a specific prosurvival kinase, whose increased activity in melanoma tumors correlates with tumor progression and provides cells with a selective advantage to proliferate and survive environmental stresses.

### Examples for B-Raf

### Materials and Methods

### Example 10

### Cell Lines, Culture Conditions and B-Raf Mutational Status

The human melanoma cell lines UACC 903, 1205 Lu and C8161, as well as HEK 293T cells were maintained in DMEM (Invitrogen, Carlsbad, Calif.) supplemented with 10% FBS (Hyclone, Logan, Utah). The presence or absence of the T1796A B-RAF mutation in the UACC 903 and C8161 cell lines was undertaken as described previously (Miller C J et al. J Invest Dermatol. 123: 990-2 (2004)). Furthermore, the presence of this mutation in UACC 903 and 1205 Lu cells has been reported previously (Miller C J et al. J Invest Dermatol. 123: 990-2 (2004)., Tsao H et al. J Invest Dermatol. 122: 337-41 (2004)., Krasilnikov M et al. Oncogene. 22: 4092-101 (2003).).

### Example 11

### In Vitro SiRNA Studies

SiRNA (100 pmol) was introduced into 1×10⁶ UACC 903, 1205 Lu or C8161 cells via nucleofection with an Amaxa Nucleofector (Koeln, Germany) using Solution R/program K-17 as described in ref (Stahl J M et al. Cancer Res. 64: 7002-10 (2004).). The resultant transfection efficiency was >90%. Following nucleofection, cells were replated for 24-48 hours after which protein lysates were harvested for Western blot analysis. To measure the duration of siRNA knockdown, cells were harvested at 0, 2, 4, 6, and 8 days following nucleofection with siRNA to B-Raf or C-Raf and subjected to Western blot analysis. Duplexed Stealth siRNA (Invitrogen, Carlsbad, Calif.) were used for these studies with the B-Raf sequences modified from ref. (Hingorani S R et al. Cancer Res.; 63: 5198-202 (2003).). The siRNA sequences used were as follows: WT B-RAF (COM4 or 4)-GGACAAAGAAUUGGAUCUGGAUCAU (SEQ ID NO:11); MUT B-RAF (MuA or A)-GGUCUAGCUACAGAGAAAUCUCGAU (SEQ ID NO:10); C-RAF-GGUCAAUGUGCGAAAUGGAAUGAGC (SEQ ID NO:12); LAMIN A/C-GAGGAACUGGACUUCCAGAAGAACA (SEQ ID NO:13); and VEGF-GCACATAGGAGAGATGAGCTTCCTA (SEQ ID NO:14).

### Example 12

### Western Blot Analysis

For Western Blot analysis, cell lysates were harvested in petri dishes by the addition of lysis buffer containing 50 mM HEPES (pH 7.5), 150 mM NaCl, 10 mM EDTA, 10% glycerol, 1% Triton X-100, 1 mM sodium orthovandate, 0.1 mM sodium molybdate, 1 mM phenylmethylsulfonyl fluoride, 20 μg/ml aprotinin, and 5 μg/ml leupeptin. Whole cell lysates were centrifuged (≧10,000×g) for 10 minutes at 4° C. to remove cell debris. Proteins were quantitated using the BCA Assay from Pierce (Rockford, Ill.), and 30 μg of lysate per lane were loaded onto a NuPage Gel Life Technologies, Inc. (Carlsbad, Calif.). Following electrophoresis, samples were transferred to polyvinylidene difluoride membrane (Pall Corporation, Pensacola, Fla.). The blots were probed with antibodies according to each supplier's recommendations: anti-pErk and anti-pMek from Cell Signaling Technologies (Beverly, Mass.); antibodies to B-Raf, C-Raf, Erk2 and α-enolase from Santa Cruz Biotechnology (Santa Cruz, Calif.); and an antibody to Lamin A/C from Biomeda Corp (Foster City, Calif.). Secondary antibodies were conjugated with horseradish peroxidase and obtained from Santa Cruz Biotechnology. The immunoblots were developed using the enhanced chemiluminescence detection system (Amersham Pharmacia Biotech, Piscataway, N.J.).

### Example 13

### In Vivo SiRNA Studies

Animal experimentation was performed according to protocols approved by the Institutional Animal Care and Use Committee at The Pennsylvania State University College of Medicine. Tumor kinetics were measured by subcutaneous injection of 1×10⁶ UACC 903 or 1205 Lu cells nucleofected with siRNA in 0.2 ml of DMEM supplemented with 10% FBS above both the left and right rib cages of six, 4-6 week old nude mice (Harlan Sprague Dawley, Indianopolis, Ind.). The dimensions of developing tumors were measured using calipers on alternate days. For mechanistic studies, 5×10⁶ UACC 903 cells nucleofected with siRNA were injected into mice and tumors harvested 4 days post injection of cells in order to measure changes in cell proliferation and apoptosis, as described previously (Stahl J M et al. Cancer Res. 64: 7002-10 (2004)., Stahl J M et al. Cancer Res. 63: 2881-90 (2003).).

### Example 14

### In Vitro and In Vivo BAY 43-9006 Studies

The BAY 43-9006 compound used for these studies was synthesized as described in ref. (Bankston D et al. Organic Process Res Dev. 6: 777-81 (2002).). To evaluate the inhibitory effects of BAY 43-9006 on wild type and mutant B-Raf, HEK 293T cells were transfected with HA-tagged wild type B-RAF, mutant ^{V599E}B-RAF or vector (pcDNA3) using Calcium Phosphate as described previously (Robertson G P et al. Proc Natl Acad Sci USA. 95: 9418-23 (1998).). Following transfection (72 hours) media was replaced with DMEM media supplemented with 10% FBS and 5 uM BAY 43-9006 or DMSO vehicle. Two hours later, protein lysates were collected for Western blot analysis. Levels of phosphorylated Mek and Erk were quantified from 3 independent blots and fold differences under different conditions were estimated after normalizing against an Erk 2 loading control.

Effect of BAY 43-9006 on tumor development was measured by subcutaneously injecting 5×10⁶ UACC 903 or 1×10⁶ 1205 Lu cells into nude mice. After 6 days when a small tumor (50-100 mm³) had developed, the mice received an intra peritoneal injection on alternate days consisting of 50 μl of vehicle (DMSO), or the drug BAY 43-9006 at concentrations of 10, 50 or 100 mg/kg body weight for UACC 903 cells and 50 mg/kg body weight for 1205 Lu cells. For studies involving pretreatment with BAY 43-9006, 50 mg/kg body weight of drug was intra peritoneally injected twice (−4 and −2 days) prior to subcutaneous injection of UACC 903 or 1205 Lu cells. The mechanism by which pharmacological inhibition of mutant ^{V599E} B-Raf delays tumor development was identified by comparing tumors of the same size developing in parallel. This was achieved by subcutaneous injection of 5×10⁶ UACC 903 cells followed at day 6 by intra peritoneal injection every 2 days with 50 mg/kg of BAY 43-9006. For temporal and spatial matching of control DMSO with drug treated tumors, either 1×10⁶, 2.5×10⁶ or 5×10⁶ million UACC 903 cells were subcutaneously injected and from day 6 treated intra peritoneally with DMSO vehicle every 2 days. Drug or vehicle treated tumors of the same size developing in parallel were harvested at days 9, 11, 13 and 15 for comparison. At each time point, tumors from mice treated with vehicle or drug were harvested for analysis of cell proliferation, apoptosis and vascular development, as described previously (Stahl J M et al. Cancer Res. 64: 7002-10 (2004), Stahl J M et al. Cancer Res. 63: 2881-90 (2003).).

### Example 15

### Apoptosis, Cell Proliferation and Vessel Density Measurements in Tumors

Apoptosis measurements on formalin-fixed, paraffin-embedded tumor sections were undertaken using the TUNEL TMR Red Apoptosis kit from Roche (Manheim, Germany), as described previously (Stahl J M et al. Cancer Res. 64: 7002-10 (2004), Stahl J M et al. Cancer Res. 63: 2881-90 (2003).). Cell Proliferation rates in formalin-fixed tumor sections were measured using the RPN 20 cell proliferation kit (Amersham Biosciences, Piscataway, N.J.) that utilizes BrdU incorporation and immunocytochemistry. Two hours prior to sacrificing, 0.2 ml of BrdU was injected intra peritoneally into mice and tumors processed according to the proliferation kit's instructions. The number of BrdU stained cells were scored as the percentage of total cells of tumors treated with BAY 43-9006 or vehicle (DMSO). Quantification of vessels density using a purified rat anti-mouse CD31 (PECAM-1) monoclonal antibody (Pharmingen, San Diego, Calif.) has been described previously (Stahl J M et al. Cancer Res. 64: 7002-10 (2004), Stahl J M et al. Cancer Res. 63: 2881-90 (2003).). The proportional area of the tumors occupied by the vessels over the total area was calculated using the IP Lab imaging software program. For all tumor analyses, a minimum of 6 different tumors with 4-6 fields per tumor was analyzed and results represented as the average±SEM.

### Example 16

### In Vivo pErk Measurements

To quantitate changes in pErk levels in formalin-fixed, paraffin-embedded tumor sections, antigen retrieval was performed with 0.01 M citrate buffer at pH 6.0 for 20 minutes in a 95° C. water bath. Slides were cooled for 20 minutes, rinsed in PBS and then incubated in 3% H₂O₂ for 10 minutes to quench endogenous peroxidase activity. Next, sections were blocked with 1% BSA for 30 minutes and incubated with anti-pERK antibody at a 1:100 dilution (Cell Signaling Technologies, Beverly, Mass.) overnight at 4° C. Following rinsing in PBS, sections were incubated with biotinylated anti rabbit IgG for 1 hour, rinsed again in PBS, and incubated with peroxidase labeled streptavidine for 30 minutes. Visualization was accomplished using the AEC (aminoethyl carbazole) substrate kit for 5-10 minutes (Zymed laboratories Inc., South San Francisco, Calif.) and nuclei counterstained with hematoxylin prior to coverslip mounting using an aqueous mounting solution. The average percentage of cells ±SEM that stained positive for pErk was counted from a minimum of 6 different tumors with 4-6 fields counted per tumor.

### Example 17

### In Vitro Doubling Times and In Vivo Tumor Latency Periods

The in vitro doubling time of UACC 903 cells nucleofected with siRNA was estimated by plating 5×10³ cells/well in 200 ul of DMEM supplemented with 10% FBS in multiple rows of wells in five 96-well plates. Growth was measured every 24 hours over a period of 5 days by performing a colorimetric assay on one plate each day using the Sulforhodamine B (SRB) binding assay (Sigma Chemical Co., St Louis, Mo.) and the doubling time calculated, as described previously (Stahl J M et al. Cancer Res. 63: 2881-90 (2003).). The in vivo tumor latency period was measured by estimating number of days required for mean tumor size to reach 10 mm³.

### Example 18

### BAY 43-9006 Growth Inhibition/IC-50 of UACC 903 Melanoma Cells

To measure the growth inhibitory effects or IC-50 of BAY 43-9006 on UACC 903 cells, 5×10³ cells/well were plated into 96-well plates. Following 24 hours, varying concentrations of BAY 43-9006 (0, 0.02, 0.1, 0.4, 1.6, 6.3, 25, or 100 uM) was added to duplicate 8-strip wells in the plate. After 72 hours of growth at 37° C. in a 5% CO₂ humidified atmosphere, media was discarded and cells were fixed in 10% trichloroacetic acid. Surviving cells at each concentrations of the drug were calculated using the SRB binding assay (Stahl J M et al. Cancer Res. 63: 2881-90 (2003).). Western blot analysis was used to demonstrate the effects of increasing concentrations of BAY 43-9006 (5, 10, 15 or 20 uM) on phosphorylation levels of Mek 1/2 and Erk 1/2 in UACC 903 cells following 2 hours drug exposure.

### Example 19

### VEGF Expression Analysis

To determine the amount of VEGF secreted by cells following siRNA-mediated knockdown of B-Raf protein or after treatment with BAY 43-9006, the human VEGF Quantikine kit (DVE00) was used (R&D Systems Inc., Minneapolis, Minn.). UACC 903 or 1205 Lu cells (5×10³) nucleofected with the various siRNA were plated in 60 mm petri dishes and 24 hours later media replaced with DMEM containing 2% FBS. Following an additional 24 hours, media was again replaced and conditioned media for ELISA analysis was collected 24 and 48 hours later. For BAY 43-9006 studies, 3×10⁵ UACC 903 or 1205 Lu cells were plated into 60 mm petri dishes and 24 hours later media was changed to DMEM containing 2% FBS. After an additional 24 hours, media was replaced with DMEM supplemented with 2% FBS alone or in combination with BAY 43-9006 (5, 10, 15 uM) or DMSO vehicle. After 12 or 24 hours, conditioned media was collected for ELISA analysis. The media was cleared by centrifugation at 14,000 rpm (4° C.) for 5 minutes and stored at −80° C. VEGF ELISA analysis was performed in triplicate on duplicate experiments according to the manufacturer's instructions.

### Example 20

### Statistics

For statistical analysis, the Student's t-test was used for pairwise comparisons and the One-way Analysis of Variance (ANOVA) or the Kruskal-Wallis test was used for groupwise comparisons, followed by the appropriate post hoc tests (Dunnett's, Tukey's or Dunn's). Results were considered significant at a P-value of <0.05.

### Experimental Results

### Example 21

### SiRNA-Mediated Targeting of Mutant ^{V599E}B-Raf Inhibits Melanoma Tumor Development

TABLE 2
Growth properties of UACC 903 cells treated with
siRNA against B-Raf, C-Raf or scrambled siRNA

| | Doubling time | % of proliferating | Latent period for |
|---|---|---|---|
| SiRNA | in vitro | cells at day 4 | tumor formation |
| Treatment | in days (hours) | in tumors ± SEM | (days)¹ |
| Scrambled | 1.25 (30) | 10 ± 0.7 | 5 |
| C-Raf | 1.1 (26) | 15 ± 0.6 | 5 |
| B-Raf (4) | 1.6 (38.4) | 2 ± 0.6 | 14 |
| B-Raf (A) | 1.7 (40.8) | 2 ± 0.4 | 16 |

¹Latent Period for tumor formation was defined as the number of days required for mean tumor size to reach 10 mm³.

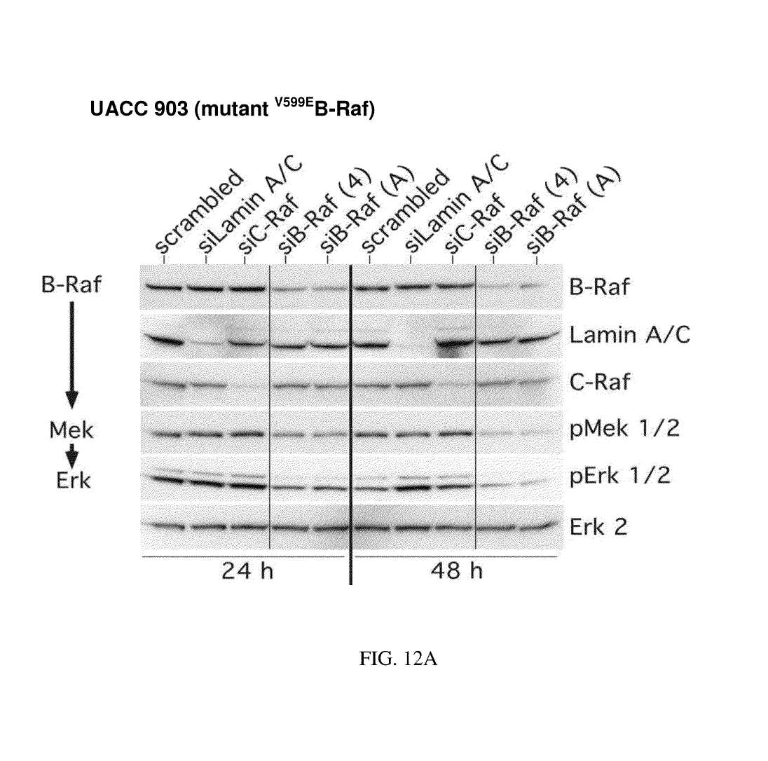

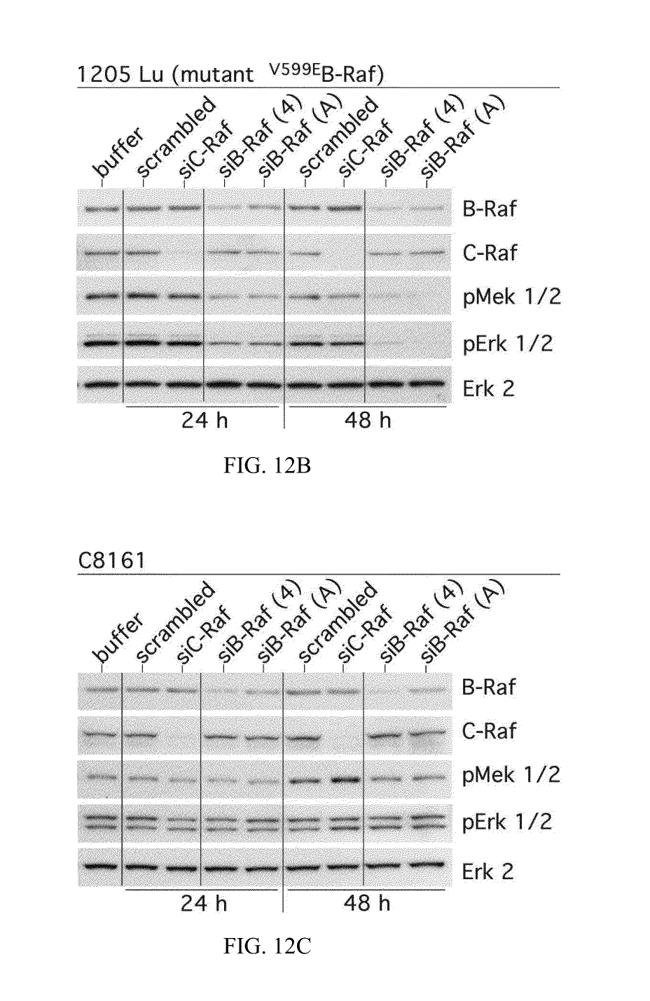

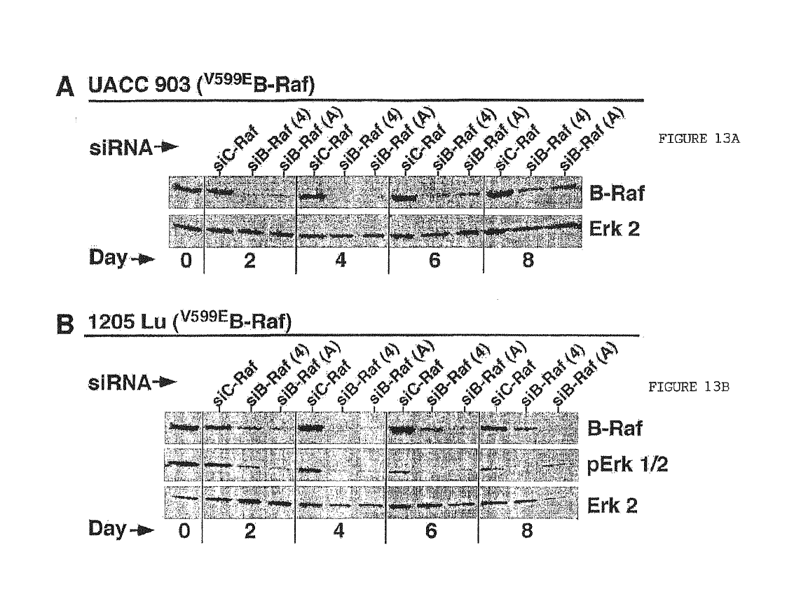

The role of mutant ^{V599E}B-Raf in melanoma tumorigenesis is currently unknown. To address this issue, we reasoned that inhibition of expression or activity of mutant ^{V599E}B-Raf protein could be used to identify the role this protein plays in melanoma tumorigenesis. An siRNA-mediated approach was used to knockdown expression of mutant ^{V599E}B-Raf in UACC 903 and 1205 Lu cell lines containing mutant protein or B-Raf in the C8161 cell line lacking the T1796A mutation. The MuA or A siRNA was designed to reduce expression of wild type and mutant protein while the Com4 or 4 siRNA only lowered expression of mutant protein as described previously (Hingorani S R et al. Cancer Res.; 63: 5198-202 (2003).). SiRNA for these studies was introduced into the cell lines via nucleofection resulting in transfection efficiencies of >90% (data not shown) (Stahl J M et al. Cancer Res. 64: 7002-10 (2004).). Effectiveness of siRNA for reducing the expression of B-Raf and C-Raf protein in UACC 903 (FIG. 12A), 1205 Lu (FIG. 12B) and C8161 (FIG. 12C) cells after nucleofection was measured by Western blot analysis. At 24 and 48 hours after nucleofection, each siRNA reduced only expression of the protein against which it was made, thereby demonstrating the specificity and effectiveness of the siRNA knockdown in each of these cell lines. In UACC 903 and 1205 Lu cells, only siRNA to B-Raf reduced phosphorylation (activity) levels of the downstream targets Mek and Erk, whereas scrambled siRNA or siRNA to C-Raf had no effect on these proteins (FIG. 12A and FIG. 12B). Maximal decrease in phosphorylation (activity) levels of Mek and Erk in UACC 903 and 1205 Lu cells were observed 48 hours after nucleofection. In contrast, reduced expression of B-Raf or C-Raf in C8161 cells had a negligible insignificant effect on levels of phosphorylated Mek and Erk (FIG. 12C). Thus, inhibition of ^{V599E}B-Raf expression in melanoma cell lines containing mutant protein leads to reduced activity of Mek and Erk, while lowering expression of B-Raf protein in melanoma cells lacking the T1796A mutation does not appear to affect activity of downstream targets.

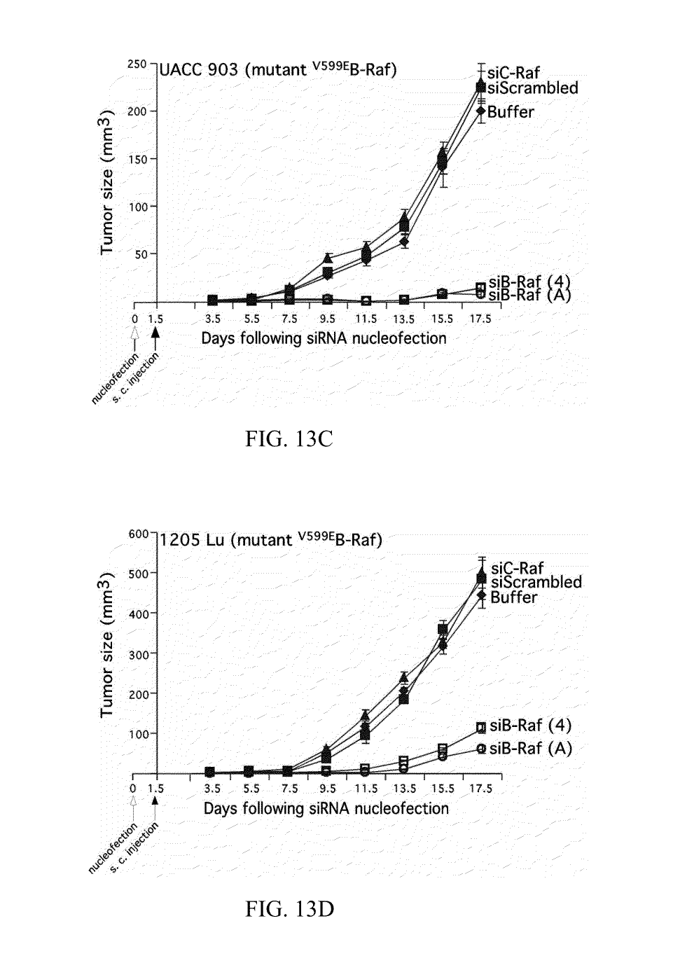

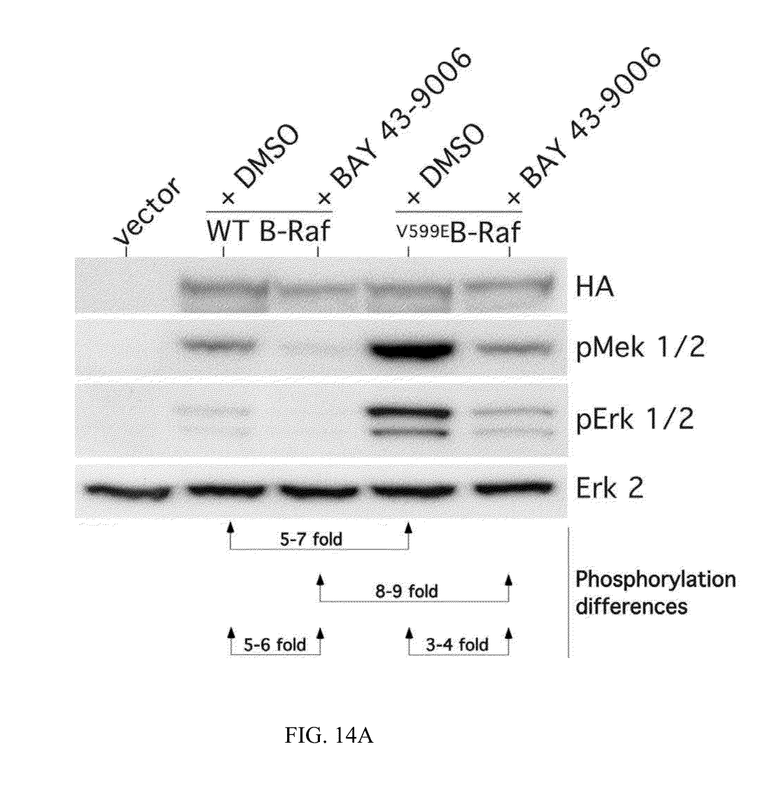

To measure the effect of reduced ^{V599E}B-Raf expression (activity) on melanoma tumor development, ^{V599E}B-Raf expression in UACC 903 and 1205 Lu cell lines was inhibited using siRNA followed by subcutaneous injection into mice using a transient knockdown approach that we have reported previously (Stahl J M et al. Cancer Res. 64: 7002-10 (2004).). SiRNA-mediated knockdown of protein expression persisted for a minimum of 8 days in UACC 903 (FIG. 13A) and 1205 Lu (FIG. 13B) cells. Furthermore, a corresponding decrease in pErk levels was also observed for the same period (FIG. 13B). The size of the developing tumor was measured on alternate days up to 17.5 days after nucleofection to determine the effect of B-Raf knockdown on melanoma tumorigenesis. A reduction in tumor development was observed in both UACC 903 (FIG. 13C) and 1205 Lu (FIG. 13D) cells in which mutant ^{V599E} B-Raf expression had been knocked down. In contrast, siRNA-mediated inhibition of C-Raf, a scrambled siRNA or buffer controls did not alter tumor development. Lack of an effect following knockdown of C-Raf, suggested that signaling through ^{V599E}B-Raf was specifically necessary for tumor development. Thus, siRNA-mediated reduction of ^{V599E}B-Raf expression (activity) in melanoma cells prior to injection into mice inhibited tumorigenesis.

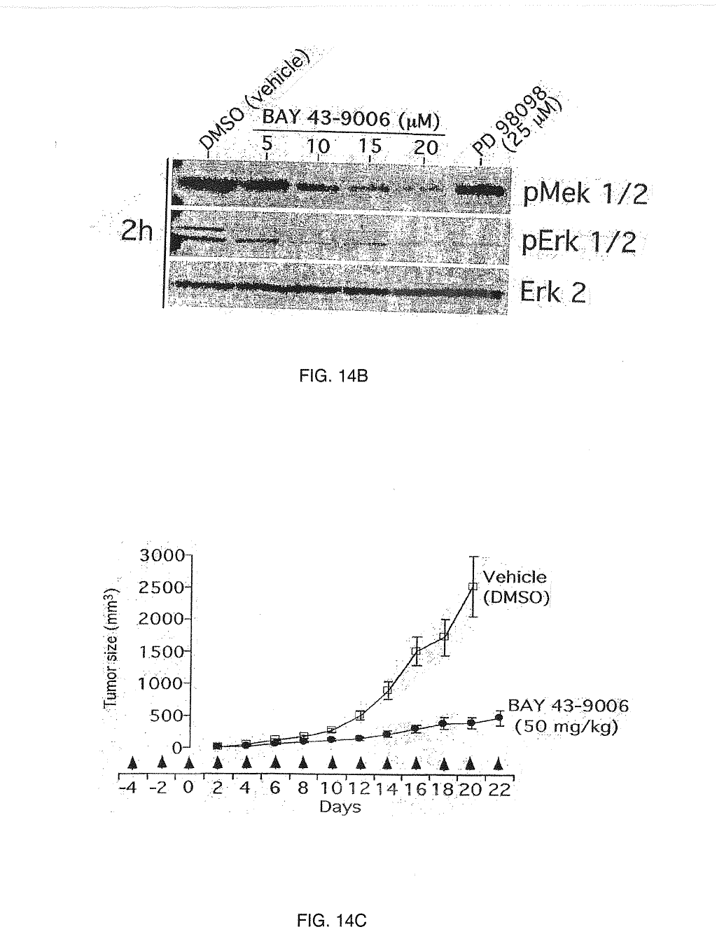

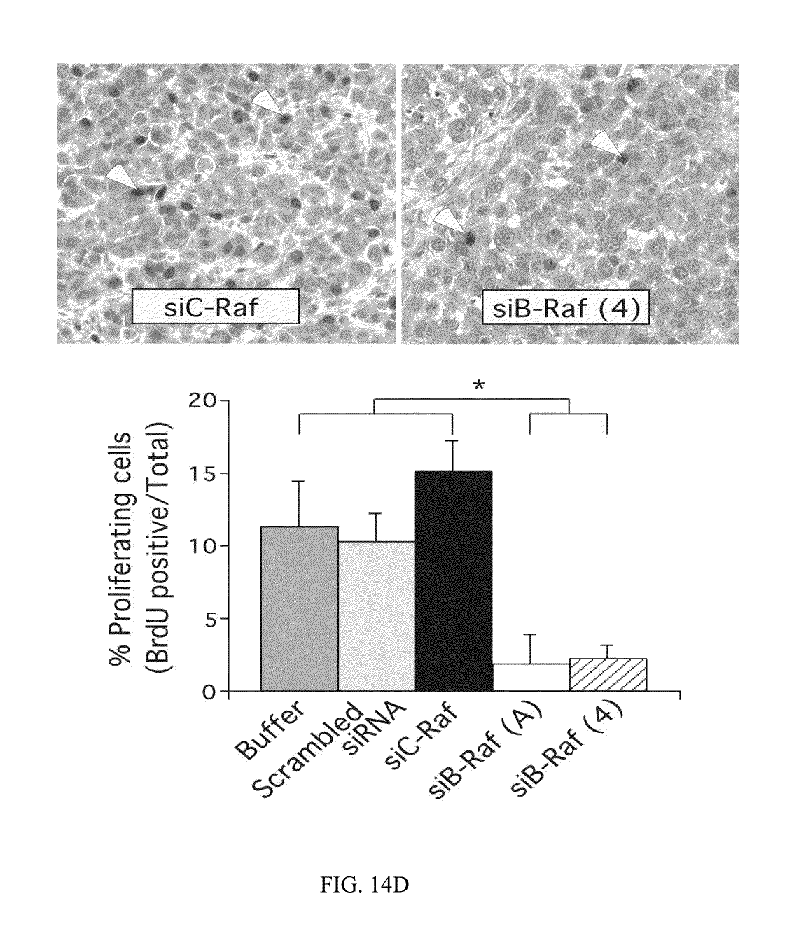

A similar experiment was undertaken using a Raf kinase inhibitor, called BAY 43-9006 to inhibit the activity of B-Raf protein in UACC 903, 1205 Lu or C8161 cells. This compound, originally identified in a screen for Raf kinase inhibitors, has been shown to effectively inhibit the activity of wild type B-Raf protein (Lowinger T B et al. Curr Pharm Des. 8: 2269-78 (2002)., Lyons J F et al. Endocr Relat Cancer. 8: 219-25 (2001).). Initially, we determined the concentration of BAY 43-9006 that reduced UACC 903 cell survival by half, also called the IC-50, and found it to be 5-6 uM (data not shown). Therefore, a concentration of 5 uM was chosen for subsequent in vitro studies. Next, we demonstrated that BAY 43-9006 inhibited activity of both mutant and wild type B-Raf protein to a similar extent by expressing either HA-tagged wild type or mutant ^{V599E}B-Raf constructs in HEK 293T cells (FIG. 14A). As reported previously, we observed levels of phosphorylated (active) Erk or Mek in cells expressing ^{V599E}B-Raf to be 5-7 fold higher than in cells transfected with only wild type B-RAF (Davies H et al. Nature. 417: 949-54 (2002).). HEK 293T cells expressing either wild type or mutant ^{V599E}B-Raf protein were then exposed to 5 uM BAY 43-9006 for 2 hours to examine the effect on the activity of the signaling pathway. Exposure to BAY 43-9006 reduced levels of phosphorylated Mek and Erk in cells expressing either wild type or mutant ^{V599E}B-Raf protein by 5-6 fold and 3-4 fold, respectively (FIG. 14A). Thus, BAY 43-9006 inhibits the activity of both wild type and mutant B-Raf.

To demonstrate that BAY 43-9006 inhibited mutant ^{V599E}B-Raf protein signaling in UACC 903 cells, in vitro cultures were exposed for 2 hours to increasing concentrations of BAY 43-9006. BAY 43-9006 reduced the levels of phosphorylated (active) Mek and Erk in UACC 903 cells in a dose responsive manner (FIG. 14B). The inhibitory effects of BAY 43-9006 on MAP kinase signaling persisted for at least 2 to 3 days in UACC 903 and 1205 Lu cell lines (data not shown). We next evaluated the effect of pretreating animals with BAY 43-9006 prior to subcutaneous injection of UACC 903 or 1205 Lu cells. For these experiments, mice were exposed to 50 mg/kg BAY 43-9006 for 4 days prior to subcutaneous injection of 5×10⁶ cells, which was followed by intra peritoneal injection of drug every 2 or 3 days up to day 22. Both UACC 903 (FIG. 14C) and 1205 Lu (not shown) tumor development was significantly inhibited (Student's t-test; p<0.05), and comparison of size matched UACC 903 tumors revealed reduced proliferation and decreased vascular development in BAY 43-9006 treated tumors compared to vehicle treated controls (not shown). Furthermore, tumor size increased slowly to day 8 after which it leveled off with no statistical difference between subsequent tumor measurements (ANOVA; P>0.05). Thus, pharmacological inhibition of mutant ^{V599E}B-Raf activity by pretreatment of the host animal with BAY 43-9006 significantly reduced the tumorigenic potential of melanoma cells expressing mutant ^{V599E}B-Raf.

To identify the mechanism leading to tumor inhibition in cells pretreated with siRNA to knockdown ^{V599E}B-Raf activity, rates of tumor cell proliferation and apoptosis were measured in UACC 903 tumors 4 days after subcutaneous injection. No difference in the rate of apoptosis (1-2%) was detected using the TUNEL assay (data not shown). However, UACC 903 cells treated with siRNA to B-Raf had 5 to 6-fold fewer proliferating cells compared to control cells nucleofected with buffer only, scrambled siRNA or C-RAF siRNA (FIG. 14D). Next, in vitro doubling times, in vivo proliferation rates and tumor latency periods of the UACC 903 cell line were compared to determine whether reduced growth could account for delayed tumor development (Table 2). UACC 903 cells nucleofected with siRNA to C-RAF or scrambled siRNA doubled in number in vitro every 1.2 days (or ˜29 h), whereas cells nucleofected with siRNA against B-RAF doubled every 1.65 days (or ˜40 h), which was a delay of ˜38%. In contrast, analysis of proliferating cells in tumors showed a significant difference between control tumors nucleofected with siRNA to C-Raf or scrambled siRNA (ANOVA; p<0.05), which had 10-15% proliferating cells, compared to tumors cells nucleofected with siRNA to B-Raf that had 2-3% proliferating cells. The ˜82% reduction in proliferative capacity of cells nucleofected with B-RAF siRNA could account for the delayed latency period of tumor development. Hence, for tumors of the same size as controls at day 5, cells nucleofected with siRNA to B-RAF required an additional 10 days to form tumors of the same size (Table 2). Since tumor development was delayed >200%, the reduced growth rate observed in vitro and in vivo could account for the reduced tumorigenic potential of these cells. Therefore, inhibition of mutant ^{V599E}B-Raf expression (activity) in melanoma cells prior to tumor formation significantly reduced the in vivo growth potential of cells, thereby delaying tumorigenesis.

### Example 22

### Inhibition of Melanoma Tumor Development by Targeting Mutant ^{V599E}B-Raf in Preexisting Tumors

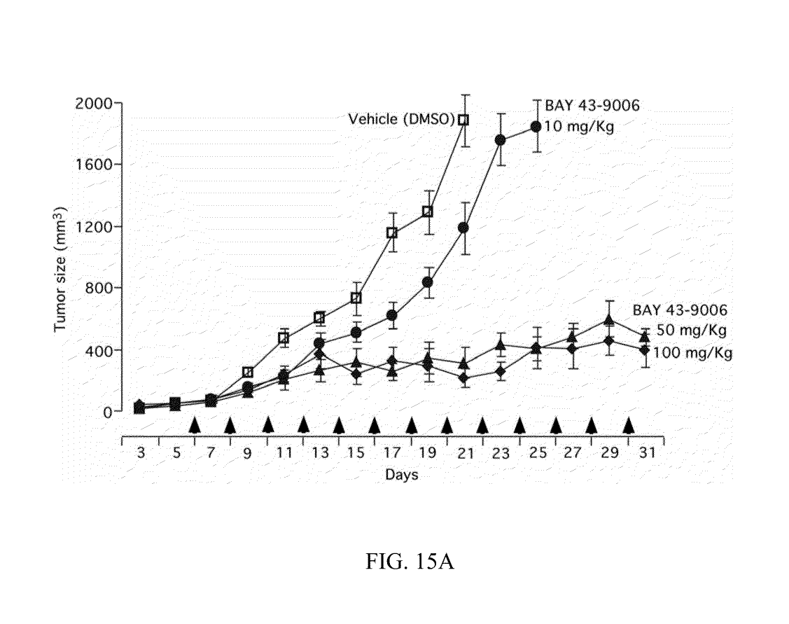

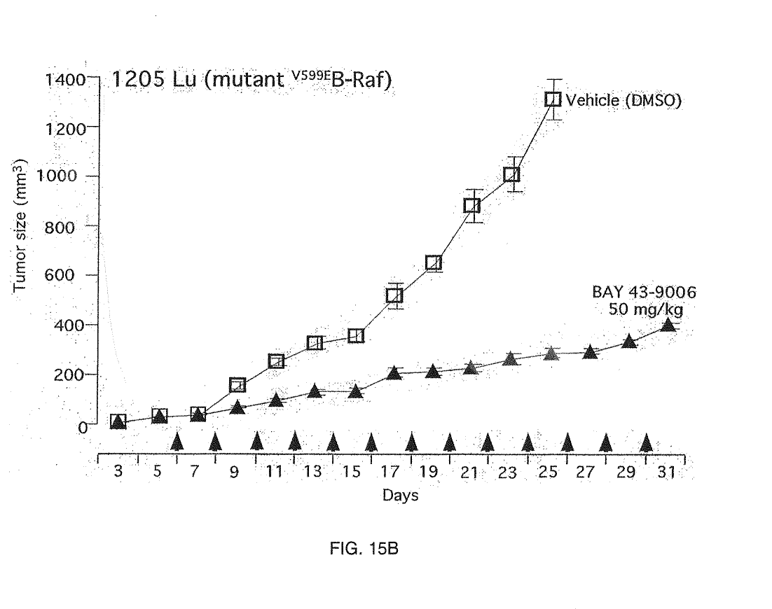

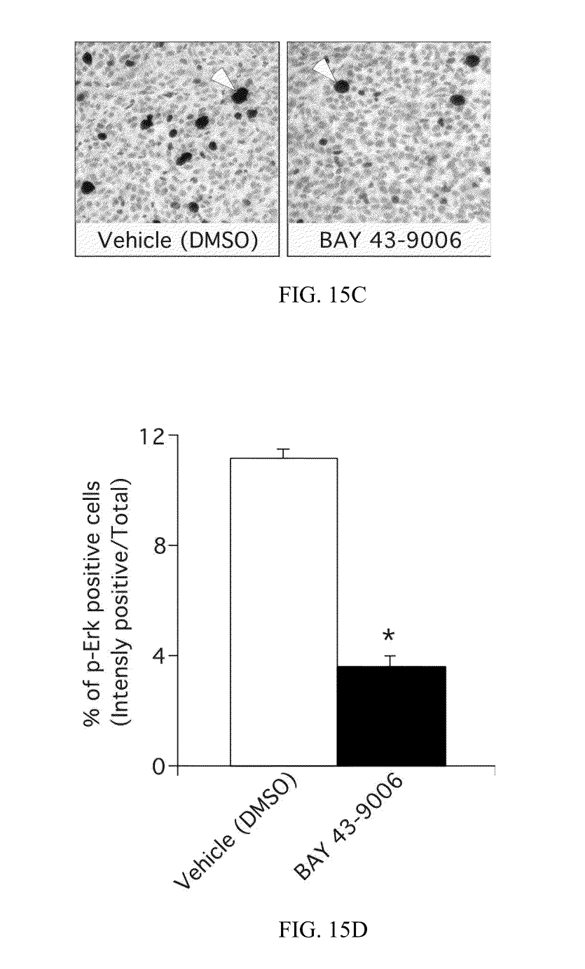

It is currently unknown whether targeting mutant ^{V599E}B-Raf in established preexisting melanoma tumors could retard tumor development, and if so, whether the mechanism is the same as that occurring when targeting ^{V599E}B-Raf in cells prior to tumor formation. Therefore, we next examined whether pharmacologically targeting B-Raf in preexisting melanoma tumors would inhibit tumor development by a similar mechanism. Five million UACC 903 cells, one million 1205 Lu cells or five million C8161 cells were subcutaneously injected into 4- to 6-week old female nude mice. On day 6, vehicle (DMSO) or BAY 43-9006 compound dissolved in vehicle (10, 50 or 100 mg/kg) was administered to mice via intra peritoneal injection every 48 hours. A 48 hour time period between drug administrations was chosen since inhibitory effects on the MAP kinase signaling pathway in UACC 903, 1205 Lu and C8161 cells persisted for at least that period (data not shown). Size of the developing tumors was measured using calipers on alternate days and the results are shown for UACC 903 cells in FIG. 4A and 1205 Lu cells in FIG. 15B. While all concentrations of the BAY 43-9006 compound slowed UACC 903 tumor development, only concentrations ≧50 mg/kg caused tumor development to plateau 7 days following the start of treatment (FIG. 4B). Tumor development in mice treated with BAY 43-9006 at 10 mg/kg was delayed ˜1 week, but UACC 903 tumors steadily increased in size and mice had to be euthanized on day 27 when tumors reached sizes >2,400 mm³. For UACC 903 cells, a small increase occurred in the size of the tumor up to day 13; however, after a week of drug treatment, tumor sizes stabilized and there was no statistically significant increase in tumor sizes from days 13-to-31 (FIG. 15A)(ANOVA; P>0.05). Treatment of 1205 Lu tumors with 50 mg/kg BAY 43-9006 also reduced tumor development in a similar manner causing a plateau in tumor size from days 17-to-31 (FIG. 15B))(ANOVA; P=0.12). In contrast, while BAY 43-9006 inhibited pMek and pErk levels in C8161 cells, no difference was observed in the kinetics of tumor formation (data not shown). Thus, pharmacological inhibition of mutant ^{V599E}B-Raf activity retards tumor development in preexisting melanoma tumors but does not cause tumor regression. In contrast, inhibition of B-Raf in melanoma cells lacking the T1796A mutation did not appear to alter tumorigenic potential.

To confirm that the BAY 43-9006 compound affected activity of the mutant ^{V599E}B-Raf signaling pathway in tumors, the percentage of cells expressing elevated levels of phosphorylated Erk was scored in tumors from mice 9 days after start of treatment with vehicle (DMSO) or vehicle containing 50 mg/kg BAY 43-9006 (FIG. 15C). Quantification of the number of pErk positive cells showed that BAY 43-9006 treated tumors had ˜3-fold fewer pErk positive cells than control vehicle treated tumors (FIG. 15D)(Student's t-test; P<0.05). The significantly greater number of phosphorylated Erk positive cells in vehicle treated tumors indicated that BAY 43-9006 was inhibiting the activity of the mutant ^{V599E}B-Raf signaling pathway in vivo. Thus, these results demonstrate that pharmacological inhibition of mutant ^{V599E}B-Raf with BAY 43-9006 reduces MAP kinase pathway signaling in tumors, thereby mediating tumor inhibition.

### Example 23

### Mechanistically, BAY 43-9006 Inhibits Vascular Development of Preexisting Melanoma Tumors Leading to Increased Apoptosis

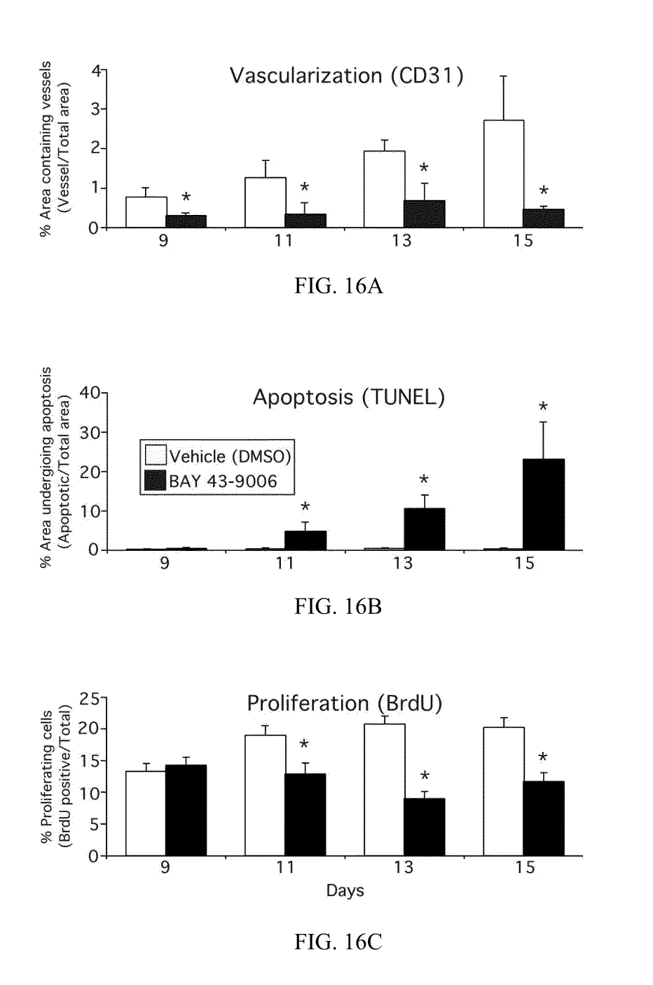

The foregoing experiments showed a consistent relationship between inhibition of mutant ^{V599E}B-Raf activity and reduced tumor development; therefore, subsequent studies focused on identifying the mechanism by which this occurred in existing melanoma tumors. For these studies, temporally and spatially matched UACC 903 tumors exposed to either vehicle or BAY 43-9006 were analyzed for vascular development as well as apoptosis and proliferation rates in order to identify the key event delaying growth of existing established tumors. Matched tumors were harvested every two days, starting at day 9 and ending at day 15; rates of apoptosis, growth and vascular development were compared at each time point (FIG. 16). A statistically significant difference in vessel development at day 9 was observed between vehicle and BAY 43-9006 treated tumors (FIG. 5A)(Student's t-test; P<0.05). In contrast, no statistically significant difference was detected in number of proliferating cells (Student's t-test; P=0.61) or apoptotic areas (Student's t-test; P=0.15) in tumor masses at day 9 between control and BAY 43-9006 treated tumors (FIG. 16B and FIG. 16C). However, for all analyses from day 11 onwards, a statistically significant difference was observed between control and drug treated tumors (Student's t-test; P<0.05). Collectively, these data suggest that significantly reduced vascular development observed at day 9 in BAY 43-9006 treated tumors was an initiating event leading to delayed tumor growth. Apoptosis became evident in the BAY 43-9006 treated tumors at day 11 and occupied up to 25% of the tumor area by day 15 (FIG. 16B). By day 20, ˜50% of the tumor area was undergoing apoptosis (data not shown). BAY 43-9006 also affected tumor cell proliferation of preexisting tumors leading to a 32-57% decrease in percentage of proliferating cells (FIG. 16C). Collectively, these data led to the conclusion that inhibition of vascular development is a key event leading to growth inhibition of preexisting melanoma tumors.

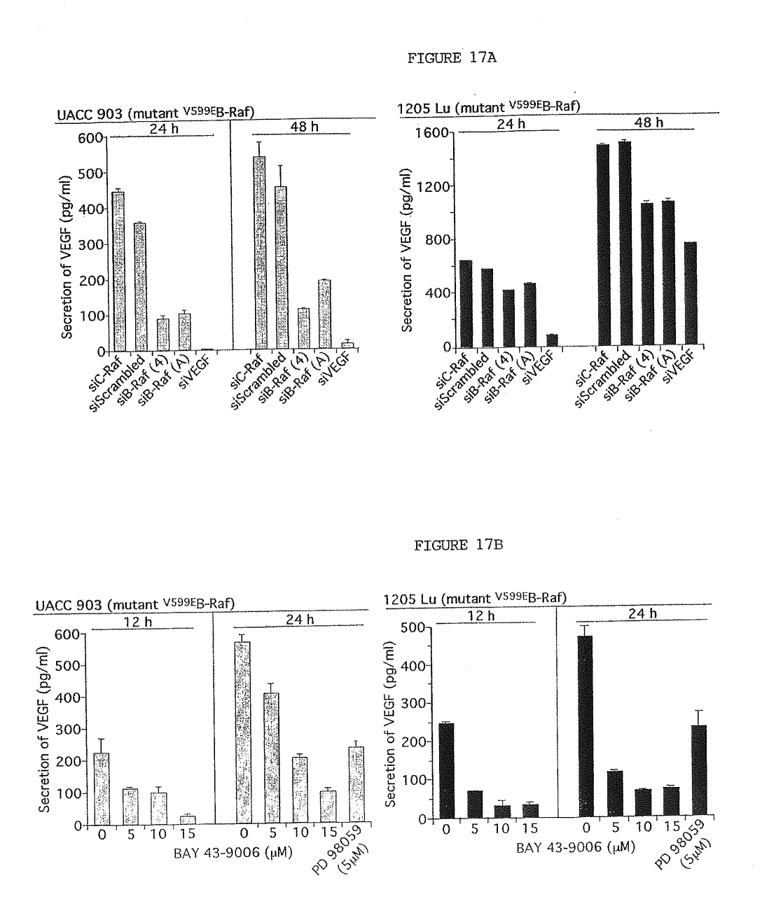

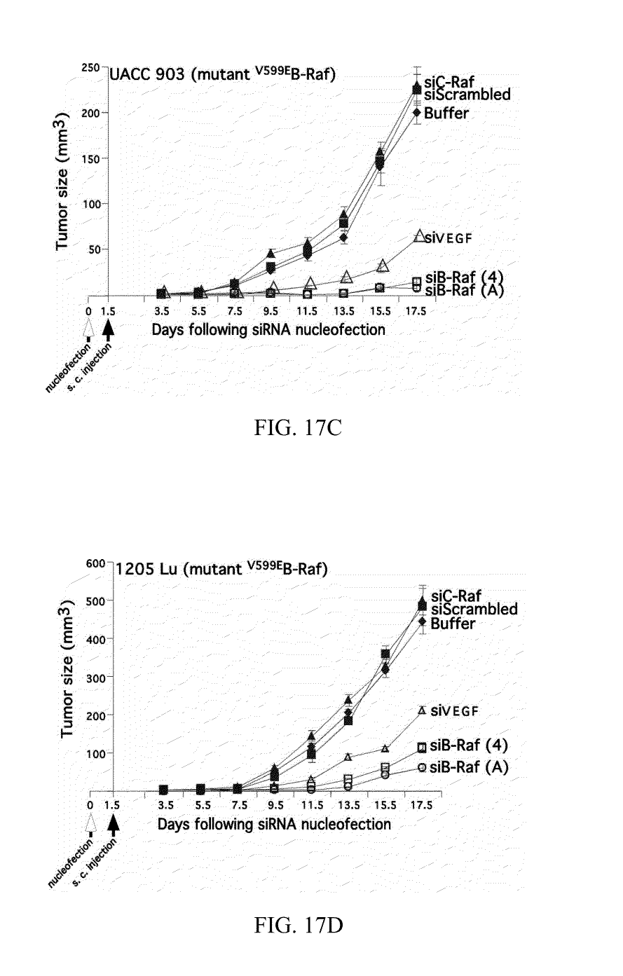

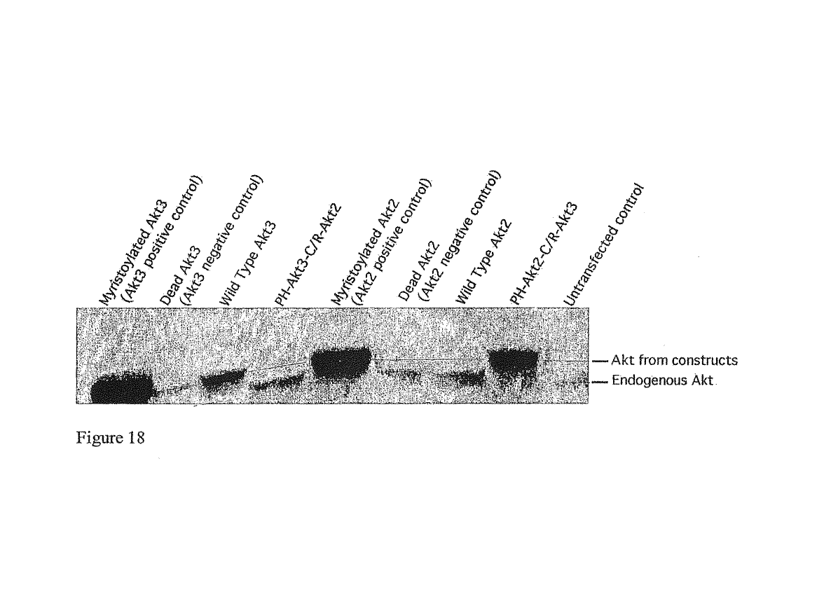

Since vascular development in tumors occurs via angiogenesis, or the growth of new vessels from the surrounding vascular beds, and is triggered by angiogenic factors secreted by tumor cells (Carmeliet P Jain R K. Nature. 407: 249-57 (2000).), we predicted that BAY 43-9006 and siRNA-mediated inhibition of ^{V599E}B-Raf were reducing the activity of a key angiogenic factor, thereby decreasing vascular development (Kranenburg O et al. Biochim Biophys Acta. 1654: 23-37 (2004)., Jain R K. Semin Oncol. 29: 3-9 (2002).). To examine this possibility, an ELISA assay was used to determine whether secretion of VEGF decreased following inhibition of ^{V599E}B-RAF. Initially, UACC 903 and 1205 Lu cells in which ^{V599E}B-Raf expression was inhibited using siRNA were examined and revealed significant reduction in VEGF secretion compared to controls (FIG. 17A). Next, the effects of BAY 43-9006 mediated inhibition of ^{V599E}B-Raf UACC 903 and 1205 Lu cells was examined and also found to decrease VEGF secretion in a dose dependent manner (FIG. 17B). To determine whether siRNA-mediated reduction of VEGF resulted in tumor inhibition similar to that seen following ^{V599E}B-Raf inhibition, siRNA against VEGF was nucleofected into UACC 903 or 1205 Lu cells. Decreased VEGF expression was observed using VEGF specific siRNA (FIG. 17A), which reduced the tumorigenic potential of UACC 903 (FIG. 17C) and 1205 Lu (FIG. 17D) cells in a manner consistent with that occurring following decreased ^{V599E}B-Raf expression. Thus, reduced VEGF secretion mediated by decreased ^{V599E}B-Raf activity led to inhibition of vascular development, which consequently affected melanoma tumor development.

### Experimental Discussion for B-Raf

This study demonstrates that use of siRNA or pharmacological inhibition of mutant ^{V599E}B-Raf expression (activity) effectively reduces the tumorigenic potential of melanoma cells by lowering the proliferative and/or angiogenic capacity of the tumor cell. As such, melanoma cells having mutant ^{V599E}B-Raf are better suited for proliferation in the in vivo tumor environment. We have shown that targeted reduction of ^{V599E}B-Raf expression (activity) in melanoma cells prior to tumor development significantly reduced the growth potential of melanoma cells, thereby inhibiting tumor development. In contrast, apoptosis played no significant role in this process. Furthermore, inhibition of tumor development was only observed in cells in which mutant ^{V599E}B-Raf expression had been knocked down and not following knockdown of C-Raf or following knockdown of B-Raf in melanoma cells lacking the T1796A B-RAF mutation. Therefore, it is apparent that signaling through ^{V599E}B-Raf was specifically necessary for melanoma tumor development. These data are consistent with our previous study demonstrating that siRNA-mediated inhibition of ^{V599E}B-Raf in WM793 melanoma cells reduced the in vitro growth potential of these cells (Hingorani S R et al. Cancer Res.; 63: 5198-202 (2003).). Similar in vitro studies using UACC 903 cells in this report further confirm these earlier observations. Knockdown of mutant ^{V599E}B-Raf expression (activity) also specifically reduced constitutive Erk signaling leading to reduced growth, which did not occur following knockdown of C-Raf. Thus, mutant ^{V599E}B-Raf promotes growth of melanoma cells both in vitro and in vivo; moreover, targeted inhibition prior to tumor development inhibits tumorigenesis mediated through reduced growth of tumor cells.

Targeting mutant ^{V599E}B-Raf in preexisting established tumors halted growth; however, growth inhibition played only a partial role in this process. More significantly, comparison of size and time matched tumors revealed that inhibition of vascular development played an initiating role in delaying tumor growth. As in all solid tumors, vascular development occurs through angiogenesis in which growth of new vessels from surrounding vascular beds is driven by angiogenic factors secreted by tumor cells (Carmeliet P, Jain R K. Nature. 407: 249-57 (2000).). In this study, we found that inhibition of ^{V599E}B. Raf reduced VEGF secretion by UACC 903 and 1205 Lu melanoma cells. B-Raf has been reported to exert an important role in embryonic vascular development since B-RAF knockout mice exhibit significant endothelial cell death leading to hemorrhage and embryonic lethality (Wojnowski L et al. Nat Genet; 16: 293-7 (1997).). However, we observed no significant endothelial cell death in preexisting tumor vessels following inhibition of ^{V599E}B-Raf using BAY 43-9006. Rather, inhibition of ^{V599E}B-Raf inhibited angiogenesis (Kranenburg O et al. BioChim Biophys Acta. 1654: 23-37 (2004)., Jain R K. Semin Oncol. 29: 3-9 (2002).) mediated through reduced VEGF secretion by the tumor cells. This observation is supported by published evidence in which decreased VEGF secretion led to reduced angiogenesis, thereby inhibiting the tumorigenic potential of cancer cells (Heidenreich R et al. Int J. Cancer. 111: 348-57 (2004)., Inai T et al. Am J Pathol. 165: 35-52 (2004).). Thus, decreased VEGF secretion mediated by a reduction in mutant ^{V599E}B-Raf signaling leads to inhibition of angiogenesis, halting growth of preexisting melanoma tumors.

Our study also shows that BAY 43-9006 inhibits ^{V599E}B-Raf activity in vitro and in vivo, leading to reduced phosphorylation of downstream targets Mek and Erk, which slowed melanoma tumor development. We observed that pretreatment of animals with BAY 43-9006 reduced melanoma tumor development in manner similar to siRNA-mediated inhibition. However, BAY 43-9006 treatment only retarded development of established tumors by disrupting their vascular development. Complete regression of tumors did not occur, rather tumor size became relatively static after treatment. This observation is in agreement with preliminary data from clinical trials in which BAY 43-9006 monotherapy was relatively ineffective for treatment of advanced stage melanoma patients (Tuveson D A et al. Cancer Cell. 4: 95-8 (2003)., Ahmad T et al. Proc Am Soc Clin Oncol. 23: 708 (2004).). However, in combination with traditional chemotherapy (paclitaxel and carboplatinum), a 50% response rate occurred in patients (Tuveson D A et al. Cancer Cell. 4: 95-8 (2003)., Flaherty K et al. Proc Am Soc Clin Oncol. 23: 708 (2004).). Therefore, while BAY 43-9006 slows tumor development, it is likely that the drug will need to be combined with other synergistic therapeutics to cause regression of established preexisting tumors (Tuveson D A et al. Cancer Cell. 4: 95-8 (2003)., Bollag G et al. Curr Opin Investig Drugs. 4: 1436-41 (2003)., Lyons J F et al. Endocr Relat Cancer. 8: 219-25 (2001).). It is also possible that the route of drug administration could alter efficacy of BAY 43-9006 in melanoma patients. While the clinical trial involved oral administration of the drug, our study administered the drug via intra peritoneal injection every 2 to 3 days. An alternative route of administration might be more effective by increasing the drug's local bioavailability (Sparreboom A et al. Proc Natl Acad Sci USA. 94: 2031-5 (1997)., Bardelmeijer H A et al. Cancer Research. 62: 6158-64 (2002)., Hale J T et al. Bioch Pharm. 64: 1493-502 (2002)., Kimura. Y et al. Cancer Chemother Pharm. 49: 322-8 (2002).). Therefore, therapeutically targeting ^{V599E}B-Raf activity in combination with chemotherapeutic agents may offer an effective approach to shrink established melanoma tumors containing this mutant protein.

In conclusion, we identified mechanisms by which mutant V599E B-Raf promotes melanoma tumor development and show how this mutation provides melanoma cells with selective growth and angiogenic advantages in the tumor environment.

### Example 24

### Akt3 Domain Swap Experiments, Results and Discussion

Domain switching between the Akt isoforms has identified the region of Akt3 leading to preferential activation of Akt3 and not Akt1 or Akt2 in melanoma. Activation is measured as the levels of phosphorylation of threonine 308 or serine 472 on Akt3; or by immunoprecipitation of Akt3 followed by an in vitro kinase assay in which Crosstide is phosphorylated by Akt3 to estimate activity. Domains of Akt3 were switched with those of Akt2 or Akt1 and constructs containing the chimeric genes were nucleofected into the melanoma cell lines WM35 or UACC 903. Myristoylated Akt3 and Akt2 served as positive control while dead Akt3 (T305A/S472A) and Akt2 (T309A/S474A) served as negative controls. Transfer of wild type Akt3 led to increased activity in contrast to wild type Akt2 that did not, which demonstrated that specificity for Akt3 activation in melanoma cells. Constructs in which the pleckstrin homology domain from Akt3 (amino acids 1-110) was connected to the catalytic-regulatory domains of Akt2 did not lead to activation. In contrast, constructs in which the pleckstrin homology domain from Akt2 (amino acids 1-110) was connected to the catalytic-regulatory domains from Akt3 (from amino acids 111-497) were activated. This maps the critical region leading to preferential Akt3 activation in melanomas from amino acids 111-497. This is the region to which therapeutic agents may be targeted to specifically prevent Akt3 activation in melanomas.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention. All documents (e.g., publications and patent applications) cited herein are incorporated by reference to the same extent as if each individual document was specifically and individually indicated to be incorporated by reference.

### REFERENCES

Ahmad T, Marais R, Pyle L, al. e. BAY 43-9006 in patients with advanced melanoma: The Royal Marsden experience. Proc Am Soc Clin Oncol 2004; 23:708.
Alessi D R, Andjelkovic M, Caudwell B, Cron P, Morrice N, Cohen P, Hemmings B A. Mechanism of activation of protein kinase B by insulin and IGF-1. EMBO J. 15(23):6541-51 (1996).
Andjelkovic M, Jones P F, Grossniklaus U, Cron P, Schier A F, Dick M, Bilbe G, Hemmings B A. Developmental regulation of expression and activity of multiple forms of the Drosophila RAC protein kinase. J Biol. Chem. 270(8):4066-75 (1995).
Atkins, J. H., and Gershell, L. J., Selective anticancer drugs, Nature Rev. Drug Dis 1:491-492 (2002).
Ballo, M. T., and Ang, K. K., Radiation therapy for malignant melanoma, Surgical Clinics North Am 83:323-342 (2003).
Bankston D, Dumas J, Natero R, Riedl B, Monahan M-K, Sibley R. A Scaleable Synthesis of BAY 43-9006: A Potent Raf Kinase Inhibitor for the Treatment of Cancer. Organic Process Res Dev 2002; 6:777-81.
Bardelmeijer H A, Ouwehand M, Buckle T, et al. Low systemic exposure of oral docetaxel in mice resulting from extensive first-pass metabolism is boosted by ritonavir. Cancer Research 2002; 62:6158-64.
Bastian, B. C., LeBoit, P. E., Hamm, H., Brocker, E. B., and Pinkel, D., Chromosomal gains and losses in primary cutaneous melanomas detected by comparative genomic hybridization, Cancer Res 58:2170-2175 (1998).
Bellacosa, A., de Feo, D., Godwin, A. K., Bell, D. W., Cheng, J. Q., Altomare, D. A., Wan, M., Dubeau, L., Scambia, G., and Masciullo, V., Molecular alterations of the AKT2 oncogene in ovarian and breast carcinomas, Int J Cancer 64:280-285 (1995).
Bellacosa A, Testa J R, Moore R, Larue L. A portrait of AKT kinases: human cancer and animal models depict a family with strong individualities. Cancer Biol Ther. 3(3):268-75. Epub 2004 (2004).
Bollag G, Freeman S, Lyons J F, Post L E. Raf pathway inhibitors in oncology. Curr Opin Investig Drugs 2003; 4:1436-41.
Brazil D P, Hemmings B A. Ten years of protein kinase B signalling: a hard Akt to follow. Trends Biochem Sci. 26(11):657-64 (2001).
Brazil, D. P., Park, J., and Hemmings, B. A., PKB binding proteins. Getting in on the Akt, Cell 111:293-303 (2002).
Brazil D P, Yang Z Z, Hemmings B A. Advances in protein kinase B signalling: AKTion on multiple fronts. Trends Biochem Sci. 29(5):233-42 (2004).
Brodbeck D., Cron, P., and Hemmings, B. A., A human protein kinase Bgamma with regulatory phosphorylation sites in the activation loop and in the C-terminal hydrophobic domain, J Biol Chem 274:9133-9136 (1999).
Brodbeck D, Hill M M, Hemmings B A. Two splice variants of protein kinase B gamma have different regulatory capacity depending on the presence or absence of the regulatory phosphorylation site serine 472 in the carboxyl-terminal hydrophobic domain. J Biol Chem. 276(31):29550-8. Epub 2001 (2001).
Brose M S, Volpe P, Feldman M, et al. BRAF and RAS mutations in human lung cancer and melanoma. Cancer Res 2002; 62:6997-7000.
Carmeliet P, Jain R K. Angiogenesis in cancer and other diseases. Nature 2000; 407:249-57.
Chang F, Steelman L S, Shelton J G, et al. Regulation of cell cycle progression and apoptosis by the Ras/Raf/MEK/ERK pathway. Int J Oncol 2003; 22:469-80.
Chen, W. S., Xu, P. Z., Gottlob, K., Chen, M. L., Sokol, K., Shiyanova, T., Roninson, I., Weng, W., Suzuki, R., To be, K., et al., Growth retardation and increased apoptosis in mice with homozygous disruption of the Akt1 gene, Genes Dev 15:2003-2208 (2001).
Cheng, J. Q., Godwin, A. K., Bellacosa, A., Taguchi, T., Franke, T. F., Hamilton, T. C., Tsichlis, P. N., and Testa, J. R., AKT2 a putative oncogene encoding a member of a subfamily of protein-serine/threonine kinase, is amplified in human ovarian carcinomas, Proc Nat Acad Sciences USA 89:9267-9271 (1992).
Cheng, J. Q., Ruggeri, B., Klein, W. M., Sonoda, G., Altomare, D. A., Watson, D. K., and Testa, J. R., Amplification of AKT2 in human pancreatic cells and inhibition of AKT2 expression and tumorigenicity by antisense RNA, Proc Nat Acad Sciences USA 93:3636-3641 (1996).
Cho, H., Mu, J., Kim, J. K., Thorvaldsen, J. L., Chu, Q., Crenshaw, E. B., 3rd, Kaestner, K. H., Bartolomei, M. S., Shulman, G. I., and Birnbaum, M. J., Insulin resistance and a diabetes mellitus-like syndrome in mice lacking the protein kinase Akt2 (PKB beta), Science 292:1728-1731 (2001).
Cho, H., Thorvaldsen, J. L., Chu, Q., Fent, F., and Birnbaum, M. J., Akt1/PKBalpha is required for normal growth but dispensable for maintenance of glucose homeostasis in mice, J Biol Chem 276-38349-38352 (2001).
Chong H, Vikis H G, Guan K L. Mechanisms of regulating the Raf kinase family. Cell Signal 2003; 15:463-9.
Datta S R, Brunet A, Greenberg M E. Cellular survival: a play in three Akts. Genes Dev. 13(22):2905-27 (1999).
Davies H, Bignell G R, Cox C, et al. Mutations of the BRAF gene in human cancer. Nature 2002; 417:949-54.
Dhawan, P., Singh, A. B., Ellis, D. O L., and Richmond, A., Constitutive activation of Akt/protein kinase B in melanoma leads to up-regulation of nuclear factor-kappaB and tumor progression, Cancer Res 62:7335-7342 (2002).
Ferguson K M, Kavran J M, Sankaran V G, Fournier E, Isakoff S J, Skolnik E Y, Lemmon M A. Structural basis for discrimination of 3-phosphoinositides by pleckstrin homology domains. Mol. Cell. 6(2):373-84 (2000).
Flaherty K, Brose M S, Schuchter L, et al. Phase I/II trial of BAY 43-9006, carboplatin and paclitaxel demonstrates preliminary antitumor activity in the expansion cohort of patients with metastatic melanoma. Proc Am Soc Clin Oncol 2004; 23:708.
Gorden A, Osman I, Gai W, et al. Analysis of BRAF and N-RAS mutations in metastatic melanoma tissues. Cancer Res 2003; 63:3955-7.
Grossman, D., and Altieri, D. C., Drug resistance in melanoma: mechanisms, apoptosis, and new potential therapeutic targets, Cancer Metastasis Rev 20:3-11 (2001).
Hale J T, Bigelow J C, Mathews L A, McCormack J J. Analytical and pharmacokinetic studies with 5-chloro-2′-deoxycytidine. Bioch Pharm 2002; 64:1493-502.
Heidenreich R, Machein M, Nicolaus A, et al. Inhibition of solid tumor growth by gene transfer of VEGF receptor-1 mutants. Int J Cancer 2004; 111:348-57.
Helmbach, H., Rossmann, E., Kern, M. A., and Schadendorf, D., Drug-resistance in human melanoma, Int J. Cancer 93:617-622 (2001).
Herlyn, M., Molecular and cellular biology of melanoma: Austin: R.G. Landes Co. (1993).
Hersey, P., Adjuvant therapy for high-risk primary and resected metastatic melanoma, Int Med J 33:33-43 (2003).
Hingorani S R, Jacobetz M A, Robertson G P, Herlyn M, Tuveson D A. Suppression of BRAF(V599E) in human melanoma abrogates transformation. Cancer Res 2003; 63:5198-202.
Hsu, M.-Y., Elder, D. E., and Herlyn, M., Melanoma: The Wistar (WM) Cell Lines. In Human Cell Culture. J.R.W.M.a.B. Palsson, editor. Great Britain: Kluwer Academic Publishers, 259-274 (1999).
Inai T, Mancuso M, Hashizume H, et al. Inhibition of vascular endothelial growth factor (VEGF) signaling in cancer causes loss of endothelial fenestrations, regression of tumor vessels, and appearance of basement membrane ghosts. Am J Pathol 2004; 165:35-52.
Jain R K. Tumor angiogenesis and accessibility: role of vascular endothelial growth factor. Semin Oncol 2002; 29:3-9.
Jemal, A., Devesa, S. S. Hartge, P., and Tucker, M. A., Recent trends in cutaneous melanoma incidence among whites in the United States., J Nat Cancer Inst 93:678-683 (2001).
Jemal, A., Thomas, A., Murray, T., and Thun, M., Cancer statistics, 2002. Ca: a Cancer Journal for Clinicians 52:23-47 (2002).
Johnstone, R. W., Ruefli, A. A., and Lowe, S. W., Apoptosis: a link between cancer genetics and chemotherapy, Cell 108:153-164 (2002).
Jones P F, Jakubowicz T, Hemmings B A. Molecular cloning of a second form of rac protein kinase. Cell Regul. 2(12):1001-9 (1991).
Kimura Y, Aoki J, Kohno M, Ooka H, Tsuruo T, Nakanishi O. P-glycoprotein inhibition by the multidrug resistance-reversing agent MS-209 enhances bioavailability and antitumor efficacy of orally administered paclitaxel. Cancer Chemother Pharm 2002; 49:322-8.
Kranenburg O, Gebbink M F, Voest E E. Stimulation of angiogenesis by Ras proteins. Biochim Biophys Acta 2004; 1654:23-37.
Krasilnikov, M., Adler, V., Fuchs, S. Y., Dong, Z., Haimovitz-Friedman, A., Herlyn, M., and Ronai, Z., Contribution of phosphatidylinositol 3-kinase to radiation resistance in human melanoma cells, Mol Carcinogenesis 24:64-69 (1999).
Kumar et al., BRAF Mutations in Metastatic Melanoma, Clinical Cancer Research, 9: 3362-3368, (2003).
Laine, J., Kunstle, G., Obata, T., and Noguchi, M., Differential regulation of Akt kinase isoforms by the members of the TCL1 oncogene family, J Biol Chem 277:3743-3751 (2002).
Lang J, Boxer M, MacKie R. Absence of exon 15 BRAF germline mutations in familial melanoma. Hum Mutat 2003; 21:327-30.
Laud K, Kannengiesser C, Avril M F, et al. BRAF as a melanoma susceptibility candidate gene? Cancer Res 2003; 63:3061-5.
Lietzke S E, Bose S, Cronin T, Klarlund J, Chawla A, Czech M P, Lambright D G. Structural basis of 3-phosphoinositide recognition by pleckstrin homology domains. Mol. Cell. 6(2):385-94 (2000).
Lowinger T B, Riedl B, Dumas J, Smith R A. Design and discovery of small molecules targeting raf-1 kinase. Curr Pharm Des 2002; 8:2269-78.
Lu, Y., Li, Z., and Sun, M., Multiple gene alterations involved in the processor of human gastric carcinogenesis, Chung-Hua I Hsuch Tsa Chih [Chinese Medical Journal] 75:679-682 (1995).
Luo Y, Smith R A, Guan R, Liu X, Klinghofer V, Shen J, Hutchins C, Richardson P, Holzman T, Rosenberg S H, Giranda V L. Pseudosubstrate peptides inhibit Akt and induce cell growth inhibition. Biochemistry. 43(5):1254-63 (2004).
Lyons J F, Wilhelm S, Hibner B, Bollag G. Discovery of a novel Raf kinase inhibitor. Endocr Relat Cancer 2001; 8:219-25.
Mercer K E, Pritchard C A. Raf proteins and cancer: B-Raf is identified as a mutational target. Biochim Biophys Acta 2003; 1653:25-40.
Mertens, F., Johansson, B., Höglund, M., and Mitelman, F., Chromosomal imbalance maps of malignant solid tumors: a cytogenetic survey of 3185 neoplasms, Cancer Res 57:2765-2780 (1997).
Meyer P, Klaes R, Schmitt C, Boettger M B, Garbe C. Exclusion of BRAFV599E as a melanoma susceptibility mutation. Int J Cancer 2003; 106:78-80.
Miller C J, Cheung M, Sharma A, et al. Method of Mutation Analysis May Contribute to Discrepancies in Reports of BRAF Mutation Frequencies in Melanocytic Neoplasms. J Invest Dermatol 2004; 123:990-2.
Mitsuuchi, Y., Johnson, S. W., Moonblatt, S., and Testa, J. R., Translocation and activation of AKT2 in response to stimulation by insulin, J Cellular Biochem 70:433-441 (1998).
Nicholson, K. M., and Anderson, N. G., The protein kinase B/Akt signaling pathway in human malignancy, Cell Signal 14:381-395 (2002).
Niv M Y, Rubin H, Cohen J, Tsirulnikov L, Licht T, Peretzman-Shemer A, Cna'an E, Tartakovsky A, Stein I, Albeck S, Weinstein I, Goldenberg-Furmanov M, Tobi D, Cohen E, Laster M, Ben-Sasson S A, Reuveni H. Sequence-based design of kinase inhibitors applicable for therapeutics and target identification. J Biol. Chem. 279(2):1242-55. Epub 2003 (2004).
Obata T, Yaffe M B, Leparc G G, Piro E T, Maegawa H, Kashiwagi A, Kikkawa R, Cantley L C. Peptide and protein library screening defines optimal substrate motifs for AKT/PKB. J Biol. Chem. 275(46):36108-15 (2000).
Parmiter, A. H., Balaban, G., Clark, W. H., Jr., and Nowell, P. C., Possible involvement of the chromosome region 10q24---q26 in early stages of melanocytic neoplasia, cancer Genet Cytogenet 30:313-317 (1988).
Peng, X. D., Xu, P. Z., Chen, M. L., Hahn-Windgassen, A., Skeen, J., Jacobs, J., Sundararajan, D., Chen, W. S., Crawford, S. E., Coleman, K. G., et al., Dwarfism, impaired skin development, skeletal muscle atrophy, delayed bone development, and impeded adipogenesis in mice lacking Akt1 and Akt2, Genes Dev. 17:1352-1365 (2003).
Pollock P M, Harper U L, Hansen K S, et al. High frequency of BRAF mutations in nevi. Nat Genet. 2003; 33:19-20.
Powell, D. J., Hajduch, E., Kular, G., and Hundal, H. S., Ceramide Disables 3-Phosphoinoside Binding to the Pleckstrin Homology Domain of Protein Kinase B (PKB)/Akt by a PKCzeta-Dependent Mechanism, Mol Cell Biol 23:7794-7808 (2003).
Robertson, G. P., Herbst, R. A., Nagane, M., Huang, H. J., and Cavenee, W. K., The chromosome 10 monosomy common in human melanomas results from loss of two separate tumor suppressor loci, Cancer Res 59:3596-3601 (1999).
Robertson G P, Furnari F B, Miele M E, et al. In vitro loss of heterozygosity targets the PTEN/MMAC1 gene in melanoma. Proc Natl Acad Sci USA 1998; 95:9418-23.
Sasaki Y, Niu C, Makino R, et al. BRAF Point Mutations in Primary Melanoma Show Different Prevalences by Subtype. J Invest Dermatol 2004; 123:177-83.
Satyamoorthy K, Li G, Gerrero M R, et al. Constitutive mitogen-activated protein kinase activation in melanoma is mediated by both BRAF mutations and autocrine growth factor stimulation. Cancer Res 2003; 63:756-9.
Scheid M P, Woodgett J R. PKB/AKT: functional insights from genetic models. Nat Rev Mol Cell Biol. 2(10):760-8 (2001).
Scheid M P, Woodgett J R. Unravelling the activation mechanisms of protein kinase B/Akt. FEBS Lett. 546(1):108-12 (2003).
Schalick, W. O., Albino, A. P., Reed, J. A., Fountain, J. W., Scott, G. A., SuPont, G., Puri, N., Herlyn, M., Halern, A. C., Gallager, R. P., et al., Cutaneous Oncology. Malden, M A: Blackwell Science, Inc. 180-340 (1998).
Serrone, L., and Herseym, P., The chemoresistance of human malignant melanoma: an update, Melanoma Res 9:51-58 (1999).
Serrone, L., Zeuli, M., Sega, F. M., and Cognetti, F., Dacarbazine-based chemotherapy for metastatic melanoma: thirty-year experience overview, J Exp Clin Cancer Res 19:21-34 (2000).
Smalley K S. A pivotal role for ERK in the oncogenic behaviour of malignant melanoma? Int J Cancer 2003; 104:527-32.
Soengas, M. S., and Lowe, S. W., Apoptosis and melanoma chemoresistance, Oncogene 22:3138-3151 (2003).
Staal, S. P., Molecular cloning of the akt oncogene and its human homologues AKT1 and AKT2: amplification of AKT1 in a primary human gastric acenocarcinoma, Proc Nat Acad Sciences USA 84:5034-5037 (1987).
Stahl, J. M., Cheung, M., Sharma, A., Trivedi, N. R., Shanmugam, S., and Robertson, G. P., Loss of PTEN Promotes Tumor Development in Malignant Melanoma, Cancer Res 63:2891-2897 (2003).
Sparreboom A, van Asperen J, Mayer U, et al. Limited oral bioavailability and active epithelial excretion of paclitaxel (Taxol) caused by P-glycoprotein in the intestine. Proceedings of the National Academy of Sciences of the United States of America 1997; 94:2031-5.
Stahl J M, Sharma A, Cheung M, et al. Deregulated Akt3 activity promotes development of malignant melanoma. Cancer Res 2004; 64:7002-10.
Sumimoto H, Miyagishi M, Miyoshi H, et al. Inhibition of growth and invasive ability of melanoma by inactivation of mutated BRAF with lentivirus-mediated RNA interference. Oncogene 2004.
Sun, M., Wang, G., Paciga, J. E., Feldman, R. I., Yuan, Z. Q., Ma, X. L., Shelley, S. A., Jove, R., Tsichlis, P. N., Nicosia, S. V., et al., AKT1/PKBalpha kinase is frequently elevated in human cancers and its constitutive activation is required for oncogenic transformation in NIH3T3 cells, Am J Path 159:431-437 (2001).
Testa J R, Bellacosa A. AKT plays a central role in tumorigenesis. Proc Natl Acad Sci U S A. 98(20):10983-5 (2001).
Tsao H, Goel V, Wu H, Yang G, Haluska F G. Genetic interaction between NRAS and BRAF mutations and PTEN/MMAC 1 inactivation in melanoma. J Invest Dermatol 2004; 122:337-41.
Tuveson D A, Weber B L, Herlyn M. BRAF as a potential therapeutic target in melanoma and other malignancies. Cancer Cell 2003; 4:95-8.
Thompson, F. H., Emerson, J., Olson, S., Weinstein, R., Leavitt, S. A., Leong, S. P., Emerson, S., Trent, J. M., Nelson, M. A., Salmon, S. E., et al., Cytogenetics of 158 patients with regional or disseminated melanoma. Subset analysis of near-diploid and simple karyotypes, Cancer Genet Cytogenet 83:93-104 (1995).
Uribe P, Wistuba, I I, Gonzalez S. BRAF mutation: a frequent event in benign, atypical, and malignant melanocytic lesions of the skin. Am J Dermatopathol 2003; 25:365-70.
van Dekken, H., Geelen, E., Dinjens, W. N., Wijnhoven, B. P., Tilanus, H. W., Tanke, H. J., and Rosenberg, C., Comparative genomic hybridization of cancer of the gastroesophageal junction: deletion of 14Q31-32.1 discriminates between esophageal (Barrett's) and gastric cardia adenocarcinomas, Cancer Res 59:748-752 (1999).
Waldmann, V., Wacher, J., and Deichmann, M., Absence of mutations in the pleckstrin homology (PH) domain of protein kinase B (PKB/Akt) in malignant melanoma, Melanoma Res 12:45-50 (2002).
Waldmann, V., Wacker, J., and Diechmann, M., Mutations of the activation-associated phosphorylation sites at codons 308 and 473 of protein kinase B are absent in human melanoma, Arch Dermatol Res 293:368-372 (2001).
Wojnowski L, Zimmer A M, Beck T W, et al. Endothelial apoptosis in Braf-deficient mice. Nat Genet. 1997; 16:293-7.
Yazdi A S, Palmedo G, Flaig M J, et al. Mutations of the BRAF gene in benign and malignant melanocytic lesions. J Invest Dermatol 2003; 121:1160-2.

What is claimed is:

1. A method for inducing apoptosis in a melanoma tumor cell comprising:
reducing Akt3 activity.

2. The method of claim 1 wherein said reducing is by contacting a melanoma tumor cell with an agent that reduces Akt3 activity.

3. The method of claim 2 wherein the agent is selected from the group consisting of a siRNA molecule, an antisense molecule, an antagonist, a ribozyme, an inhibitor, a peptide, and a small molecule.

4. The method of claim 3 wherein the agent is a siRNA molecule that comprises a polynucleotide selected from the group having a sequence of 5′ GGUCUAGCUACAGAGAAAUCUCGAU 3′ (SEQ ID NO:10), 5′ CUAUCUACAUUCCGGAAAG 3′ (SEQ ID NO:1), 5′ GAAUUUACAGCUCAGACUA 3′ (SEQ ID NO:2), 5′ CAGCUCAGACUAUUACAAU 3′ (SEQ ID NO:3), 5′CUUGGACUAUCUACAUUCCGGAAAG 3′ (SEQ ID NO:4), 5′CUUUCCGGAAUGUAGAUAGUCCAAG 3′ (SEQ ID NO:5), 5′GAUGAAGAAUUUACAGCUCAGACUA 3′ (SEQ ID NO:6), 5′UAGUCUGAGCUGUAAAUUCUUCAUC 3′ (SEQ ID NO:7), 5′AAUUUACAGCUCAGACUAUUACAAU 3′ (SEQ ID NO:8), 5′AUUGUAAUAGUCUGAGCUGUAAAUU 3′ (SEQ ID NO:9), and the complements thereof.

5. The method of claim 2 wherein said contacting of said melanoma tumor cell includes the use of: a liposome, a nanoliposome, a ceramide-containing nanoliposome, a proteoliposome, a nanoparticulate, a calcium phospho-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, calcium phosphate nucleotide-mediated nucleotide delivery, electroporation, and microinjection.

6. The method of claim 3 wherein said agent is a peptide that acts as a pseudosubstrate for Akt3.

7. The method of claim 6 wherein said peptide acts as a pseudosubstrate for a catalytic domain or a regulatory domain of Akt3.

8. The method of claim 3 wherein said agent is a peptide that acts as a competitive inhibitor for Akt3.

9. The method of claim 8 wherein said peptide acts as a competitive inhibitor for a catalytic domain of Akt3.

10. The method of claim 8 wherein said peptide acts as a competitive inhibitor for a pleckstrin homology domain of Akt3.

11. The method of claim 8 wherein said peptide acts as a competitive inhibitor for a regulatory domain of Akt3.

12. The method of claim 1 wherein the method further comprises: administering a chemotherapeutic agent selected from the group consisting of alkylating agents, antimetabolites, antibiotics, natural or plant derived products, hormones and steroids, and platinum drugs.

13. The method of claim 12 wherein the chemotherapeutic agent is dacarbazine.

14. The method of claim 1 wherein the method further comprises administering irradiation.

15. A method for treating a melanoma tumor in a mammal comprising:
administering to a melanoma tumor an effective amount of an agent to induce apoptosis; and administering to a melanoma tumor an effective amount of an agent to reduce angiogenesis and cell proliferation.

16. The method of claim 15 wherein said agent that induces apoptosis is an agent that reduces Akt3 activity.

17. The method of claim 15 wherein said agent that reduces angiogenesis and cell proliferation is an agent that reduces V599E B-Raf activity, thereby treating a melanoma tumor.

18. The method of claim 16 wherein said agent that reduces Akt3 activity is selected from the group consisting of a siRNA molecule, an antisense molecule, an antagonist, a ribozyme, an inhibitor, a peptide, and a small molecule.

19. The method of claim 18 wherein said agent that reduces Akt3 activity is a siRNA molecule that comprises a polynucleotide selected from the group having a sequence of 5′ GGUCUAGCUACAGAGAAAUCUCGAU 3′ (SEQ ID NO:10), 5′ CUAUCUACAUUCCGGAAAG 3′ (SEQ ID NO:1), 5′ GAAUUUACAGCUCAGACUA 3′ (SEQ ID NO:2), 5′ CAGCUCAGACUAUUACAAU 3′ (SEQ ID NO:3), 5′CUUGGACUAUCUACAUUCCGGAAAG 3′ (SEQ ID NO:4), 5′ CUUUCCGGAAUGUAGAUAGUCCAAG 3′ (SEQ ID NO:5), 5′GAUGAAGAAUUUACAGCUCAGACUA (SEQ ID NO:6), 5′UAGUCUGAGCUGUAAAUUCUUCAUC 3′ (SEQ ID NO:7), 5′AAUUUACAGCUCAGACUAUUACAAU 3′ (SEQ ID NO:8), 5′AUUGUAAUAGUCUGAGCUGUAAAUU 3′ (SEQ ID NO:9), and the complements thereof.

20. The method of claim 16 wherein the agent that reduces Akt3 activity is introduced into said melanoma tumor by the use of: a liposome, a nanoliposome, a ceramide-containing nanoliposome, a proteoliposome, a nanoparticulate, a calcium phospho-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, calcium phosphate nucleotide-mediated nucleotide delivery, electroporation, and microinjection.

21. The method of claim 18 wherein said agent is a peptide that acts as a pseudosubstrate for Akt3.

22. The method of claim 21 wherein said peptide acts as a pseudosubstrate for a catalytic domain or a regulatory domain of Akt3.

23. The method of claim 18 wherein said agent is a peptide that acts as a competitive inhibitor for Akt3.

24. The method of claim 23 wherein said peptide acts as a competitive inhibitor for a catalytic domain of Akt3.

25. The method of claim 23 wherein said peptide acts as a competitive inhibitor for a pleckstrin homology domain of Akt3.

26. The method of claim 23 wherein said peptide acts as a competitive inhibitor for a regulatory domain of Akt3.

27. The method of claim 15 wherein the method further comprises administering a chemotherapeutic agent selected from the group consisting of alkylating agents, antimetabolites, antibiotics, natural or plant derived products, hormones and steroids, and platinum drugs.

28. The method of claim 15 wherein the method further comprises administering irradiation.

29. The method of claim 17 wherein the agent that reduces V599E B-Raf activity is selected from the group consisting of a siRNA molecule, an antisense molecule, an antagonist, a ribozyme, an inhibitor, a peptide, and a small molecule.

30. The method of claim 17 wherein the agent that reduces V599E B-Raf activity is introduced into said melanoma tumor by the use of: a liposome, a nanoliposome, a ceramide-containing nanoliposome, a proteoliposome, a nanoparticulate, a calcium phospho-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, calcium phosphate nucleotide-mediated nucleotide delivery, electroporation, and microinjection.

31. The method of 29 wherein the siRNA molecule that reduces V599E B-Raf activity comprises:
a polynucleotide that has a sequence of 5′ GGUCUAGCUACAGAGAAAUCUCGAU 3′ (SEQ ID NO:10).

32. The method of claim 29 wherein the siRNA molecule that reduces B-Raf activity comprises:
a polynucleotide that has a sequence of 5′ GGACAAAGAAUUGGAUCUGGAUCAU 3′ (SEQ ID NO:11).

33. The method of claim 29 wherein the agent that reduces V599E B-Raf activity is a B-Raf inhibitor.

34. The method of claim 33 wherein the B-Raf inhibitor is BAY 43-9006.

35. The method of claim 15, where in said treatment comprises:
administering, concurrently or sequentially, an effective amount of an agent that reduces Akt3 activity and an agent that reduces V599E B-Raf activity.

36. A pharmaceutical composition for treating a melanoma tumor comprising:
an agent that reduces Akt3 activity; and a carrier.

37. The pharmaceutical composition of claim 36 wherein said carrier is selected from a group consisting of: a liposome, a nanoliposome, a ceramide-containing nanoliposome, a proteoliposome, a nanoparticulate, a calcium phospho-silicate nanoparticulate, a calcium phosphate nanoparticulate, a silicon dioxide nanoparticulate, a nanocrystaline particulate, a semiconductor nanoparticulate, poly(Darginine), a nanodendrimer, a virus, and calcium phosphate nucleotide-mediated nucleotide delivery.

38. The pharmaceutical composition of claim 36 wherein said agent is selected from the group consisting of:
siRNA molecule, an antisense molecule, an antagonist, a ribozyme, an inhibitor, a peptide, and a small molecule.

39. The pharmaceutical composition of claim 38 wherein said small interfering RNA (siRNA) molecule comprises:
a polynucleotide 5′ GGUCUAGCUACAGAGAAAUCUCGAU 3′ (SEQ ID NO:10) or the complement thereof.

40. The pharmaceutical composition of claim 38 wherein said small interfering RNA (siRNA) molecule comprises:
5′ CUAUCUACAUUCCGGAAAG 3′ (SEQ ID NO:1), or the complement thereof.

41. The pharmaceutical composition of claim 38 wherein said small interfering RNA (siRNA) molecule comprises:
a polynucleotide 5′ GAAUUUACAGCUCAGACUA 3′ (SEQ ID NO:2), or the complement thereof.

42. The pharmaceutical composition of claim 38 wherein said small interfering RNA (siRNA) molecule comprises:
the polynucleotide 5′ CAGCUCAGACUAUUACAAU 3′ (SEQ ID NO:3), or the complement thereof.

43. The pharmaceutical composition of claim 38 wherein said small interfering RNA (siRNA) molecule comprises:
a polynucleotide 5′ CUUGGACUAUCUACAUUCCGGAAAG 3′ (SEQ ID NO:4), or the complement thereof.

44. The pharmaceutical composition of claim 38 wherein said small interfering RNA (siRNA) molecule comprises:
a polynucleotide 5′ CUUUCCGGAAUGUAGAUAGUCCAAG 3′ (SEQ ID NO:5), or the complement thereof.

45. The pharmaceutical composition of claim 38 wherein said small interfering RNA (siRNA) molecule comprises:
a polynucleotide 5′ GAUGAAGAAUUUACAGCUCAGACUA 3′ (SEQ ID NO:6), or the complement thereof.

46. The pharmaceutical composition of claim 38 wherein said small interfering RNA (siRNA) molecule comprises:
a polynucleotide 5′ UAGUCUGAGCUGUAAAUUCUUCAUC 3′ (SEQ ID NO:7), or the complement thereof.

47. The pharmaceutical composition of claim 38 wherein said small interfering RNA (siRNA) molecule comprises:
a polynucleotide 5′ AAUUUACAGCUCAGACUAUUACAAU 3′ (SEQ ID NO:8), or the complement thereof.

48. The pharmaceutical composition of claim 38 wherein said small interfering RNA (siRNA) molecule comprises:
a polynucleotide 5′ AUUGUAAUAGUCUGAGCUGUAAAUU 3′ (SEQ ID NO:9), or the complement thereof.

49. The pharmaceutical composition of claim 38 wherein said agent is a peptide that acts as a pseudosubstrate for Akt3.

50. The pharmaceutical composition of 49 wherein said peptide acts as a pseudosubstrate for a catalytic domain or a regulatory domain of Akt3.

51. The pharmaceutical composition of 38 wherein said agent is a peptide that acts as a competitive inhibitor for Akt3.

52. The pharmaceutical composition of 51 wherein said peptide acts as a competitive inhibitor for a catalytic domain of Akt3.

53. The pharmaceutical composition of claim 51 wherein said peptide acts as a competitive inhibitor for a pleckstrin homology domain of Akt3.

54. The pharmaceutical composition of claim 51 wherein said peptide acts as a competitive inhibitor for a regulatory domain of Akt3.

55. The pharmaceutical composition of claim 36 wherein said composition further comprises an agent that reduces B-Raf activity.

56. The pharmaceutical composition of claim 55 wherein said agent is selected from the group consisting of:
siRNA molecule, an antisense molecule, an antagonist, a ribozyme, an inhibitor, a peptide, and a small molecule.

57. The pharmaceutical composition of claim 56 wherein said small interfering RNA (siRNA) molecule comprises:
a polynucleotide 5′ GGUCUAGCUACAGAGAAAUCUCGAU 3′ (SEQ ID NO:10), or the complement thereof.

58. The pharmaceutical composition of claim 56 wherein said small interfering RNA (siRNA) molecule comprises:
a polynucleotide 5′ GGACAAAGAAUUGGAUCUGGAUCAU 3′ (SEQ ID NO:11), or the complement thereof.


SEQUENCE LISTING (14 sequences)

SEQ ID NO: 1 - RNA, 19 nt, Homo Sapiens
cuaucuacauuccggaaag

SEQ ID NO: 2 - RNA, 19 nt, Homo Sapiens
gaauuuacagcucagacua

SEQ ID NO: 3 - RNA, 19 nt, Homo Sapiens
cagcucagacuauuacaau

SEQ ID NO: 4 - RNA, 25 nt, Homo Sapiens
cuuggacuaucuacauuccggaaag

SEQ ID NO: 5 - RNA, 25 nt, Homo Sapiens
cuuuccggaauguagauaguccaag

SEQ ID NO: 6 - RNA, 25 nt, Homo Sapiens
gaugaagaauuuacagcucagacua

SEQ ID NO: 7 - RNA, 25 nt, Homo Sapiens
uagucugagcuguaaauucuucauc

SEQ ID NO: 8 - RNA, 25 nt, Homo Sapiens
aauuuacagcucagacuauuacaau

SEQ ID NO: 9 - RNA, 25 nt, Homo Sapiens
auuguaauagucugagcuguaaauu

SEQ ID NO: 10 - RNA, 25 nt, Homo Sapiens
ggucuagcuacagagaaaucucgau

SEQ ID NO: 11 - RNA, 25 nt, Homo Sapiens
ggacaaagaauuggaucuggaucau

SEQ ID NO: 12 - RNA, 25 nt, Homo Sapiens
ggucaaugugcgaaauggaaugagc

SEQ ID NO: 13 - RNA, 25 nt, Homo Sapiens
gaggaacuggacuuccagaagaaca

SEQ ID NO: 14 - DNA, 25 bp, Homo Sapiens
gcacataggagagatgagcttccta